(19) United States
(12) Patent Application Publication
(10) Pub. No.: US 2018/0126014 A1
(43) Pub. Date: May 10, 2018

(54) COMPOSITIONS FOR ENHANCING DELIVERY OF AGENTS ACROSS THE BLOOD BRAIN BARRIER AND METHODS OF USE THEREOF

(71) Applicant: Yale University, New Haven, CT (US)

(72) Inventors: Jiangbing ZHOU, Cheshire, CT (US); Liang HAN, Suzhou (CN); Joseph M. PIEPMEIER, Woodbridge, CT (US)

(21) Appl. No.: 15/566,046

(22) PCT Filed: Apr. 12, 2016
(86) PCT No.: PCT/US2016/027133

§ 371 Date: Oct. 12, 2017

Related U.S. Application Data
(60) Provisional application No. 62/147,942, filed on Apr. 15, 2015.

(51) Int. Cl. A61K 51/12; A61K 47/69; A61K 9/51; A61K 31/7076; A61K 31/506; A61P 35/00; A61P 25/00; A61P 25/28; A61P 25/16; A61P 9/10; A61P 25/08; A61K 47/64

(52) U.S. Cl. CPC A61K 51/1244; A61K 47/6933; A61K 9/5138; A61K 31/7076; A61K 31/506; A61P 35/00; A61K 47/6415; A61P 25/28; A61P 25/16; A61P 9/10; A61P 25/08; A61K 47/645; A61P 25/00

(57) ABSTRACT

Compositions and methods for improved delivery of active agents to the brain are provided. The compositions typically include a nanocarrier, such as a polymeric nanoparticle, liposome, or nanolipagel or are in the form of a conjugate. The nanocarriers or conjugates typically include three components: a targeting moiety; a blood brain barrier blood-brain barrier modulator (BBB modulator), loaded into, attached to the surface of, and/or enclosed within a nanocarrier; and an additional active agent loaded into, attached to the surface of, and/or enclosed within a nanocarrier. The targeting moiety, which is typically conjugated to or otherwise dismodulator played on the surface of the nanocarrier, can be, for example, a moiety that preferentially or specifically targets brain cells or tissue, cancer cells, or a combination thereof.

### CROSS REFERENCE TO RELATED APPLICATIONS

This application claims priority to U.S. Ser. No. 62/147,942 filed Apr. 15, 2015.

### FIELD OF THE INVENTION

The field of the invention generally relates to compositions enhancing delivery of agents across the blood brain barrier, and methods of use thereof.

### BACKGROUND OF THE INVENTION

Brain cancer is a devastating disease. The worldwide incidence of brain cancer, including primary brain cancer and brain metastases, was 256, 000 in 2012 (Ferlay J, et al., Cancer Incidence and Mortality Worldwide: IARC Cancer Base No. 10 [Internet]. Lyon: International Agency for Research on Cancer, 2012 (2013)). Despite surgical and medical advances, the prognosis for most brain cancers remains dismal. The median survival times for glioblastoma—the most common malignant glioma in adults (Scott C B, et al., International Journal of Radiation Oncology, Biology, Physics, 40(1): 51-55 (1998)), diffuse intrinsic pontine glioma—the most common type of brainstem glioma in children (Khatua S, et al., Childs Nery Syst, 27(9):1391-1397 (2011)), and brain metastasis (Jaboin J J, et al., Radiat Oncol, 8 (2013)) are 14 months, 9 months, and 12 months, respectively. Novel therapeutic approaches with improved efficacy for these tumors are urgently needed.

Gene therapy is an effective approach for the treatment of a variety of tumors. However, its application of gene therapy to brain tumors is limited by the lack of efficient delivery platforms that are able to simultaneously overcome the blood-brain barrier (BBB) and cellular barriers. Although local BBB disruption is observed in large brain tumors, these “leaky” blood vessels are located primarily in the tumor center and the capillaries feeding the proliferating tumor edge remain impermeable (Blakeley J., Curr Neurol Neurosci Rep, 8(3): 235-241 (2008)).

The BBB can potentially be bypassed using invasive methods, such as surgical implantation of degradable GLIADEL® wafers, or locoregional administration of Poly(lactic-co-glycolic acid) (PLGA) brain-penetrating nanoparticles (NPs) that were recently developed (Strohbehn G, et al., Journal of Neuro-oncology, 121(3):441-449 (2015); Zhou J, et al., Proc Natl Acad Sci USA, 110(29):11751-11756 (2013)). Unfortunately, the clinical utility of these approaches is hampered by their highly invasive nature. In addition, restricted drug penetration to distant tumor cells that are separate from the tumor bulk limits their therapeutic efficacy (Fung L K, et al., Pharmaceutical Research, 13(5):671-682 (1996); Fung L K, et al., Cancer Research, 58(4):672-684 (1998)). Therefore, next-generation brain cancer gene therapy requires the development of novel technologies that are amenable to systemic delivery and able to target brain tumors.

Nanotechnology represents one of the most promising approaches for intravenous delivery of therapeutic agents to the brain (Deeken J F, et al., Clinical Cancer Research: An Official Journal of the American Association for Cancer Research, 13(6):1663-1674 (2007); Patel T, et al., Advanced Drug Delivery Reviews, 64(7):701-705 (2012); Zhou J, et al., Cancer J, 18(1):89-99 (2012)). The primary benefit of nanotechnology is that NPs can be engineered to take advantage of many mechanisms for brain-targeting delivery including: 1) receptor-mediated transcytosis (Qiao R, et al., ACS Nano, 6(4):3304-3310 (2012)); 2) carrier-mediated transcytosis (Li J, et al., Biomaterials, 34(36):9142-9148 (2013)); 3) adsorptive-mediated transcytosis (Liu L, et al., Biopolymers, 90(5):617-623 (2008)); 4) physical disruption of the BBB (Nance E, et al., Journal of Controlled Release: Official Journal of the Controlled Release Society, 189:123-132 (2014)); and 5) disease microenvironment-targeted delivery (Kievit F M, et al., ACS Nano, 4(8):4587-4594 (2010)).

Nanotechnology also represents the most promising approach for non-viral gene delivery, as synthetic NPs typically have minimal immunogenicity, have potential for surface engineering to allow targeting, and can provide protection of cargo materials that may otherwise be degraded (Zhou J, et al., Cancer J, 18(1):89-99 (2012)). Despite its promise, nanotechnology for systemic gene delivery to the brain is still in its infancy. Existing engineering approaches often fail to enhance systemic delivery of NPs to the brain to a degree sufficient for treatment purposes (Deeken J F, et al., Clinical Cancer Research: An Official Journal of the American Association for Cancer Research, 13(6):1663-1674 (2007); Patel T, et al., Advanced Drug Delivery Reviews, 64(7):701-705 (2012); Zhou J, et al., Cancer J, 18(1):89-99 (2012)). It has been reported that gold NPs can be engineered to cross the BBB and deliver siRNA to brain tumors, providing a survival benefit of several days in mice. However, these inorganic NPs are incapable of carrying large pieces of genetic material and providing protection against degradation (Jensen S A, et al., Science Translational Medicine, 5(209) (2013)). In contrast to inorganic NPs, most existing organic NPs suffer from low delivery efficiency, high toxicity, or both (Zhou J, et al., Cancer J, 18(1):89-99 (2012)). Although several newer generation NPs (Guerrero-Cazares H, et al., ACS Nano, 8(5):5141-5153 (2014); Dahlman J E, et al., Nat Nanotechnol, 9(8):648-655 (2014)), demonstrated excellent efficiency in gene delivery, they do not possess the characteristics optimal for penetrating the BBB.

Therefore, there remains a need for improved ways to delivering active agents to the brain.

It is an object of the invention to provide compositions for increasing delivery of active agent across the blood-brain barrier and into the brain.

It is a further object of the invention to provide method of using the composition for treating diseases and disorders associated with the brain such as brain cancer, neurological and neurodegenerative diseases and disorders, stroke, and injury such as traumatic brain injury.

### SUMMARY OF THE INVENTION

Compositions and methods for improved delivery of active agents to the brain are provided. The compositions typically include a nanocarrier, such as a polymeric nanoparticle, liposome, or nanolipogel. The nanocarriers most typically include three components: a targeting moiety; a blood brain barrier modulator (BBB modulator), loaded into, attached to the surface of, and/or enclosed within a nanocarrier; and an additional active agent loaded into, attached to the surface of, and/or enclosed within a nanocarrier. The targeting moiety, which is typically conjugated to or otherwise displayed on the surface of the nanocarrier, can be, for example, a moiety that preferentially or specifically targets brain cells or tissue, cancer cells, or a combination thereof. In some embodiments, the additional active agent is loaded into or dispersed within a separate nanocarrier from the BBB modulator, or is not loaded into a nanocarrier. For example, in some embodiments, the active agent is free or soluble, or conjugated to the drug. In another embodiment, the BBB modulator, the active agent, and optionally a targeting moiety are conjugated. The conjugate can be administered locally or systemically in a free or soluble form, or packaged into nanocarrier preferably include a targeting moiety.

The compositions are used to improve delivery of the active agent across the blood brain barrier and into the brain. An exemplary strategy is depicted in FIG. 2A. BBB modulator is encapsulated in a nanocarrier and delivered systemically to a subject in need thereof. A fraction of nanocarriers enter the brain through traditional mechanisms. The BBB modulators are then released from the nanocarrier and transiently enhance BBB permeability to more nanocarrier. Through this autocatalytic mechanism, the delivery process creates a positive feedback loop. Consequently, the accumulation efficiency of nanocarrier in the brain increases with time and subsequent administrations. In the most preferred embodiments, the same nanocarriers carrying the BBB modulator also carry an active agent, such as a therapeutic agent or an imaging or contrast agent.

Methods of treating a subject with a disease or disorder using the nanocarrier compositions and autocatalytic strategy are also provided. The methods typically include administering a subject an effective amount brain targeted nanocarriers including a BBB modulator to increase the permeability of the BBB, and an effective of amount of the active agent, preferably also in a nanocarrier, to prevent or alleviate one or more symptoms of the disease or condition. In some embodiments, the dosage of the active agent is lower when administered in combination with the BBB modulator-loaded nanocarrier, but can achieve the same or greater effect than when administered absent the BBB modulator-loaded nanocarrier. In some embodiments, the combination of the BBB modulator-load nanocarrier and active agent can achieve a greater effect than when free BBB modulator and active agent administered in combination at the same dosages. In the most preferred embodiments, the BBB modulator and active agent are both encapsulated or dispersed in a nanocarrier, even more preferably the same nanocarrier.

The methods can be used to treat neurological diseases, including, but not limited to, brain cancer, stroke, injury, epilepsy. In some embodiments, the disclosed nanocarrier compositions including an imaging or contrast agent are employed in a method of imaging the brain of a subject.

### BRIEF DESCRIPTION OF THE DRAWINGS

FIG. 1A is a synthesis diagram for a two-stage process for terpolymerization of high content (40-80%) of lactone with DES and MDEA to synthesize solid terpolymers. The terminal hydroxyl of synthesized unreactive terpolymers reacted with the isocyanate group of PMPI to form functionalized terpolymers. FIG. 1B is a plot showing gene delivery efficiency (RLU/μg protein) of terpolymeric NPs (open diamond) and mHph2-conjugated terpolymeric NPs (open square) in HEK293 cells. FIG. 1C is a line graph showing cytoxicity (cell viability (% of control) as a function of NPs concentration (μg/ml) for plasmid DNA-loaded 111-62% NPs (open round), mHph2-III-62% NPs (solid round) and PEI/DNA polyplexes (open triangle) on HEK293 cells. Cytoxicity was given as the percentage of viable cells remaining after three days of treatment, compared to the control vehicle treated cells. Cell number was determined by the standard MTT assay. All experiments were carried out in triplicate and the standard deviation is denoted using error bars.

FIG. 2A is a schematic of a rationale for autocatalytic delivery of brain tumor-targeted NPs, which is implemented through a combination of targeted delivery by conjugating a tumor-targeting ligand with an autocatalytic mechanism by encapsulating a BBB modulator. FIG. 2B is a plot showing gene delivery efficiency of mHph2-III-62% NPs (open square) and ABTT NPs (open diamond) on GL261 cells. Experiments were carried out in triplicate and the standard deviation is denoted using error bars (data presented as mean±s.d.). FIG. 2C is a curve showing controlled-release of LEXISCAN® from ABTT NPs. Experiments were carried out in triplicate and the standard deviation is denoted using error bars (data presented as mean±s.d.). FIG. 2D is a bar graph showing semi-quantitative fluorescence intensity (FLI) in excised mouse liver, spleen, glioma, kidney, heart, and lung one day after receiving two intravenous administrations of unlabeled CTX-mHph2-III-62% NPs (w/o priming) or ABTT NPs (w/priming) followed by treatment of IR780-loaded ABTT NPs. Mice treated with IR780-labeled mHph2-III-62% NPs were used as controls. All experiments were carried out in triplicate and the standard deviation is denoted using error bars. **** represents p<0.0001 for each comparison.

FIG. 3A is a diagram for synthesis of [¹⁸F]SFB-labeled NPs. FIG. 3B is a line graph showing the dynamic change of radioactivity (F-18 labeled ABTT) with time in tumor (right brain) and corresponding left hemisphere without tumors. The radioactivity within the tumor and the corresponding area of the left hemisphere was quantified based on mean pixel values (PET scan), which was further converted to MBq/mL and standardized to percent of injected dose per gram (% ID/g). Open circles=accumulation of ABTT NPs in tumor, close circles=accumulation of ABTT NPs in the corresponding area of the left hemisphere, open triangles=accumulation of mHph2-III-62% NPs in tumor, closed triangles=accumulation of mHph2-III-62% NPs in the corresponding area of the left hemisphere. FIG. 3C is a curve showing the kinetics of ABTT NP accumulation in brain tumors as measured based on IR780 signal (Brain radiant efficiency (FLI). Experiments were carried out in triplicate and the standard deviations are denoted using error bars (data presented as mean±s.d.). FIG. 3D is a bar graph showing quantitative tissue distribution of ABTT NPs in normal mice.

FIG. 4A is a bar graph showing gene delivery efficiency (RLU/μg protein) of pGL4.13-loaded ABTT NPs (filled bar) and Lipofectamine 2000 (open bar) in GL261 glioma cells. Transfection was performed using the same method as described in FIG. 1. Experiments were carried out in triplicate and the standard deviation is denoted using error bars. Luciferase signal was detected at 6, 12, 24, 48, and 72 h after transfection. Luciferase signal was normalized to the amount of total protein for comparison. **** represents p<0.0001 for each comparison. FIG. 4B is a line graph showing cytotoxicity (cell viability [% of control]) of pB7-1-loaded ABTT NPs (-▴-) on GL261 cells. PEI/DNA polyplexes (-♦-) were used as a control. Toxicity was given as percentage of viable cells remaining after treatment for three days, compared to the control vehicle treated cells. Cell number was determined by the standard MTT assay. Experiments were carried out in triplicate and the standard deviation is denoted using error bars (data presented as mean±s.d.). FIG. 4C is a line graph showing the anti-tumor effects (Tumor volume [mm³]) of pB7-1-loaded ABTT NPs on subcutaneous GL261 tumor (-♦- Blank ABTT NPS, -▴- pB7-1-loaded ABTT NPs). Treatment was initiated when tumors reached a size of ˜50 mm³. Experimental mice received a single intratumoral injection of indicated NPs. Data was given as mean (n=8). FIG. 4D is a Kaplan-Meier survival curve for intracranial GL261 tumor-bearing mice with indicated treatments: pB7-1-loaded ABTT NPs (median survival 38 d); blank ABTT NPs (median survival 28 d); no treatment (median survival 27 d). Each group contained 8 mice. **** represents p<0.0001 for each comparison. FIG. 4E is a Kaplan-Meier survival curves for intracranial U87-MG tumor-bearing mice with indicated treatments: pTRAIL-loaded ABTT NPs (median survival 33 d); saline treatment (median survival 28 d). Each group had 8 mice. Mice treated with pTRAIL-loaded ABTT NPs had significant improvement in median survival compared with saline treatment. p=0.0271 for comparison. Data was given as mean (n=5).

FIG. 5A is a bar graph showing decrease of cerebral blood flow (%) in mice before and during MCAO. FIGS. 5B and 5C are bar graphs showing semi-quantification of NPs (FLI) in liver, spleen, brain, kidney, heart, and lung in models of ischemic (5B) and traumatic (5C) brain injury. For both studies in mice with stroke and TBI, IR780-encapsulated ABTT NPs were administered at 0, 24 and 48 h after surgery. At 24 h after the last injection, fluorescence signal in organs were determined using an IVIS imaging system. Mice treated with 111-62% NPs were used as controls. All experiments were carried out in triplicate. * represents p<0.05.

FIG. 6A is Kaplan-Meier survival curves for mice bearing GL261 gliomas with treatment of paclitaxel-loaded ABTT NPs, blank ABTT NPs, or PBS. FIG. 6B is a Kaplan-Meier survival curve for mice bearing MDA-MB-231Br brain metastases with treatment of paclitaxel-loaded ABTT NPs, free paclitaxel, blank nanoparticles, or PBS. FIG. 6C Kaplan-Meier survival curve of MCAO mice with treatments of PBS, empty NPs, free NEP1-40, or NEP1-40-loaded AIBT NPs. FIG. 6D is a dot plot showing neurological scores of MCAO mice that received indicated treatments at day 3 after surgery.

### DETAILED DESCRIPTION OF THE INVENTION

### I. Definitions

“Small molecule,” as used herein, refers to molecules with a molecular weight of less than about 2000 g/mol, more preferably less than about 1500 g/mol, most preferably less than about 1200 g/mol.

“Nanoparticle”, as used herein, generally refers to a particle having a diameter from about 1 nm up to, but not including, about 1 micron, preferably from 100 nm to about 1 micron. The particles can have any shape. Nanoparticles having a spherical shape can be referred to as “nanospheres”.

“Mean particle size” as used herein, generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

“Monodisperse” and “homogeneous size distribution”, are used interchangeably herein and describe a population of nanoparticles or microparticles where all of the particles are the same or nearly the same size. As used herein, a monodisperse distribution refers to particle distributions in which 90% of the distribution lies within 15% of the median particle size, more preferably within 10% of the median particle size, most preferably within 5% of the median particle size.

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

### II. Compositions

Nanocarrier compositions and formulations thereof are provided. The formulations generally include: a blood brain barrier blood-brain barrier modulator (BBB modulator), loaded into, attached to the surface of, and/or enclosed within a nanocarrier; and an additional active agent loaded into, attached to the surface of, and/or enclosed within a nanocarrier. The BBB modulator and additional active agent can be co-loaded into, attached to the surface of, and/or enclosed within the same nanocarrier, or into separate nanocarriers. The BBB modulator can be conjugated to the active agent, alone or with targeting moiety, with or without a cleavable linker. When the BBB modulator and additional active agent are loaded into, attached to the surface of, and/or enclosed within separate nanocarriers, the nanocarriers can be of the same type (e.g., both PLGA nanoparticles), or different types (e.g., one in PLGA nanoparticles and one in liposomes). The nanocarrier typically includes a targeting moiety, most preferably a moiety that increases targeting to the brain or a cell type within the brain. Accordingly, in the most preferred embodiments, the formulation includes a blood brain barrier blood-brain barrier modulator (BBB modulator), loaded into, attached to the surface of, and/or enclosed within a nanocarrier having a targeting moiety; and an additional active agent loaded into, attached to the surface of, and/or enclosed within the same nanocarrier or a different nanocarrier having a targeting moiety.

A. Nanocarriers

The nanocarriers can be, for example, nanogels, nanolipogels, polymeric particles, lipid particles, hybrid lipid-polymer particles, inorganic particles, liposomes (e.g., nanoliposomes), nanosuspensions, nanoemulsions, multilamellar vesicles, nanofibers, nanorobots, solid lipid nanoparticles (SLN), nanostructured lipid carriers (NLC), or lipid drug conjugates (LDC). In the most preferred embodiments, the particulate nanocarriers are nanoscale compositions, for example, 10 nm up to, but not including, about 1 micron, more preferably up to about 500 nm, as discussed below. However, it will be appreciated that in some embodiments, and for some uses, the particles can be smaller, or larger (e.g., microparticles, etc.). Although the compositions disclosed herein are referred to as nanocarrier compositions throughout, it will be appreciated that in some embodiments and for some uses the particulate compositions can be somewhat larger than nanoparticles. Such compositions can be referred to as microcarrier compositions.

In preferred embodiments for treating diseases and disorders of the brain, it is desirable that the particle be of a size suitable to cross the blood-brain barrier, alone or in combination with a blood-brain barrier modulator. Therefore, the nanocarrier is preferably in the range of about 25 nm to about 500 nm inclusive, more preferably in the range of about 50 nm to about 350 nm inclusive, most preferably between about 70 nm and about 300 nm inclusive.

The nanocarrier can act as drug carriers (e.g, submicroscopic colloidal systems such as nanospheres with a matrix system in which the drug is dispersed) or nanocapsules (e.g., reservoirs in which the drug is confined surrounded by a single polymeric membrane)).

1. Polymeric Particles

a. Polymers

The nanocarrier can be a polymeric particle, for example a micro- or a nanoparticle.

The particles can be biodegradable or non-biodegradable.

Exemplary polymers that can be used to manufacture polymeric particles are discussed above with respect to the polymeric matrix component of particles.

Examples of preferred biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), poly(amine-co-ester), blends and copolymers thereof. In preferred embodiments, the particles are composed of one or more polyesters.

In some embodiments, the one or more polyesters are hydrophobic. For example, particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide, collectively referred to herein as “PLA”, and caprolactone units, such as poly(s-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof.

Additional hydrophobic polymers include, but are not limited to, polyhydroxyalkanoates, polycaprolactones, poly(phosphazenes), polycarbonates, polyamides, polyesteramides, poly(alkylene alkylates), hydrophobic polyethers, polyetheresters, polyacetals, polycyanoacrylates, polyacrylates, polymethylmethacrylates, polysiloxanes, polyketals, polyhydroxyvalerates, polyalkylene oxalates, polyalkylene succinates, and copolymers thereof.

In some embodiments, the polymers are amphiphilic containing a hydrophilic and a hydrophobic polymer described above.

Suitable hydrophilic polymers include, but are not limited to, hydrophilic polypeptides, such as poly-L-glutamic acid, gamma-polyglutamic acid, poly-L-aspartic acid, poly-L-serine, or poly-L-lysine, poly(alkylene glycols) such as polyethylene glycol (PEG), poly(propylene glycol) and copolymers of ethylene glycol and propylene glycol, poly(oxyethylated polyol), poly(olefinic alcohol), polyvinylpyrrolidone), poly(hydroxyalkylmethacrylamide), poly(hydroxyalkylmethacrylate), poly(saccharides), poly (hydroxy acids), poly(vinyl alcohol), as well as copolymers thereof. In some embodiments, the hydrophilic polymer is PEG.

Exemplary amphiphilic polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker.

In some embodiments, the particles are composed of PLGA. PLGA is a safe, FDA approved polymer. PLGA particles are advantageous because they can protect the active agent (i.e., the encapsulant), promote prolonged release, and are amenable to the addition of targeting moieties. For example, the polymer of the particle can have the structure:

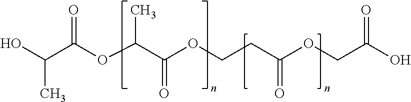

(poly(lactic co-glycolic acid) PLGA+H₂O=variable degradation (days to weeks).

The particles can contain one or more polymer conjugates containing end-to-end linkages between the polymer and a targeting moiety, detectable label, or other active agent. For example, a modified polymer can be a PLGA-PEG-phosphonate. In another example, the particle is modified to include an avidin moiety and a biotinylated targeting moiety, detectable label, or other active agent can be coupled thereto.

Examples of preferred natural polymers include proteins such as albumin, collagen, gelatin and prolamines, for example, zein, and polysaccharides such as alginate, cellulose derivatives and polyhydroxyalkanoates, for example, polyhydroxybutyrate. The in vivo stability of the particles can be adjusted during the production by using polymers such as poly(lactide-co-glycolide) copolymerized with polyethylene glycol (PEG). If PEG is exposed on the external surface, it may increase the time these materials circulate due to the hydrophilicity of PEG.

Examples of preferred non-biodegradable polymers include ethylene vinyl acetate, poly(meth)acrylic acid, polyamides, copolymers and mixtures thereof.

PBCA polymers have been often combined with the nonionic surfactant polysorbate-80 coating and have been proven useful for the delivery of a variety of small polar drugs into the CNS in multiple studies (Grabrucker, et al., “Nanoparticles as Blood-Brain Barrier Permeable CNS Targeted Drug Delivery Systems,” Top Med. Chem., pg. 1-19. DOI: 10.1007/7355_2013_22 (2013). For example, doxorubicin, loperamide, tubocurarine, and dalargin were adsorbed onto PBCA and targeted to the CNS, where they induced a pharmacological effect (Kreuter, “Nanoparticulate systems for brain delivery of drugs,” Adv Drug Deliv Rev, 47:65-81 (2001)).

b. Exemplary Preferred Polymer

In some embodiment the nanocarrier is composed of one or more polymers disclosed in U.S. Published Application No. 2014/0342003. Such polymers are employed in some of the working Examples described in more detail below.

For example, in some embodiments, the polymers have the formula shown below.

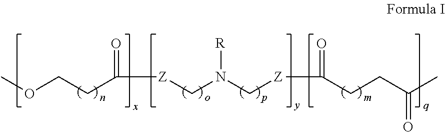

wherein each occurrence of n is an integer from 1-30, each occurrence of m, o, and p is independently an integer from 1-20, and each occurrence of x, y, and q is independently an integer from 1-1000, and Z is O or NR′, wherein R′ is hydrogen, substituted or unsubstituted alkyl, or substituted or unsubstituted aryl.

In some embodiments, Z is O.

In some embodiments, Z is O and n is an integer from 1-16, such as 4, 10, 13, or 14, preferably 10, 13, or 14.

In some embodiments, Z is O, n is an integer from 1-16, such as 4, 10, 13, or 14, and m is an integer from 1-10, such as 4, 5, 6, 7, or 8.

In some embodiments, Z is O, n is an integer from 1-16, such as 4, 10, 13, or 14, preferably 10, 13, or 14, m is an integer from 1-10, such as 4, 5, 6, 7, or 8, and o and p are the same integer from 1-6, such 2, 3, or 4.

In some embodiments, Z is O, n is an integer from 1-16, such as 4, 10, 13, or 14, preferably 10, 13, or 14, m is an integer from 1-10, such as 4, 5, 6, 7, or 8, and R is alkyl, such a methyl, ethyl, n-propyl, isopropyl, n-butyl, or t-butyl, or aryl, such as phenyl.

In certain embodiments, n is 10 (e.g., dodecalactone, DDL), m is 7 (e.g., diethylsebacate, DES), o and p are 2 (e.g., N-methyldiethanolamine, MDEA). In certain embodiments, n, m, o, and p are as defined above, and PEG is incorporated as a monomer.

In certain embodiments, n is 13 (e.g., pentadecalactone, PDL), m is 7 (e.g., diethylsebacate, DES), o and p are 2 (e.g., N-methyldiethanolamine, MDEA). In certain embodiments, n, m, o, and p are as defined above, and PEG is incorporated as a monomer.

In certain embodiments, n is 14 (e.g., hexadecalactone, HDL), m is 7 (e.g., diethylsebacate, DES), o and p are 2 (e.g., N-methyldiethanolamine, MDEA). In certain embodiments, n, m, o, and p are as defined above, and PEG is incorporated as a monomer.

In some embodiments, the polymer is modified with compounds, including but not limited to, p-maleimidophenyl iscocyanate (PMPI) that can be used as a handle for conjugating other compounds or molecules.

The polymers can further include a block of an alkylene oxide, such as polyethylene oxide, polypropylene oxide, and/or polyethylene oxide-co-polypropylene oxide. The structure of a PEG-containing diblock polymer is shown below:

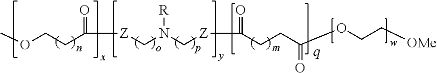

wherein n is an integer from 1-30, m, o, and p are independently an integer from 1-20, x, y, q, and w are independently integers from 1-1000, and Z is O or NR′, wherein R′ is hydrogen, substituted or unsubstituted alkyl, or substituted or unsubstituted aryl. In particular embodiments, the values of x, y, q, and w are such that the weight average molecular weight of the polymer is greater than 20,000 Daltons.

The structure of a PEG-containing triblock copolymer is shown below:

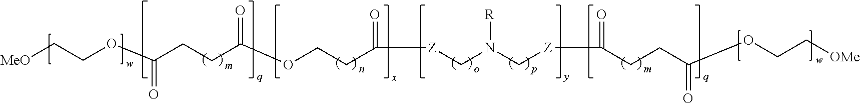

wherein n is an integer from 1-30, m, o, and p are independently an integer from 1-20, x, y, q, and w are independently integers from 1-1000, and Z is O or NR′, wherein R′ is hydrogen, substituted or unsubstituted alkyl, or substituted or unsubstituted aryl. In particular embodiments, the values of x, y, q, and w are such that the weight average molecular weight of the polymer is greater than 20,000 Daltons.

The blocks of polyalkylene oxide can located at the termini of the polymer (i.e., by reacting PEG having one hydroxy group blocked, for example, with a methoxy group), within the polymer backbone (i.e., neither of the hydroxyl groups are blocked), or combinations thereof.

In particular embodiments, the values of x, y, q, and/or w are such that the weight average molecular weight of the polymer is greater than 20,000 Daltons.

The polymer can prepared from one or more lactones, one or more amine-diols (Z=O) or triamines (Z=NR′), and one or more diacids or diesters. In those embodiments where two or more different lactone, diacid or diester, and/or triamine or amine-diol monomers are used, than the values of n, o, p, and/or m can be the same or different.

The monomers shown above can be unsubstituted or can be substituted. “Substituted”, as used herein, means one or more atoms or groups of atoms on the monomer has been replaced with one or more atoms or groups of atoms which are different than the atom or group of atoms being replaced. In some embodiments, the one or more hydrogens on the monomer are replaced with one or more atoms or groups of atoms. Examples of functional groups which can replace hydrogen are listed above in the definition. In some embodiments, one or more functional groups can be added which vary the chemical and/or physical property of the resulting monomer/polymer, such as charge or hydrophilicity/hydrophobicity, etc. Exemplary substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, nitro, heterocyclyl, aralkyl, or an aromatic or heteroaromatic moiety.

The polymer is preferably biocompatible. Readily available lactones of various ring sizes are known to possess low toxicity: for example, polyesters prepared from small lactones, such as poly(caprolactone) and poly(p-dioxanone) are commercially available biomaterials which have been used in clinical applications. Large (e.g., C₁₆-C₂₄) lactones and their polyester derivatives are natural products that have been identified in living organisms, such as bees.

c. Method of Manufacturing Particles

Particles can be prepared using a variety of techniques known in the art. The technique to be used can depend on a variety of factors including the polymer used to form the nanoparticles, the desired size range of the resulting particles, and suitability for the material to be encapsulated.

Suitable techniques include, but are not limited to:

i. Solvent Diffusion/Displacement

In this method, water-soluble or water-miscible organic solvents are used to dissolve the polymer and form emulsion upon mixing with the aqueous phase. The quick diffusion of the organic solvent into water leads to the formation of nanoparticles immediately after the mixing.

ii. Solvent Evaporation

In this method the polymer is dissolved in a volatile organic solvent. The drug (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid nanoparticles. The resulting nanoparticles are washed with water and dried overnight in a lyophilizer. Nanoparticles with different sizes and morphologies can be obtained by this method.

iii. Hot Melt Microencapsulation

In this method, the polymer is first melted and then mixed with the solid particles. The mixture is suspended in a non-miscible solvent (like silicon oil), and, with continuous stirring, heated to 5° C. above the melting point of the polymer. Once the emulsion is stabilized, it is cooled until the polymer particles solidify. The resulting nanoparticles are washed by decantation with petroleum ether to give a free-flowing powder. The external surfaces of spheres prepared with this technique are usually smooth and dense.

iv. Solvent Removal

In this method, the drug is dispersed or dissolved in a solution of the selected polymer in a volatile organic solvent. This mixture is suspended by stirring in an organic oil (such as silicon oil) to form an emulsion. Unlike solvent evaporation, this method can be used to make nanoparticles from polymers with high melting points and different molecular weights. The external morphology of spheres produced with this technique is highly dependent on the type of polymer used.

v. Spray-Drying

In this method, the polymer is dissolved in organic solvent. A known amount of the active drug is suspended (insoluble drugs) or co-dissolved (soluble drugs) in the polymer solution. The solution or the dispersion is then spray-dried.

vi. Phase Inversion

Nanospheres can be formed from polymers using a phase inversion method wherein a polymer is dissolved in a “good” solvent, fine particles of a substance to be incorporated, such as a drug, are mixed or dissolved in the polymer solution, and the mixture is poured into a strong non solvent for the polymer, to spontaneously produce, under favorable conditions, polymeric microspheres, wherein the polymer is either coated with the particles or the particles are dispersed in the polymer. The method can be used to produce nanoparticles in a wide range of sizes, including, for example, about 100 nanometers to about 10 microns. Substances which can be incorporated include, for example, imaging agents such as fluorescent dyes, or biologically active molecules such as proteins or nucleic acids. In the process, the polymer is dissolved in an organic solvent and then contacted with a non-solvent, which causes phase inversion of the dissolved polymer to form small spherical particles, with a narrow size distribution optionally incorporating an antigen or other substance.

vii. Other Methods of Forming Particles

Other methods known in the art that can be used to prepare nanoparticles include, but are not limited to, polyelectrolyte condensation (see Suk et al., Biomaterials, 27, 5143-5150 (2006)); single and double emulsion (probe sonication); nanoparticle molding, and electrostatic self-assembly (e.g., polyethylene imine-DNA or liposome).

In one embodiment, the loaded particles are prepared by combining a solution of the polymer, typically in an organic solvent, with the active agent such as a polynucleotide of interest. The polymer solution is prepared by dissolving or suspending the polymer in a solvent. The solvent should be selected so that it does not adversely effect (e.g., destabilize or degrade) the agent to be encapsulated. Suitable solvents include, but are not limited to DMSO and methylene chloride. The concentration of the polymer in the solvent can be varied as needed. In some embodiments, the concentration is in the range of 25 mg/ml. The polymer solution can also be diluted in a buffer, for example, sodium acetate buffer.

Next, the polymer solution is mixed with the agent to be encapsulated, such as a polynucleotide. The agent can be dissolved in a solvent to form a solution before combining it with the polymer solution. In some embodiments, the agent is dissolved in a physiological buffer before combining it with the polymer solution. The ratio of polymer solution volume to agent solution volume can be 1:1. The combination of polymer and agent are typically incubated for a few minutes to form particles before using the solution for its desired purpose, such as transfection. For example, a polymer/polynucleotide solution can be incubated for 2, 5, 10, or more than 10 minutes before using the solution for transfection. The incubation can be at room temperature.

2. Nanolipogels and Liposomes

The nanocarrier can be a nanolipogel such as those disclosed in WO 2013/155487.

3. Inorganic Particles

The nanocarrier can also be inorganic materials known in the art. See, for example, Barbe, et al., Advanced Materials, 16(21):1959-1966 (2004) and Argyo, et al., Chem. Mater., 26(1):435-451 (2014).

4. Lipid Particles

The particles can contain one or more lipids or amphiphilic compounds. For example, the particles can be liposomes, lipid micelles, solid lipid particles, or lipid-stabilized polymeric particles.

Liposomes, micelles, and other lipid-based nanocarriers useful for preparation of the nanocarrier compositions are known in the art. See, for example, Torchilin, et al., Advanced Drug Delivery Reviews, 58(14):1532-55 (2006).

The lipid particle can be made from one or a mixture of different lipids. Lipid particles are formed from one or more lipids, which can be neutral, anionic, or cationic at physiologic pH. The lipid particle is preferably made from one or more biocompatible lipids. The lipid particles may be formed from a combination of more than one lipid, for example, a charged lipid may be combined with a lipid that is non-ionic or uncharged at physiological pH. Solid lipid particles present an alternative to the colloidal micelles and liposomes. Solid lipid particles are typically submicron in size, i.e. from about 10 nm to about 1 micron, from 10 nm to about 500 nm, or from 10 nm to about 250 nm. Solid lipid particles are formed of lipids that are solids at room temperature. They are derived from oil-in-water emulsions, by replacing the liquid oil by a solid lipid.

The particle can be a lipid micelle. Lipid micelles for drug delivery are known in the art. Lipid micelles can be formed, for instance, as a water-in-oil emulsion with a lipid surfactant. An emulsion is a blend of two immiscible phases wherein a surfactant is added to stabilize the dispersed droplets. In some embodiments the lipid micelle is a microemulsion. A microemulsion is a thermodynamically stable system composed of at least water, oil and a lipid surfactant producing a transparent and thermodynamically stable system whose droplet size is less than 1 micron, from about 10 nm to about 500 nm, or from about 10 nm to about 250 nm. Lipid micelles are generally useful for encapsulating hydrophobic active agents, including hydrophobic therapeutic agents, hydrophobic prophylactic agents, or hydrophobic diagnostic agents.

The particle can be a liposome. Liposomes are small vesicles composed of an aqueous medium surrounded by lipids arranged in spherical bilayers. Liposomes can be classified as small unilamellar vesicles, large unilamellar vesicles, or multi-lamellar vesicles. Multi-lamellar liposomes contain multiple concentric lipid bilayers. Liposomes can be used to encapsulate agents, by trapping hydrophilic agents in the aqueous interior or between bilayers, or by trapping hydrophobic agents within the bilayer. In some embodiments, the nanocarriers are liposomes.

The lipid micelles and liposomes typically have an aqueous center. The aqueous center can contain water or a mixture of water and alcohol. Suitable alcohols include, but are not limited to, methanol, ethanol, propanol, (such as isopropanol), butanol (such as n-butanol, isobutanol, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof. Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-.alpha.-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids.

Suitable cationic lipids include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP); 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N-(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), .beta.-alanyl cholesterol, cetyl trimethyl ammonium bromide (CTAB), diC.sub.14-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N,N,N′,N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)-imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

Suitable solid lipids include, but are not limited to, higher saturated alcohols, higher fatty acids, sphingolipids, synthetic esters, and mono-, di-, and triglycerides of higher saturated fatty acids. Solid lipids can include aliphatic alcohols having 10-40, preferably 12-30 carbon atoms, such as cetostearyl alcohol. Solid lipids can include higher fatty acids of 10-40, preferably 12-30 carbon atoms, such as stearic acid, palmitic acid, decanoic acid, and behenic acid. Solid lipids can include glycerides, including monoglycerides, diglycerides, and triglycerides, of higher saturated fatty acids having 10-40, preferably 12-30 carbon atoms, such as glyceryl monostearate, glycerol behenate, glycerol palmitostearate, glycerol trilaurate, tricaprin, trilaurin, trimyristin, tripalmitin, tristearin, and hydrogenated castor oil. Suitable solid lipids can include cetyl palmitate, beeswax, or cyclodextrin.

Amphiphilic compounds include, but are not limited to, phospholipids, such as 1,2 distearoyl-sn-glycero-3-phosphoethanolamine (DSPE), dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), most preferably between 0.1-30 (weight lipid/w polymer). Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and .beta.-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) may also be used.

5. Conjugates of Therapeutic, Prophylactic or Diagnostic Agent with no carrier

In some embodiments, the targeting moiety and BBB modifying agent are coupled directly to the therapeutic, prophylactic or diagnostic agent to be delivered. These may be coupled via linkers, such as the cleavable linkers described below, so that the agent to be delivered and agent modifying the BBB permeability are released at the same time or sequentially, to achieve greater uptake. In some embodiments, the conjugates are encapsulated within particles that are preferentially taken up at the site of cancer, sepsis, infection or tissue injury, by virtue of size and/or composition, and the conjugates released at these sites for greater penetration into the brain.

B. Targeting Moieties

Typically, one or more targeting moieties (also referred to herein as targeting molecules, and targeting signals) can be loaded into, attached to the surface of, and/or enclosed within the nanocarrier. Exemplary target molecules include proteins, peptides, nucleic acids, lipids, saccharides, or polysaccharides that bind to one or more targets associated with an organ, tissue, cell, or extracellular matrix, or specific type of tumor or infected cell. Preferably, the targeting moiety is displayed on and preferably conjugated to the exterior surface of the nanocarrier. Preferably, the targeting moiety increases or enhances targeting of the nanocarrier to the brain. In some embodiments, the targeting moiety increases or enhances targeting of the nanocarrier to the BBB, and/or to brain cells, preferably diseased or abnormal brain cells. In some embodiments, the targeting moiety increases or enhances targeting of the nanocarrier to cells in the brain that are not brain cells. For example, the targeting moiety can increase targeting of the nanocarrier to cancer cells that were not originally derived from a brain cell (e.g., brain metastases). Various techniques can be used to engineer the surface of nanocarriers, such as covalent linkage of molecules (ligands) to nanosystems (polymers or lipids) (Tosi, et al., SfN Neurosci San Diego (USA), 1:84 (2010)).

The degree of specificity with which the nanocarriers are targeted can be modulated through the selection of a targeting molecule with the appropriate affinity and specificity. For example, antibodies are very specific. These can be polyclonal, monoclonal, fragments, recombinant, or single chain, many of which are commercially available or readily obtained using standard techniques. T-cell specific molecules and antigens which are bound by antigen presenting cells as well as tumor targeting molecules can be bound to the surface of the nanocarrier. The targeting molecules may be conjugated to the terminus of one or more PEG chains present on the surface of the particle.

In some embodiments, the targeting moiety is an antibody or antigen binding fragment thereof that specifically recognizes a tumor marker that is present exclusively or in elevated amounts on a malignant cell (e.g., a tumor antigen). Fragments are preferred since antibodies are very large, and can have limited diffusion through tissue. Suitable targeting molecules that can be used to direct the nanocarrier to cells and tissues of interest, as well as methods of conjugating target molecules to nanoparticles, are known in the art. See, for example, Ruoslahti, et al. Nat. Rev. Cancer, 2:83-90 (2002).

Targeting molecules can also include neuropilins and endothelial targeting molecules, integrins, selectins, adhesion molecules, cytokines, and chemokines.

In some embodiments, the targeting moiety is an antibody or an antibody binding domain in combination with an antibody binding domain. The antibody can be polyclonal, monoclonal, linear, humanized, chimeric or a fragment thereof. The antibody can be antibody fragment such as Fab, Fab′, F(ab′), Fv diabody, linear antibody, or single chain antibody. Antibody binding domains are known in the art and include, for example, proteins as Protein A and Protein G from Staphylococcus aureus. Other domains known to bind antibodies are known in the art and can be substituted.

Targeting molecules can be covalently bound to nanocarriers using a variety of methods known in the art. In preferred embodiments the targeting moiety is attached to the nanocarrier by PEGylation or a biotin-avidin bridge. The density of the targeting moiety can be important, depended on the affinity of a given moiety with cells or tissues of interest and stereospecific blockade. The density of moiety is preferable in the range of 20 to 1,000,000 per nanocarrier, more preferable 50 to 10,000 per nanocarrier.

1. Brain Targeting

In some embodiments, the targeting signal is directed to cells of the nervous system, including the brain and peripheral nervous system, or for the blood-brain barrier itself. Cells in the brain include several types and states and possess unique cell surface molecules specific for the type. Furthermore, cell types and states can be further characterized and grouped by the presentation of common cell surface molecules.

The targeting signal can be directed to specific neurotransmitter receptors expressed on the surface of cells of the nervous system. The distribution of neurotransmitter receptors is well known in the art and one so skilled can direct the compositions described by using neurotransmitter receptor specific antibodies as targeting signals. Furthermore, given the tropism of neurotransmitters for their receptors, in one embodiment the targeting signal consists of a neurotransmitter or ligand capable of specifically binding to a neurotransmitter receptor.

The targeting signal can be specific to cells of the nervous system which may include astrocytes, microglia, neurons, oligodendrites and Schwann cells. These cells can be further divided by their function, location, shape, neurotransmitter class and pathological state. Cells of the nervous system can also be identified by their state of differentiation, for example stem cells Exemplary markers specific for these cell types and states are well known in the art and include, but are not limited to CD133 and Neurosphere.

Specific preferred brain targeting moieties are provided below in the working Examples, and include, but are not limited to, the peptide mHph2 and the peptide chlorotoxin (CTX).

The mode of transport of particles across the BBB is believed to be mediated by passive diffusion and/or receptor-mediated endocytosis, fluid phase endocytosis or phagocytosis, carrier-mediated transport or by absorptive-mediated transcytosis (Grabrucker, et al., “Nanoparticles as Blood-Brain Barrier Permeable CNS Targeted Drug Delivery Systems,” Top Med. Chem., pg. 1-19, DOI: 10.1007/7355_2013_22 (2013)). Passive diffusion can be enhanced by increasing the composition's plasma concentration, resulting in a larger gradient at the BBB and thus an increase in the amount of composition entering the CNS. One strategy for introducing nanocarriers into the brain is receptor-mediated endocytosis. This strategy relies on the interaction of the particle surface ligand with a specific receptor in the BBB. Examples of suitable ligands include transferrin, transferrin receptor binding antibody, lactoferrin, melanotransferrin, folic acid, and a-mannose for NPs undergoing receptor-mediated transcytosis. (Grabrucker, et al., “Nanoparticles as Blood-Brain Barrier Permeable CNS Targeted Drug Delivery Systems,” Top Med. Chem., pg. 1-19, DOI: 10.1007/7355_2013_22 (2013)). It is believe that nanocarriers engineered with such targeting moieties interact with the targeted receptor, create endocytotic vesicles, transcytosis across the BBB endothelial cells, and are subsequently exocytosed. Besides playing a role in nanocarrier uptake, surface engineering can be used to target different cell compartments. Because the vascular density in the brain is very high, once nanocarriers have crossed the BBB, they will spread rapidly throughout the brain.

Therefore, in some embodiments, the targeting moiety targets, preferably by binding to, a BBB marker. Markers and even specific targeting moieties thereto, are known in the art and include, but are not limited to, transfer receptor (which can be targeted by, for example, OX26 antibody, and 8D3 antibody), insulin receptor (which can be target by, for example, 83-14 antibody or insulin), EGF receptor (which can be target by, for example, centuximad and fragments (e.g., Fab′) thereof), low-density lipoprotein receptor (which can be targeted by, for example, apolipoproteins such as ApoA, ApoE, etc.), thiamine receptor (which can be targeted with, for example, thiamine), transferrin receptor (which can be targeted with, for example, transferrin), folate receptor (which can be targeted with, for example, transferrin), glycoside receptor (which can be targeted with, for example, glycosides), lactoferrin receptor (which can be targeted with, for example, lactoferrin), insulin-like growth factor receptors (IGF1R & IGF2R) (which can be targeted with, for example, insulin like growth factor 1 & 2 (IGF-1 & IGF-2), and mannose-6-phosphate), leptin receptor (LEPR) (which can be targeted with, for example, leptin), Fc like growth factor receptor (FCGRT) (which can be target with, for example, IgG), scavenger receptor type B1 (SCARB1) (which can be targeted with, for example, (modified lipoproteins, like acetylated low density lipoprotein (LDL)), and others targets and targeting moieties discussed in Alam, et al., European Journal of Pharmaceutical Sciences, 40:385-403 (2010), and Wong, et al., Adv Drug Deliv Rev., 64(7):686-700 (2012)).

In other embodiment, the markers are related to, or specific for, the condition being treated. For example, in some embodiments, the target moiety targets a marker of cancer (discussed in more detail below), stroke (e.g., MMP2, thrombin), epilepsy (e.g., MMP2), injury, or a neurological or neurodegenerative disease or disorder.

2. Tumor-Specific and Tumor-Associated Antigens

In some embodiment, the targeting moiety is an antigen that is expressed by tumor cells. The antigen expressed by the tumor may be specific to the tumor, or may be expressed at a higher level on the tumor cells as compared to non-tumor cells. Antigenic markers such as serologically defined markers known as tumor associated antigens, which are either uniquely expressed by cancer cells or are present at markedly higher levels (e.g., elevated in a statistically significant manner) in subjects having a malignant condition relative to appropriate controls, are known.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melonoma associated antigens, etc.) (see, e.g., U.S. Pat. No. 6,699,475; Jager, et al., Int. J. Cancer, 106:817-20 (2003); Kennedy, et al., Int. Rev. Immunol., 22:141-72 (2003); Scanlan, et al. Cancer Immun., 4:1 (2004)).

Genes that encode cellular tumor associated antigens include cellular oncogenes and proto-oncogenes that are aberrantly expressed. In general, cellular oncogenes encode products that are directly relevant to the transformation of the cell, so these antigens are particularly preferred targets for immunotherapy. An example is the tumorigenic neu gene that encodes a cell surface molecule involved in oncogenic transformation. Other examples include the ras, kit, and trk genes. The products of proto-oncogenes (the normal genes which are mutated to form oncogenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

In ovarian and other carcinomas, for example, tumor associated antigens are detectable in samples of readily obtained biological fluids such as serum or mucosal secretions. One such marker is CA125, a carcinoma associated antigen that is also shed into the bloodstream, where it is detectable in serum (e.g., Bast, et al., N Eng. J. Med., 309:883 (1983); Lloyd, et al., Int. J. Canc., 71:842 (1997). CA125 levels in serum and other biological fluids have been measured along with levels of other markers, for example, carcinoembryonic antigen (CEA), squamous cell carcinoma antigen (SCC), tissue polypeptide specific antigen (TPS), sialyl TN mucin (STN), and placental alkaline phosphatase (PLAP), in efforts to provide diagnostic and/or prognostic profiles of ovarian and other carcinomas (e.g., Sarandakou, et al., Acta Oncol., 36:755 (1997); Sarandakou, et al., Eur. J. Gynaecol. Oncol., 19:73 (1998); Meier, et al., Anticancer Res., 17(4B):2945 (1997); Kudoh, et al., Gynecol. Obstet. Invest., 47:52 (1999)), all of which can metastasize to the brain. Elevated serum CA125 may also accompany neuroblastoma (e.g., Hirokawa, et al., Surg. Today, 28:349 (1998), while elevated CEA and SCC, among others, may accompany colorectal cancer (Gebauer, et al., Anticancer Res., 17(4B):2939 (1997)).

The tumor associated antigen mesothelin, defined by reactivity with monoclonal antibody K-1, is present on a majority of squamous cell carcinomas including epithelial ovarian, cervical, and esophageal tumors, and on mesotheliomas (Chang, et al., Cancer Res., 52:181 (1992); Chang, et al., Int. J. Cancer, 50:373 (1992); Chang, et al., Int. J. Cancer, 51:548 (1992); Chang, et al., Proc. Natl. Acad. Sci. USA, 93:136 (1996); Chowdhury, et al., Proc. Natl. Acad. Sci. USA, 95:669 (1998)). Using MAb K-1, mesothelin is detectable only as a cell-associated tumor marker and has not been found in soluble form in serum from ovarian cancer patients, or in medium conditioned by OVCAR-3 cells (Chang, et al., Int. J. Cancer, 50:373 (1992)). Structurally related human mesothelin polypeptides, however, also include tumor-associated antigen polypeptides such as the distinct mesothelin related antigen (MRA) polypeptide, which is detectable as a naturally occurring soluble antigen in biological fluids from patients having malignancies (see WO 00/50900).

A tumor antigen may include a cell surface molecule. Tumor antigens of known structure and having a known or described function, include the following cell surface receptors: HER1 (GenBank Accession NO: U48722), HER2 (Yoshino, et al., J. Immunol., 152:2393 (1994); Disis, et al., Canc. Res., 54:16 (1994); GenBank Acc. Nos. X03363 and M17730), HER3 (GenBank Acc. Nos. U29339 and M34309), HER4 (Plowman, et al., Nature, 366:473 (1993); GenBank Acc. Nos. L07868 and T64105), epidermal growth factor receptor (EGFR) (GenBank Acc. Nos. U48722, and K03193), vascular endothelial cell growth factor (GenBank NO: M32977), vascular endothelial cell growth factor receptor (GenBank Acc. Nos. AF022375, 1680143, U48801 and X62568), insulin-like growth factor-I (GenBank Acc. Nos. X00173, X56774, X56773, X06043, European Patent No. GB 2241703), insulin-like growth factor-II (GenBank Acc. Nos. X03562, X00910, M17863 and M17862), transferrin receptor (Trowbridge and Omary, Proc. Nat. Acad. USA, 78:3039 (1981); GenBank Acc. Nos. X01060 and M11507), estrogen receptor (GenBank Acc. Nos. M38651, X03635, X99101, U47678 and M12674), progesterone receptor (GenBank Acc. Nos. X51730, X69068 and M15716), follicle stimulating hormone receptor (FSH-R) (GenBank Acc. Nos. Z34260 and M65085), retinoic acid receptor (GenBank Acc. Nos. L12060, M60909, X77664, X57280, X07282 and X06538), MUC-1 (Barnes, et al., Proc. Nat. Acad. Sci. USA, 86:7159 (1989); GenBank Acc. Nos. M65132 and M64928) NY-ESO-1 (GenBank Acc. Nos. AJ003149 and U87459), NA 17-A (PCT Publication NO: WO 96/40039), Melan-A/MART-1 (Kawakami, et al., Proc. Nat. Acad. Sci. USA, 91:3515 (1994); GenBank Acc. Nos. U06654 and U06452), tyrosinase (Topalian, et al., Proc. Nat. Acad. Sci. USA, 91:9461 (1994); GenBank Acc. NO: M26729; Weber, et al., J Clin. Invest, 102:1258 (1998)), Gp-100 (Kawakami, et al., Proc. Nat. Acad. Sci. USA, 91:3515 (1994); GenBank Acc. NO: S73003, Adema, et al., J. Biol. Chem., 269:20126 (1994)), MAGE (van den Bruggen, et al., Science, 254:1643 (1991)); GenBank Acc. Nos. U93163, AF064589, U66083, D32077, D32076, D32075, U10694, U10693, U10691, U10690, U10689, U10688, U10687, U10686, U10685, L18877, U10340, U10339, L18920; U03735 and M77481), BAGE (GenBank Acc. NO: U19180; U.S. Pat. Nos. 5,683,886 and 5,571,711), GAGE (GenBank Acc. Nos. AF055475, AF055474, AF055473, U19147, U19146, U19145, U19144, U19143 and U19142), any of the CTA class of receptors including in particular HOM-MEL-40 antigen encoded by the SSX2 gene (GenBank Acc. Nos. X86175; U90842, U90841 and X86174), carcinoembryonic antigen (CEA, Gold and Freedman, J. Exp. Med., 121:439 (1985); GenBank Acc. Nos. M59710, M59255 and M29540), and PyLT (GenBank Acc. Nos. J02289 and J02038); p97 (melanotransferrin) (Brown, et al., J Immunol., 127:539-46 (1981); Rose, et al., Proc. Natl. Acad. Sci. USA, 83:1261-61 (1986)).

Additional tumor associated antigens include prostate surface antigen (PSA) (U.S. Pat. Nos. 6,677,157; 6,673,545); β-human chorionic gonadotropin β-HCG) (McManus, et al., Cancer Res., 36:3476-81 (1976); Yoshimura, et al., Cancer, 73:2745-52 (1994); Yamaguchi, et al., Br. J. Cancer, 60:382-84 (1989): Alfthan, et al., Cancer Res., 52:4628-33 (1992)); glycosyltransferase β-1,4-N-acetylgalactosaminyltransferases (GalNAc) (Hoon, et al., Int. J. Cancer, 43:857-62 (1989); Ando, et al., Int. J. Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession NO: X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Tumor antigens of interest include antigens regarded in the art as “cancer/testis” (CT) antigens that are immunogenic in subjects having a malignant condition (Scanlan, et al., Cancer Immun., 4:1 (2004)). CT antigens include at least 19 different families of antigens that contain one or more members and that are capable of inducing an immune response, including, but not limited to, MAGEA (CT1); BAGE (CT2); MAGEB (CT3); GAGE (CT4); SSX (CT5); NY-ESO-1 (CT6); MAGEC (CT7); SYCP1 (C8); SPANXB1 (CT11.2); NA88 (CT18); CTAGE (CT21); SPA17 (CT22); OY-TES-1 (CT23); CAGE (CT26); HOM-TES-85 (CT28); HCA661 (CT30); NY-SAR-35 (CT38); FATE (CT43); and TPTE (CT44).

Additional tumor antigens that can be targeted, including a tumor-associated or tumor-specific antigen, include, but are not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3, 4, 5, 6, 7, GnTV, Herv-K-mel, Lage-1, Mage-A1, 2, 3, 4, 6, 10, 12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, a-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS. Other tumor-associated and tumor-specific antigens are known to those of skill in the art and are suitable for targeting by the disclosed fusion proteins.

3. Antigens Associated with Tumor Neovasculature

Tumor-associated neovasculature provides a readily accessible route through which therapeutics can access the tumor. In one embodiment the viral proteins contain a domain that specifically binds to an antigen that is expressed by neovasculature associated with a tumor.

The antigen may be specific to tumor neovasculature or may be expressed at a higher level in tumor neovasculature when compared to normal vasculature. Exemplary antigens that are over-expressed by tumor-associated neovasculature as compared to normal vasculature include, but are not limited to, VEGF/KDR, Tie2, vascular cell adhesion molecule (VCAM), endoglin and α₅β₃ integrin/vitronectin. Other antigens that are over-expressed by tumor-associated neovasculature as compared to normal vasculature are known to those of skill in the art and are suitable for targeting by the disclosed fusion proteins.

4. Chemokines/Chemokine Receptors

In another embodiment, the nanocarriers contain a targeting moiety that specifically binds to a chemokine, cytokine, or a receptor thereof. Chemokines are soluble, small molecular weight (8-14 kDa) proteins that bind to their cognate G-protein coupled receptors (GPCRs) to elicit a cellular response, usually directional migration or chemotaxis. Tumor cells secrete and respond to chemokines, which facilitate growth that is achieved by increased endothelial cell recruitment and angiogenesis, subversion of immunological surveillance and maneuvering of the tumoral leukocyte profile to skew it such that the chemokine release enables the tumor growth and metastasis to distant sites. Thus, chemokines are vital for tumor progression.

Based on the positioning of the conserved two N-terminal cysteine residues of the chemokines, they are classified into four groups: CXC, CC, CX3C and C chemokines. The CXC chemokines can be further classified into ELR+ and ELR− chemokines based on the presence or absence of the motif ‘glu-leu-arg (ELR motif)’ preceding the CXC sequence. The CXC chemokines bind to and activate their cognate chemokine receptors on neutrophils, lymphocytes, endothelial and epithelial cells. The CC chemokines act on several subsets of dendritic cells, lymphocytes, macrophages, eosinophils, natural killer cells but do not stimulate neutrophils as they lack CC chemokine receptors except murine neutrophils. There are approximately 50 chemokines and only 20 chemokine receptors, thus there is considerable redundancy in this system of ligand/receptor interaction.

Chemokines elaborated from the tumor and the stromal cells bind to the chemokine receptors present on the tumor and the stromal cells. The autocrine loop of the tumor cells and the paracrine stimulatory loop between the tumor and the stromal cells facilitate the progression of the tumor. Notably, CXCR2, CXCR4, CCR2 and CCR7 play major roles in tumorigenesis and metastasis. CXCR2 plays a vital role in angiogenesis and CCR2 plays a role in the recruitment of macrophages into the tumor microenvironment. CCR7 is involved in metastasis of the tumor cells into the sentinel lymph nodes as the lymph nodes have the ligand for CCR7, CCL21. CXCR4 is mainly involved in the metastatic spread of a wide variety of tumors.

In some embodiments the targeting moiety targets (e.g., binds to) inflammation or a maker associated therewith, for example an inflammatory cytokine, chemokine, or receptor thereof. Inflammatory chemokines are known in the art and include, but are not limited to IL-1I3, TNF-α, TGF-beta, IFN-γ, IL-17, IL-6, IL-23, IL-22, IL-21, and matrix metalloproteinases (MMPs).

C. Blood-Brain Barrier Modulators

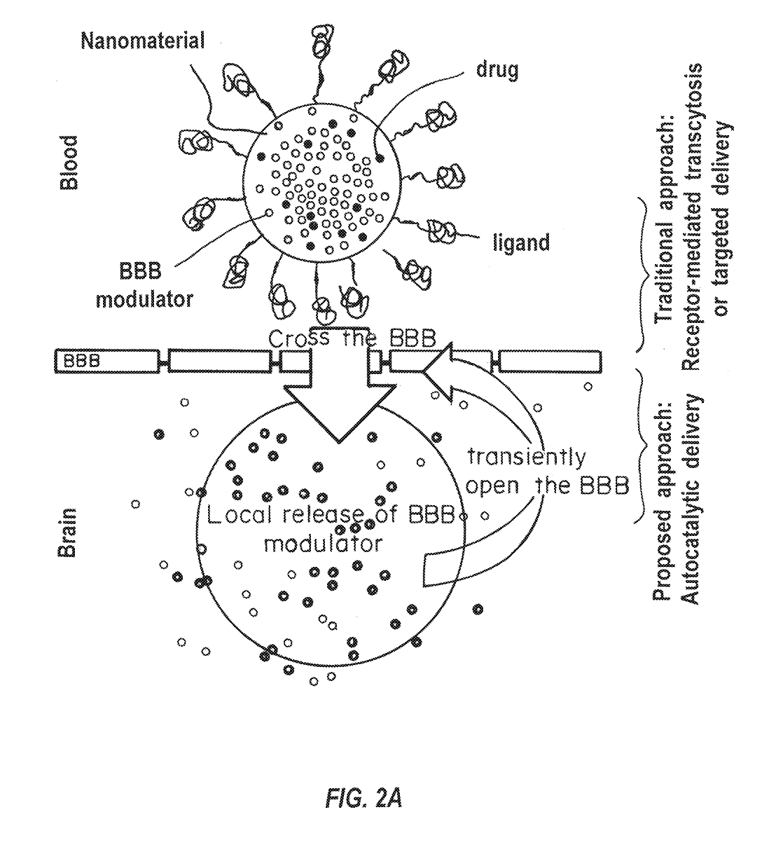

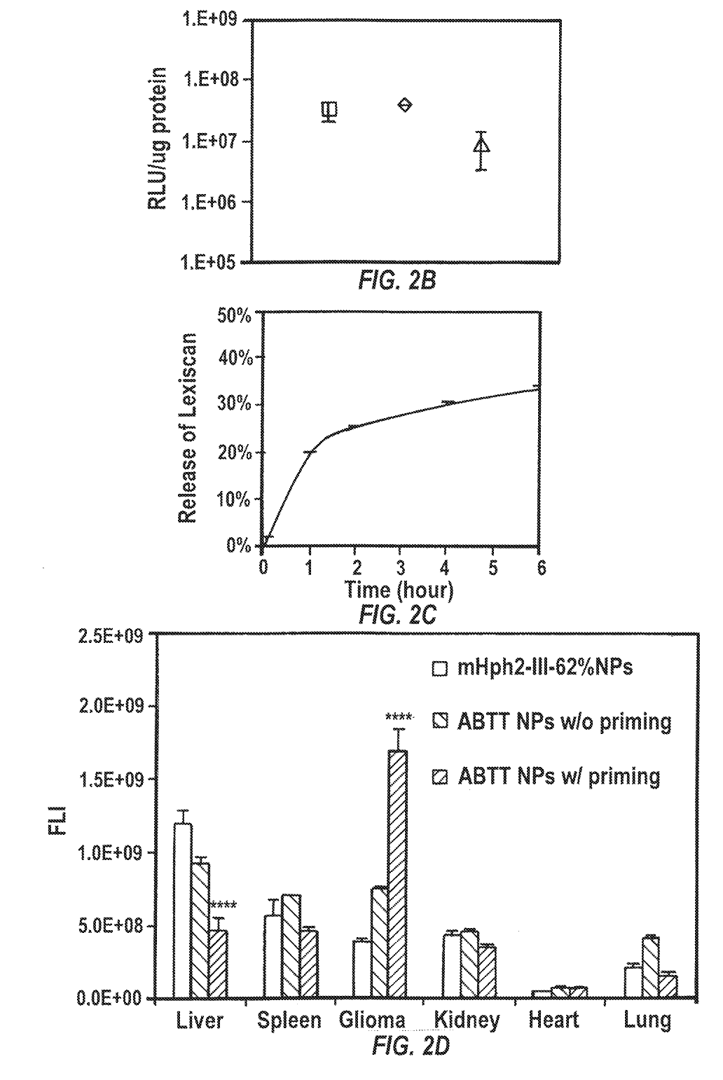

The nanocarrier compositions typically include one or more blood-brain barrier modulators. The compositions are designed to overcome limited delivery of materials across the blood-brain barrier and typically rely on an autocatalytic feedback mechanism. An exemplary embodiment is depicted in FIG. 2A in which BBB modulators are encapsulated in nanoparticles (NPs) and delivered locally, or preferably systemically to a subject in need thereof. A fraction of NPs enter the brain tumor microenvironment through traditional mechanisms. The BBB modulators are then released from the NPs and transiently enhance BBB permeability to more NPs. Through this autocatalytic mechanism, the delivery process creates a positive feedback loop. Consequently, the accumulation efficiency of NPs in the tumor increases with time and subsequent administrations.

Therefore, the nanocarriers loaded with BBB modulators are typically administered to a subject in need thereof in an amount effective to increase permeability of the BBB to the nanocarriers. For example, the BBB modulator can be loaded into or onto the nanocarrier and released therefrom after systemic administration to subject in need thereof in amount effective to increase BBB permeability in less than about 48 hours after systemic administration, preferably in less than about 24 hours after systemic administration, preferably in less than about 12 hours after systemic administration, preferably about 6 hours after systemic administration. In particular embodiments, the BBB modulator is loaded into or onto the nanocarrier and released therefrom after systemic administration to subject in need thereof in amount effective to increase BBB permeability within about 4 to about 10 hours after systemic administration, preferably within about 6 to about 10 hours after systemic administration. The BBB permeability can be increased in an effective amount to increase the crossing of nanocarriers or even free or soluble active agents across the BBB and into the brain.

Typically, the BBB is loaded into or onto the nanocarrier in at a concentration of about 0.5% to about 5.0% by weight of the nanocarrier, though higher and lower concentration can also be effective.

The blood-brain barrier (BBB) is comprised of brain endothelial cells (BECs), which form the lumen of the brain microvasculature (Abbott et al., Neurobiol Dis., 37:13-25 (2010)). The barrier function is achieved through tight junctions between endothelial cells that regulate the extravasation of molecules and cells into and out of the central nervous system (CNS). Modulation of adenosine receptor (AR) signaling at BECs is known to modulate BBB permeability and to facilitate the entry of molecules and cells into the CNS (Carman, et al., The Journal of Neuroscience, 31(37):13272-13280 (2011)). Accordingly, in some embodiment, the BBB modulator is an AR agonist.

An exemplary AR agonist NECA (CAS No.: 35920-39-9; 1-(6-Amino-9H-purin-9-yl)-1-deoxy-N-ethyl-β-D-ribofuranuronamide). NECA is a broad spectrum AR agonist that activates all ARs (A1, A2A, A2B, A3), and is known in increase BBB permeability to macromolecules (Carman, et al., The Journal of Neuroscience, 31(37):13272-13280 (2011)).

Regadenoson (INN, code named CVT-3146, 2-{4-[(methylamino)carbonyl]-1H-pyrazol-1-yl}adenosine) is an A2A adenosine receptor agonist that is a coronary vasodilator. Regadenoson is an A2A adenosine receptor agonist that is a coronary vasodilator. Regadenoson is chemically described as adenosine, 2-[4-[(methylamino)carbonyl]-1H-pyrazol-1-yl]-, monohydrate. The molecular formula for regadenoson is C₁₅H₁₈N₈O₅.H₂O and its molecular weight is 408.37. Lexiscan is a sterile, nonpyrogenic solution for intravenous injection. The solution is clear and colorless. Each 1 mL in the 5 mL pre-filled syringe contains 0.084 mg of regadenoson monohydrate, corresponding to 0.08 mg regadenoson on an anhydrous basis, 10.9 mg dibasic sodium phosphate dihydrate or 8.7 mg dibasic sodium phosphate anhydrous, 5.4 mg monobasic sodium phosphate monohydrate, 150 mg propylene glycol, 1 mg edetate disodium dihydrate, and Water for Injection, with pH between 6.3 and 7.7. It is marketed by Astellas Pharma under the traclename LEXISCAN® for increasing blood flow through the arteries of the heart during cardiac nuclear stress testing. Regadenoson is a low affinity agonist (Ki≈1.3 μM) for the A2A adenosine receptor, with at least 10-fold lower affinity for the A1 adenosine receptor (Ki>16.5 μM), and weak, if any, affinity for the A2B and A3 adenosine receptors. Activation of the A2A adenosine receptor by regadenoson produces coronary vasodilation and increases coronary blood flow (CBF). Regadenoson is also a BBB permeability agent (Carman, et al., The Journal of Neuroscience, 31(37):13272-13280 (2011)).

In another embodiment, the BBB modulator is minoxidil sulfate (MS). MS is an adenosine 5′-triphosphate-sensitive potassium channel (KATP channel) activator, which is known to selectively increase the permeability of the blood-tumor barrier (BTB) (Gu, et al., Neuropharmacology, 75:407-15 (2013)).

In another embodiment, the BBB modulator is borneol. Borneol (CAS No.: 507-70-0; endo-1,7,7-Trimethyl-bicyclo[2.2.1]heptan-2-01) is a bicyclic organic compound and a terpene. Borneol is widely used in traditional Chinese medicine to enhance delivery of central nervous system (CNS) drugs to the brain because it can increase permeability of the BBB (Yu, et al., J Ethnopharmacol., 150(3):1096-108 (2013)).

The BBB modulator can be compounds which stimulate TNF-alpha production, including, but not limited to, ST013006 (N-[[5-(3-bromophenyl)furan-2-yl]methylideneamino]pyridine-3-carboxamide) (Schepetkin, et al, Mol Pharmacol., 74(2):392-402 (2008)). TNF-alpha production can locally produce inflammation, resulting in partial BBB disruption (Qiao, et al, Oncotarget, 2(1-2):59-68 (2011).

In some embodiment, the nanocarrier, conjugant or other element of the composition includes a protein transduction domain or a cell penetrating peptide. The PTD can be a polypeptide, polynucleotide, carbohydrate, or organic or inorganic compounds that facilitate traversing a lipid bilayer, micelle, cell membrane, organelle membrane, or vesicle membrane. A PTD attached to another molecule facilitates the molecule traversing membranes, for example going from extracellular space to intracellular space, or cytosol to within an organelle. Exemplary PTDs include, but are not limited to, HIV TAT; 11 Arginine residues, or positively charged polypeptides or polynucleotides having 8-15 residues, preferably 9-11 residues.

D. Therapeutic, Prophylactic or Diagnostic Agents

The nanocarrier compositions include one or more therapeutic, prophylactic, or diagnostic active agents loaded into, attached to the surface of, and/or enclosed within the nanocarrier or conjugated to the BBB modulator and/or targeting agent. In some embodiments, two, three, four, or more active agents are loaded into, attached to the surface of, and/or enclosed within the nanocarrier. The two or more agents can be loaded into, attached to the surface of, and/or enclosed within the same particle, or different particles. In some embodiments, the formulation includes two or more different types of particles having the same or different active agent(s) associated therewith. In some embodiments, additional active agents are co-administered to the subject but are not loaded into, attached to the surface of, and/or enclosed within the disclosed nanocarrier(s), and can be, for example, free or soluble, or in a different carrier or dosage form. For example, such active agents can be free or soluble active agent(s), or active agent(s) in a different carrier or dosage form but are nonetheless part of the same pharmaceutical composition as the nanocarrier composition.

The active agents can be small molecule active agents or biomacromolecules, such as proteins, polypeptides, or nucleic acids. In some embodiments, the nucleic acid is an expression vector encoding a protein or a functional nucleic acid. Vectors can be suitable for integration into a cell genome or expressed extra-chomasomally. In other embodiments, the nucleic acid is a functional nucleic acid. Suitable small molecule active agents include organic and organometallic compounds. The small molecule active agents can be hydrophilic, hydrophobic, or amphiphilic compounds. The active agent can be a therapeutic, nutritional, diagnostic, or prophylactic agent.

Exemplary active agents include, but are not limited to, chemotherapeutic agents, neurological agents, tumor antigens, CD4+ T-cell epitopes, cytokines, imaging agents, radionuclides, small molecule signal transduction inhibitors, photothermal antennas, immunologic danger signaling molecules, other immunotherapeutics, enzymes, antibiotics, antivirals, anti-parasites, growth factors, growth inhibitors, hormones, hormone antagonists, antibodies and bioactive fragments thereof (including humanized, single chain, and chimeric antibodies), antigen and vaccine formulations (including adjuvants), peptide drugs, anti-inflammatories, immunomodulators (including ligands that bind to Toll-Like Receptors (including, but not limited to, CpG oligonucleotides) to activate the innate immune system, molecules that mobilize and optimize the adaptive immune system, molecules that activate or up-regulate the action of cytotoxic T lymphocytes, natural killer cells and helper T-cells, and molecules that deactivate or down-regulate suppressor or regulatory T-cells), agents that promote uptake of the nanocarrier into cells (including dendritic cells and other antigen-presenting cells), nutraceuticals such as vitamins, oligonucleotide drugs (including DNA, RNAs, antisense, aptamers, small interfering RNAs, ribozymes, external guide sequences for ribonuclease P, and triplex forming agents) and other gene modifying agents such as ribozymes, CRISPR/Cas, zinc finger nuclease, and transcription activator-like effector nucleases (TALEN).

Exemplary diagnostic agents include paramagnetic molecules, fluorescent compounds, magnetic molecules, and radionuclides, x-ray imaging agents, and contrast agents.

1. Chemotherapeutic Agents

In certain embodiments, the nanocarrier includes one or more anti-cancer agents. Representative anti-cancer agents include, but are not limited to, alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), topoisomerase inhibitors (including camptothecins such as camptothecin, irinotecan, and topotecan as well as derivatives of epipodophyllotoxins such as amsacrine, etoposide, etoposide phosphate, and teniposide), antibodies to vascular endothelial growth factor (VEGF) such as bevacizumab (AVASTIN®), other anti-VEGF compounds; thalidomide (THALOMID®) and derivatives thereof such as lenalidomide (REVLIMID®); endostatin; angiostatin; receptor tyrosine kinase (RTK) inhibitors such as sunitinib (SUTENT®); tyrosine kinase inhibitors such as sorafenib (Nexavar®), erlotinib (Tarceva®), pazopanib, axitinib, and lapatinib; transforming growth factor-α or transforming growth factor-β inhibitors, and antibodies to the epidermal growth factor receptor such as panitumumab (VECTIBIX®) and cetuximab (ERBITUX®).

In preferred embodiments, particularly those for treating cancer, one or more of the active agents can be a chemotherapeutic agent that has immune signaling properties.

2. Neurological Agents

In some embodiment that active agent is a conventional treatment for neurodegeneration, or for increasing or enhancing neuroprotection. Exemplary neuroprotective agents are known in the art in include, for example, glutamate antagonists, antioxidants, and NMDA receptor stimulants. Other neuroprotective agents and treatments include caspase inhibitors, trophic factors, anti-protein aggregation agents, therapeutic hypothermia, and erythropoietin. Amantadine and anticholinergics are used for treating motor symptoms, clozapine for treating psychosis, cholinesterase inhibitors for treating dementia. Treatment strategies can also include administration of modafinil.

For subjects with Huntington's disease, dopamine blocker is used to help reduce abnormal behaviors and movements, and drugs such as amantadine and tetrabenazine are used to control movement, etc. Drugs that help to reduce chorea include neuroleptics and benzodiazepines. Compounds such as amantadine or remacemide have shown preliminary positive results. Hypokinesia and rigidity, especially in juvenile cases, can be treated with antiparkinsonian drugs, and myoclonic hyperkinesia can be treated with valproic acid. Psychiatric symptoms can be treated with medications similar to those used in the general population. Selective serotonin reuptake inhibitors and mirtazapine have been recommended for depression, while atypical antipsychotic drugs are recommended for psychosis and behavioral problems.

Treatments for Parkinson's disease, include, but are not limited to, levodopa (usually combined with a dopa decarboxylase inhibitor or COMT inhibitor), dopamine agonists, and MAO-B inhibitors.

The only compound yielding borderline significance with respect to survival time in subjects with ALS is riluzole (RILUTEK®) (2-amino-6-(trifluoromethoxy) benzothiazole), an antiexcitotoxin. Other medications, most used off-label, and interventions can reduce symptoms due to ALS. Some treatments improve quality of life and a few appear to extend life. Common ALS-related therapies are reviewed in Gordon, Aging and Disease, 4(5):295-310 (2013), which is specifically incorporated by reference herein in its entirety. Exemplary ALS treatments and interventions are also discussed in Gordon, Aging and Disease, 4(5):295-310 (2013), listed in a table provided therein.

A number of other agents have been tested in one or more clinical trials with efficacies ranging from non-efficacious to promising. Exemplary agents are reviewed in Carlesi, et al., Archives Italiennes de Biologie, 149:151-167 (2011) and include, for example, agents that reduces excitotoxicity such as talampanel (8-methyl-7H-1,3-dioxolo(2,3)benzodiazepine), a cephalosporin such as ceftriaxone, or memantine; agents that reduce oxidative stress such as coenzyme Q10, manganoporphyrins, KNS-760704 [(6R)-4,5,6,7-tetrahydro-N6-propyl-2,6-benzothiazole-diamine dihydrochloride, RPPX], and edaravone (3-methyl-1-phenyl-2-pyrazolin-5-one, MCI-186); agents that reduces apoptosis such as histone deacetylase (HDAC) inhibitors including valproic acid, TCH346 (Dibenzo(b,f)oxepin-10-ylmethyl-methylprop-2-ynylamine), minocycline, or tauroursodeoxycholic Acid (TUDCA); agents that reduce neuroinflammation such as thalidomide and celastol; neurotropic agents such as insulin-like growth factor 1 (IGF-1) and vascular endothelial growth factor (VEGF); heat shock protein inducers such as arimoclomol; or an autophagy inducer such as rapamycin or lithium.

Exemplary neurological drugs include, but are not limited to, ABSTRAL® (fentanyl), AGGRENOX® (aspirin/extended-release dipyridamole), AMERCE® (naratriptan), AMPYRA® (dalfampridine), AMRIX® (cyclobenzaprine hydrochloride extended release), ANEXSIA®, APOKYN® (apomorphine hydrochloride), APTIOM® (eslicarbazepine acetate), ARICEPT® (donepezil hydrochloride), asprin, AVINZA® (morphine sulfate), AVONEX® (Interferon Beta 1-A), AXERT® (almotriptan malate), AXONA® (caprylidene), BANZEL® (rufinamide), BELSOMRA® (suvorexant), BOTOX® (onabotulinumtoxinA), BROMDAY® (bromfenac), BUTRANS® (buprenorphine), CAMBIA® (diclofenac potassium), CARBAGLU® (carglumic acid), CARBATROL® (Carbamazepine), CENESTIN® (synthetic conjugated estrogens, A), CIALIS® (tadalafil), KLONOPIN® (clonazepam), COMTAN® (Entacapone), COPAXONE® (glatiramer acetate), CUVPOSA® (glycopyrrolate), CYLERT®, DEPAKOTE® (divalproex sodium), DEPAKOTE® (divalproex sodium), DEPAKOTE ER® (divalproex sodium), DUOPA® (carbidopa and levodopa), DUREZOL® (difluprednate), DYLOJECT® (diclofenac sodium), EDLUAR® (zolpidem tartrate), ELIQUIS® (apixaban), EMBEDA® (morphine sulfate and naltrexone hydrochloride), EXALGO® (hydromorphone hydrochloride), EXELON® (rivastigmine tartrate), EXELON® (rivastigmine tartrate), EXPAREL® (bupivacaine liposome injectable suspension), EXTAVIA® (Interferon beta-1 b), FETZIMA® (levomilnacipran), FOCALIN® (dexmethylphenidate HCl), FROVA® (frovatriptan succinate), FYCOMPA® (perampanel), GALZIN® (zinc acetate), GRALISE® (gabapentin), HETLIOZ® (tasimelteon), HORIZANT® (gabapentin enacarbil), HORIZANT® (gabapentin enacarbil), IMITREX® (sumatriptan), IMITREX® (sumatriptan), INTERMEZZO® (zolpidem tartrate sublingual tablet), INTUNIV® (guanfacine extended-release), INVEGA® (paliperidone), NUMBY® (iontocaine), KADIAN® (Morphine Sulfate), KAPVAY® (clonidine hydrochloride), LEVETIRACTAM® (keppra), LAMICTAL® (lamotrigine), LAZANDA® (fentanyl citrate). LEMTRADA® (alemtuzumab), LEVITRA® (vardenafil), LUNESTA® (eszopiclone), LUPRON DEPOT® (leuprolide acetate), LUSEDRA® (fospropofol disodium), LYRICA® (pregabalin), MAXALT® (rizatriptan benzoate), MERREM I.V.® (meropenem), METADATE CD® (methylphenidate HCl), MIGRANAL® (dihydroergotamine), MIRAPEX® (pramipexole), MOVANTIK® (naloxegol), MYOBLOC® (rimabotulinumtoxinB), REVIA® (naltrexone hydrochloride), NAMENDA® (memantine HCl), NAMZARIC® (memantine hydrochloride extended-release+donepezil hydrochloride), NEUPRO® (Rotigotine Transdermal System), NEUPRO® (rotigotine), NEURONTIN® (gabapentin), NORCO® (Hydrocodone Bitartrate/Acetaminophen 10 mg/325 mg), NORTHERA® (droxidopa), NOVANTRONE® (mitoxantrone hydrochloride), NUCYNTA® (tapentadol), NUEDEXTA® (dextromethorphan hydrobromide and quinidine sulfate), NUVIGIL® (armodafinil), NYMALIZE® (nimodipine), ONFI® (clobazam), ONSOLIS® (fentanyl buccal), OXECTA® (oxycodone HCl), OXTELLAR XR® (oxcarbazepine extended release), OXYCONTIN® (oxycodone), PERCODAN® (oxycodone/aspirin), PERCOCET® (oxycodone with acetaminophen), PLEGRIDY® (peginterferon beta-la), POSICOR® (mibefradil), POTIGA® (ezogabine), QUADRAMET® (samarium lexidronam), QUDEXY XR® (topiramate), QUILLIVANT XR® (methylphenidate hydrochloride), QUTENZA® (capsaicin), REBIF® (interferon beta-1a), REDUX® (dexfenfluramine hydrochloride), RELPAX® (eletriptan hydrobromide), REMINYL® (galantamine hydrobromide), REQUIP® (ropinirole hydrochloride), RILUTEK® (riluzole), ROZEREM® (ramelteon), RYTARY® (carbidopa and levodopa), SABRIL® (vigabatrin), ZELAPAR® (selegiline), SILENOR® (doxepin), SONATA® (zaleplon), SPRIX® (ketorolac tromethamine), STAVZOR® (valproic acid delayed release), STRATTERA® (atomoxetine HCl), SUBSYS® (fentanyl sublingual spray), TARGINIQ ER® (oxycodone hydrochloride+naloxone hydrochloride), TASMAR® (tolcapone), TEGRETOL® (carbamazepine), TIVORBEX® (indomethacin), TOPAMAX® (topiramate), TRILEPTAL® (oxcarbazepine), TROKENDI XR® (topiramate), TYSABRI® (natalizumab), ULTRACET® (acetaminophen and tramadol HCl), ULTRAJECT VERSED® (midazolam HCl), VIIBRYD® (vilazodone hydrochloride), VIMPAT® (lacosamide), VISIPAQUE® (iodixanol), VIVITROL® (naltrexone), VPRIV® (velaglucerase alfa), VYVANSE® (Lisdexamfetamine Dimesylate), XARTEMIS XR® (oxycodone hydrochloride and acetaminophen), XENAZINE® (tetrabenazine), XIFAXAN® (rifaximin), XYREM® (sodium oxybate), ZANAFLEX® (tizanidine hydrochloride), ZINGO® (lidocaine hydrochloride monohydrate), ZIPSOR® (diclofenac potassium), ZOHYDRO ER® (hydrocodone bitartrate), ZOMIG® (zolmitriptan), ZONEGRAN® (zonisamide), ZUBSOLV® (buprenorphine and naloxone).

3. Immune Modulators

The active agent can be an immunomodulator such as an immune response stimulating agent or an agent that blocks immunosuppression. In particularly preferred embodiments, the active agents target tumor checkpoint blockade or costimulatory molecules.

The immune system is composed of cellular (T-cell driven) and humoral (B-cell driven) elements. It is generally accepted that for cancer, triggering of a powerful cell-mediated immune response is more effective than activation of humoral immunity. Cell-based immunity depends upon the interaction and co-operation of a number of different immune cell types, including antigen-presenting cells (APC; of which dendritic cells are an important component), cytotoxic T cells, natural killer cells and T-helper cells. Therefore, the active agent can be an agent that increases a cell (T-cell driven) immune response, a humoral (B-cell driven) immune response, or a combination thereof. For example, in some embodiments, the agent enhances a T cell response, increases T cell activity, increases T cell proliferation, reduces a T cell inhibitory signal, enhances production of cytokines, stimulates T cell differentiation or effector function, promotes survival of T cells or any combination thereof.

Exemplary immunomodulatory agents include cytokines, xanthines, interleukins, interferons, oligodeoxynucleotides, glucans, growth factors (e.g., TNF, CSF, GM-CSF and G-CSF), hormones such as estrogens (diethylstilbestrol, estradiol), androgens (testosterone, HALOTESTIN® (fluoxymesterone)), progestins (MEGACE® (megestrol acetate), PROVERA® (medroxyprogesterone acetate)), and corticosteroids (prednisone, dexamethasone, hydrocortisone).

In some embodiments the agent is an inflammatory molecule such as a cytokine, metelloprotease or other molecule including, but not limited to, IL-1β, TNF-α, TGF-beta, IFN-γ, IL-17, IL-6, IL-23, IL-22, IL-21, and MMPs.

a. Cytokines

In a preferred embodiment, at least one of the active agents is a proinflammatory cytokine. Cytokines typically act as hormonal regulators or signaling molecules at nano- to- picomolar concentrations and help in cell signaling. The cytokine can be a protein, peptide, or glycoprotein. Exemplary cytokines include, but are not limited to, interleukins (e.g., IL-1, IL-2, IL-4, IL-5, IL-6, IL-7, IL-12, IL-15, etc.), interferons (e.g., interferon-γ), macrophage colony stimulating factor, granulocyte colony stimulating factor, tumor necrosis factor, Leukocyte Inhibitory Factor (LIF), chemokines, SDF-1α, and the CXC family of cytokines.

b. Chemokines

In another embodiment, at least one of the active agents is a proinflammatory chemokine. Chemokines are a family of small cytokines. Their name is derived from their ability to induce directed chemotaxis in nearby responsive cells. Therefore, they are chemotactic cytokines. Proteins are classified as chemokines according to shared structural characteristics such as small size (they are all approximately 8-10 kDa in size), and the presence of four cysteine residues in conserved locations that are key to forming their 3-dimensional shape. Chemokines have been classified into four main subfamilies: CXC, CC, CX3C and XC. Chemokines induce cell signaling by binding to G protein-linked transmembrane receptors (i.e., chemokine receptors).

4. Agents that Block Immune Suppression

At least one of the active agents can be an agent that blocks, inhibits or reduces immune suppression or that that blocks, inhibits or reduces the bioactivity of a factor that contributes to immune suppression. It has become increasingly clear that tumor-associated immune suppression not only contributes greatly to tumor progression but is also one of the major factors limiting the activity of cancer immunotherapy. Antigen-specific T-cell tolerance is one of the major mechanisms of tumor escape, and the antigen-specific nature of tumor non-responsiveness indicates that tumor-bearing hosts are not capable of maintaining tumor-specific immune responses while still responding to other immune stimuli (Willimsky, et al., Immunol. Rev., 220:102-12 (2007), Wang, et al. Semin Cancer Biol., 16:73-9 (2006), Frey, et al., Immunol. Rev., 222:192-205 (2008), Nagaraj, et al., Clinical Cancer Research, 16(6):1812-23 (2010)).

5. Polynucleotides

The nanoparticles can include a nucleic acid cargo. The polynucleotide can encode one or more proteins, can encode or be functional nucleic acids, or combinations thereof. The polynucleotide can be monocistronic or polycistronic. In some embodiments, the polynucleotide is multigenic. In some embodiments, the polynucleotide is transfected into the cell and remains extrachromosomal. In some embodiments, the polynucleotide is introduced into a host cell and is integrated into the host cell's genome. As discussed in more detail below, the compositions can be used in methods of gene therapy. Methods of gene therapy can include the introduction into the cell of a polynucleotide that alters the genotype of the cell. Introduction of the polynucleotide can correct, replace, or otherwise alter the endogenous gene via genetic recombination. Methods can include introduction of an entire replacement copy of a defective gene, a heterologous gene, or a small nucleic acid molecule such as an oligonucleotide. For example, a corrective gene can be introduced into a non-specific location within the host's genome.

In some embodiments, the polynucleotide is incorporated into or part of a vector. Methods to construct expression vectors containing genetic sequences and appropriate transcriptional and translational control elements are well known in the art. These methods include in vitro recombinant DNA techniques, synthetic techniques, and in vivo genetic recombination. Expression vectors generally contain regulatory sequences and necessary elements for the translation and/or transcription of the inserted coding sequence, which can be, for example, the polynucleotide of interest. The coding sequence can be operably linked to a promoter and/or enhancer to help control the expression of the desired gene product. Promoters used in biotechnology are of different types according to the intended type of control of gene expression. They can be generally divided into constitutive promoters, tissue-specific or development-stage-specific promoters, inducible promoters, and synthetic promoters.

For example, in some embodiments, the polynucleotide of interest is operably linked to a promoter or other regulatory elements known in the art. Thus, the polynucleotide can be a vector such as an expression vector. The engineering of polynucleotides for expression in a prokaryotic or eukaryotic system may be performed by techniques generally known to those of skill in recombinant expression. An expression vector typically comprises one of the compositions under the control of one or more promoters. To bring a coding sequence “under the control of” a promoter, one positions the 5′ end of the translational initiation site of the reading frame generally between about 1 and 50 nucleotides “downstream” of (i.e., 3′ of) the chosen promoter. The “upstream” promoter stimulates transcription of the inserted DNA and promotes expression of the encoded recombinant protein. This is the meaning of “recombinant expression” in the context used here.

Many standard techniques are available to construct expression vectors containing the appropriate nucleic acids and transcriptional/translational control sequences in order to achieve protein or peptide expression in a variety of host-expression systems. Cell types available for expression include, but are not limited to, bacteria, such as E. coli and B. subtilis transformed with recombinant phage DNA, plasmid DNA or cosmid DNA expression vectors. It will be appreciated that any of these vectors may be packaged and delivered using the disclosed polymers.

Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter). Further, it is also possible, and may be desirable, to utilize promoter or control sequences normally associated with the desired gene sequence, provided such control sequences are compatible with the host cell systems.

A number of viral based expression systems may be utilized, for example, commonly used promoters are derived from polyoma, Adenovirus 2, cytomegalovirus and Simian Virus 40 (SV40). The early and late promoters of SV40 virus are useful because both are obtained easily from the virus as a fragment which also contains the SV40 viral origin of replication. Smaller or larger SV40 fragments may also be used, provided there is included the approximately 250 bp sequence extending from the HindIII site toward the BglI site located in the viral origin of replication.

In cases where an adenovirus is used as an expression vector, the coding sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing proteins in infected hosts.

Specific initiation signals may also be required for efficient translation of the disclosed compositions. These signals include the ATG initiation codon and adjacent sequences. Exogenous translational control signals, including the ATG initiation codon, may additionally need to be provided. One of ordinary skill in the art would readily be capable of determining this need and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements or transcription terminators.

In eukaryotic expression, one will also typically desire to incorporate into the transcriptional unit an appropriate polyadenylation site if one was not contained within the original cloned segment. Typically, the poly A addition site is placed about 30 to 2000 nucleotides “downstream” of the termination site of the protein at a position prior to transcription termination.

For long-term, high-yield production of recombinant proteins, stable expression is preferred. For example, cell lines that stably express constructs encoding proteins may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci, which in turn can be cloned and expanded into cell lines.

a. Polypeptide of Interest

The polynucleotide can encode one or more polypeptides of interest. The polypeptide can be any polypeptide. For example, the polypeptide encoded by the polynucleotide can be a polypeptide that provides a therapeutic or prophylactic effect to an organism or that can be used to diagnose a disease or disorder in an organism. For example, for treatment of cancer, autoimmune disorders, parasitic, viral, bacterial, fungal or other infections, the polynucleotide(s) to be expressed may encode a polypeptide that functions as a ligand or receptor for cells of the immune system, or can function to stimulate or inhibit the immune system of an organism.

In some embodiments, the polynucleotide supplements or replaces a polynucleotide that is defective in the organism.

In some embodiments, the polynucleotide includes a selectable marker, for example, a selectable marker that is effective in a eukaryotic cell, such as a drug resistance selection marker. This selectable marker gene can encode a factor necessary for the survival or growth of transformed host cells grown in a selective culture medium. Typical selection genes encode proteins that confer resistance to antibiotics or other toxins, e.g., ampicillin, neomycin, methotrexate, kanamycin, gentamycin, Zeocin, or tetracycline, complement auxotrophic deficiencies, or supply critical nutrients withheld from the media.

In some embodiments, the polynucleotide includes a reporter gene. Reporter genes are typically genes that are not present or expressed in the host cell. The reporter gene typically encodes a protein which provides for some phenotypic change or enzymatic property. Examples of such genes are provided in Weising et al. Ann. Rev. Genetics; 22, 421 (1988). Preferred reporter genes include glucuronidase (GUS) gene and GFP genes.

b. Functional Nucleic Acids

The polynucleotide can be, or can encode a functional nucleic acid. Functional nucleic acids are nucleic acid molecules that have a specific function, such as binding a target molecule or catalyzing a specific reaction. Functional nucleic acid molecules can be divided into the following non-limiting categories: antisense molecules, siRNA, miRNA, aptamers, ribozymes, triplex forming molecules, RNAi, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the polypeptide itself. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. There are numerous methods for optimization of antisense efficiency by finding the most accessible regions of the target molecule. Exemplary methods include in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹².

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with K_{d}'s from the target molecule of less than 10⁻¹² M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule. It is preferred that the aptamer have a K_{d} with the target molecule at least 10, 100, 1000, 10,000, or 100,000 fold lower than the K_{d} with a background binding molecule. It is preferred when doing the comparison for a molecule such as a polypeptide, that the background molecule be a different polypeptide.

Ribozymes are nucleic acid molecules that are capable of catalyzing a chemical reaction, either intramolecularly or intermolecularly. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes. There are also a number of ribozymes that are not found in natural systems, but which have been engineered to catalyze specific reactions de novo. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence.

Triplex forming functional nucleic acid molecules are molecules that can interact with either double-stranded or single-stranded nucleic acid. When triplex molecules interact with a target region, a structure called a triplex is formed in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹².

External guide sequences (EGSs) are molecules that bind a target nucleic acid molecule forming a complex, which is recognized by RNase P, which then cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukarotic cells. Representative examples of how to make and use EGS molecules to facilitate cleavage of a variety of different target molecules are known in the art.

Gene expression can also be effectively silenced in a highly specific manner through RNA interference (RNAi). This silencing was originally observed with the addition of double stranded RNA (dsRNA) (Fire, et al. (1998) Nature, 391:806-11; Napoli, et al. (1990) Plant Cell 2:279-89; Hannon, (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, et al. (2001) Genes Dev., 15:188-200; Bernstein, et al. (2001) Nature, 409:363-6; Hammond, et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, et al. (2001) Nature, 411:494 498) (Ui-Tei, et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit.

The production of siRNA from a vector is more commonly done through the transcription of a short hairpin RNAse (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors.

Other inhibitory nucleic acids include miRNA and piRNA.

c. Composition of the Polynucleotides

The polynucleotide can be DNA or RNA nucleotides which typically include a heterocyclic base (nucleic acid base), a sugar moiety attached to the heterocyclic base, and a phosphate moiety which esterifies a hydroxyl function of the sugar moiety. The principal naturally-occurring nucleotides comprise uracil, thymine, cytosine, adenine and guanine as the heterocyclic bases, and ribose or deoxyribose sugar linked by phosphodiester bonds.

The polynucleotide can be composed of nucleotide analogs that have been chemically modified to improve stability, half-life, or specificity or affinity for a target sequence, relative to a DNA or RNA counterpart. The chemical modifications include chemical modification of nucleobases, sugar moieties, nucleotide linkages, or combinations thereof. As used herein ‘modified nucleotide” or “chemically modified nucleotide” defines a nucleotide that has a chemical modification of one or more of the heterocyclic base, sugar moiety or phosphate moiety constituents. In some embodiments, the charge of the modified nucleotide is reduced compared to DNA or RNA oligonucleotides of the same nucleobase sequence. For example, the oligonucleotide can have low negative charge, no charge, or positive charge. Modifications should not prevent, and preferably enhance, the ability of the oligonucleotides to enter a cell and carry out a function such inhibition of gene expression as discussed above.

Typically, nucleoside analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). Preferred analogs are those having a substantially uncharged, phosphorus containing backbone.

As discussed in more detail below, in one preferred embodiment, the oligonucleotide is a morpholino oligonucleotide.

i. Heterocyclic Bases

The principal naturally-occurring nucleotides include uracil, thymine, cytosine, adenine and guanine as the heterocyclic bases. The oligonucleotides can include chemical modifications to their nucleobase constituents. Chemical modifications of heterocyclic bases or heterocyclic base analogs may be effective to increase the binding affinity or stability in binding a target sequence. Chemically-modified heterocyclic bases include, but are not limited to, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-.beta.-D-ribofuranosyl)pyridine (2-aminopyridine), and various pyrrolo- and pyrazolopyrimidine derivatives.

ii. Sugar Modifications

Polynucleotides can also contain nucleotides with modified sugar moieties or sugar moiety analogs. Sugar moiety modifications include, but are not limited to, 2′-O-aminoetoxy, 2′-O-amonioethyl (2′-OAE), 2′-O-methoxy, 2′-O-methyl, 2-guanidoethyl (2′-OGE), 2′-O,4′-C-methylene (LNA), 2′-O-(methoxyethyl) (2′-OME) and 2′-O-(N-(methyl)acetamido) (2′-OMA). 2′-O-aminoethyl sugar moiety substitutions are especially preferred because they are protonated at neutral pH and thus suppress the charge repulsion between the TFO and the target duplex. This modification stabilizes the C3′-endo conformation of the ribose or dexyribose and also forms a bridge with the i-1 phosphate in the purine strand of the duplex.

The polynucleotide can be a morpholino oligonucleotide. Morpholino oligonucleotides are typically composed of two more morpholino monomers containing purine or pyrimidine base-pairing moieties effective to bind, by base-specific hydrogen bonding, to a base in a polynucleotide, which are linked together by phosphorus-containing linkages, one to three atoms long, joining the morpholino nitrogen of one monomer to the 5′ exocyclic carbon of an adjacent monomer. The purine or pyrimidine base-pairing moiety is typically adenine, cytosine, guanine, uracil or thymine. The synthesis, structures, and binding characteristics of morpholino oligomers are detailed in U.S. Pat. Nos. 5,698,685, 5,217,866, 5,142,047, 5,034,506, 5,166,315, 5,521,063, and 5,506,337.

Important properties of the morpholino-based subunits typically include: the ability to be linked in a oligomeric form by stable, uncharged backbone linkages; the ability to support a nucleotide base (e.g. adenine, cytosine, guanine, thymidine, uracil or inosine) such that the polymer formed can hybridize with a complementary-base target nucleic acid, including target RNA, with high Tₘ, even with oligomers as short as 10-14 bases; the ability of the oligomer to be actively transported into mammalian cells; and the ability of an oligomer:RNA heteroduplex to resist RNAse degradation. In some embodiments, oligonucleotides employ morpholino-based subunits bearing base-pairing moieties, joined by uncharged linkages.

iii. Internucleotide Linkages

Internucleotide bond refers to a chemical linkage between two nucleoside moieties. Modifications to the phosphate backbone of DNA or RNA oligonucleotides may increase the binding affinity or stability polynucleotides, or reduce the susceptibility of polynucleotides to nuclease digestion. Cationic modifications, including, but not limited to, diethyl-ethylenediamide (DEED) or dimethyl-aminopropylamine (DMAP) may be especially useful due to decrease electrostatic repulsion between the oligonucleotide and a target. Modifications of the phosphate backbone may also include the substitution of a sulfur atom for one of the non-bridging oxygens in the phosphodiester linkage. This substitution creates a phosphorothioate internucleoside linkage in place of the phosphodiester linkage. Oligonucleotides containing phosphorothioate intemucleoside linkages have been shown to be more stable in vivo.

Examples of modified nucleotides with reduced charge include modified internucleotide linkages such as phosphate analogs having achiral and uncharged intersubunit linkages (e.g., Sterchak, et al., Organic Chem., 52:4202, (1987)), and uncharged morpholino-based polymers having achiral intersubunit linkages (see, e.g., U.S. Pat. No. 5,034,506), as discussed above. Some internucleotide linkage analogs include morpholidate, acetal, and polyamide-linked heterocycles.

In another embodiment, the oligonucleotides are composed of locked nucleic acids. Locked nucleic acids (LNA) are modified RNA nucleotides (see, for example, Braasch, et al., Chem. Biol., 8(1):1-7 (2001)). LNAs form hybrids with DNA which are more stable than DNA/DNA hybrids, a property similar to that of peptide nucleic acid (PNA)/DNA hybrids. Therefore, LNA can be used just as PNA molecules would be. LNA binding efficiency can be increased in some embodiments by adding positive charges to it. Commercial nucleic acid synthesizers and standard phosphoramidite chemistry are used to make LNAs.

In some embodiments, the oligonucleotides are composed of peptide nucleic acids. Peptide nucleic acids (PNAs) are synthetic DNA mimics in which the phosphate backbone of the oligonucleotide is replaced in its entirety by repeating N-(2-aminoethyl)-glycine units and phosphodiester bonds are typically replaced by peptide bonds. The various heterocyclic bases are linked to the backbone by methylene carbonyl bonds. PNAs maintain spacing of heterocyclic bases that is similar to conventional DNA oligonucleotides, but are achiral and neutrally charged molecules. Peptide nucleic acids are comprised of peptide nucleic acid monomers.

Other backbone modifications include peptide and amino acid variations and modifications. Thus, the backbone constituents of oligonucleotides such as PNA may be peptide linkages, or alternatively, they may be non-peptide peptide linkages. Examples include acetyl caps, amino spacers such as 8-amino-3,6-dioxaoctanoic acid (referred to herein as O-linkers), amino acids such as lysine are particularly useful if positive charges are desired in the PNA. Methods for the chemical assembly of PNAs are well known. See, for example, U.S. Pat. Nos. 5,539,082, 5,527,675, 5,623,049, 5,714,331, 5,736,336, 5,773,571, and 5,786,571.

Polynucleotides optionally include one or more terminal residues or modifications at either or both termini to increase stability, and/or affinity of the oligonucleotide for its target. Commonly used positively charged moieties include the amino acids lysine and arginine, although other positively charged moieties may also be useful. For example, lysine and arginine residues can be added to a bis-PNA linker or can be added to the carboxy or the N-terminus of a PNA strand. Polynucleotides may further be modified to be end capped to prevent degradation using a 3′ propylamine group. Procedures for 3′ or 5′ capping oligonucleotides are well known in the art.

E. Conjugates

As noted above, the targeting agent, the BBB modulator and/or the active agent can be linked directly or indirectly via the nanocarrier.

As used herein, the term “linker” refers to a carbon chain that can contain heteroatoms (e.g., nitrogen, oxygen, sulfur, etc.) and which may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 atoms long. Linkers may be substituted with various substituents including, but not limited to, hydrogen atoms, alkyl, alkenyl, alkynl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. Those of skill in the art will recognize that each of these groups may in turn be substituted. Examples of linkers include, but are not limited to, pH-sensitive linkers, protease cleavable peptide linkers, nuclease sensitive nucleic acid linkers, lipase sensitive lipid linkers, glycosidase sensitive carbohydrate linkers, hypoxia sensitive linkers, photo-cleavable linkers, heat-labile linkers, enzyme cleavable linkers (e.g., esterase cleavable linker), ultrasound-sensitive linkers, and x-ray cleavable linkers

### III. Pharmaceutical Compositions

The nanocarriers can be in a pharmaceutical composition. Pharmaceutical compositions can be for administration by parenteral (intramuscular, intraperitoneal, intravenous (IV) or subcutaneous injection), by instillation, or in a depo, formulated in dosage forms appropriate for each route of administration.

In some embodiments, the compositions are administered systemically, for example, by intravenous or intraperitoneal administration, in an amount effective for delivery of the compositions to targeted cells. Other routes include instillation or mucosal.

In certain embodiments, the compositions are administered locally, for example, by injection directly into a site to be treated. In some embodiments, the compositions are injected or otherwise administered directly to one or more tumors or diseased tissues. Typically, local injection causes an increased localized concentration of the compositions which is greater than that which can be achieved by systemic administration. In some embodiments, the compositions are delivered locally to the appropriate cells by using a catheter or syringe. Other means of delivering such compositions locally to cells include using infusion pumps or incorporating the compositions into polymeric implants which can affect a sustained release of the compositions to the immediate area of the implant.

The compositions can be provided to the cells either directly, such as by contacting it with the cell, or indirectly, such as through the action of any biological process. For example, the compositions can be formulated in a physiologically acceptable carrier or vehicle, and injected into a tissue or fluid surrounding the cell. The compositions can cross the cell membrane by simple diffusion, endocytosis, or by any active or passive transport mechanism.

The selected dosage depends upon the desired therapeutic effect, on the route of administration, and on the duration of the treatment desired. Generally, nanocarrier compositions can be administered in a range of about 0.0001 mg/kg to 100 mg/kg per administration (e.g., daily; or 2, 3, 4, 5 or more times weekly; or 2, 3, 4, 5 or more times a month, etc., as discussed in more detail below). The route of administration can be a consideration in determining dosage as well. For example, in a particular embodiment, a nanocarrier composition is administered in a range of 0.01 mg/kg to 100 mg/kg (e.g., daily; or 2, 3, 4, 5 or more times weekly; or 2, 3, 4, 5 or more times a month, etc., as discussed in more detail below) by intravenous or interpretational routes, or in a range of 0.0001 mg/kg to 1 mg/kg (e.g., daily; or 2, 3, 4, 5 or more times weekly; or 2, 3, 4, 5 or more times a month, etc., as discussed in more detail below) for a subcutaneous route (e.g., local injection into or adjacent to the tumor or tumor microenvironment).

1. Formulations for Parenteral Administration

In a preferred embodiment the compositions are administered in an aqueous solution, by parenteral injection. The formulation can be in the form of a suspension or emulsion. In general, pharmaceutical compositions are provided including effective amounts of one or more active agents optionally include pharmaceutically acceptable diluents, preservatives, solubilizers, emulsifiers, adjuvants and/or carriers. Such compositions can include diluents such as sterile water, buffered saline of various buffer content (e.g., Tris-HCl, acetate, phosphate), pH and ionic strength; and, optionally, additives such as detergents and solubilizing agents (e.g., TWEEN® 20, TWEEN® 80 also referred to as polysorbate 20 or 80), antioxidants (e.g., ascorbic acid, sodium metabisulfite), and preservatives (e.g., Thimersol, benzyl alcohol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and resuspended immediately before use. The formulation may be sterilized by, for example, filtration through a bacteria-retaining filter, by incorporating sterilizing agents into the compositions, by irradiating the compositions, or by heating the compositions.

2. Formulations for Topical or Mucosal Administration

In some embodiments, the compositions are formulated for mucosal administration, for example via pulmonary or intranasal delivery, or topical administration during surgery.

These methods of administration can be made effective by formulating the shell with mucosal transport elements. Compositions can be delivered to the lungs while inhaling and traverse across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art.

Formulations for administration to the mucosa will typically be spray dried drug particles, which may be incorporated into a tablet, gel, capsule, suspension or emulsion. Standard pharmaceutical excipients are available from any formulator.

Mucosal formulations may include one or more agents for enhancing delivery through the nasal mucosa. Agents for enhancing mucosal delivery are known in the art, see, for example, U.S. Patent Application No. 2009/0252672 to Eddington, and U.S. Patent Application No. 2009/0047234 to Touitou. Acceptable agents include, but are not limited to, chelators of calcium (EDTA), inhibitors of nasal enzymes (boro-leucin, aprotinin), inhibitors of muco-ciliar clearance (preservatives), solubilizers of nasal membrane (cyclodextrin, fatty acids, surfactants) and formation of micelles (surfactants such as bile acids, Laureth 9 and taurodehydrofusidate (STDHF)). Compositions may include one or more absorption enhancers, including surfactants, fatty acids, and chitosan derivatives, which can enhance delivery by modulation of the tight junctions (TJ) (B. J. Aungst, et al., J. Pharm. Sci. 89(4):429-442 (2000)). In general, the optimal absorption enhancer should possess the following qualities: its effect should be reversible, it should provide a rapid permeation enhancing effect on the cellular membrane of the mucosa, and it should be non-cytotoxic at the effective concentration level and without deleterious and/or irreversible effects. Intranasal compositions may be administered using devices known in the art, for example a nebulizer.

3. Formulations for Enteral Administration

Pharmaceutical compositions for oral administration can be liquid or solid. Liquid dosage forms suitable for oral administration include, but are not limited to, pharmaceutically acceptable emulsions, microemulsions, solutions, suspensions, syrups and elixirs. In addition to the encapsulated or unencapsulated compound, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor and sesame oils), glycerol, tetrahydrofuryl alcohol, polyethylene glycols and fatty acid esters of sorbitan and mixtures thereof. Besides inert diluents, the oral compositions can also include adjuvants, wetting agents, emulsifying and suspending agents, sweetening, flavoring and perfuming agents.

Solid dosage forms for oral administration include, but are not limited to, capsules, tablets, caplets, dragees, powders and granules. In such solid dosage forms, the encapsulated or unencapsulated compound is typically mixed with at least one inert, pharmaceutically acceptable excipient or carrier such as sodium citrate or dicalcium phosphate and/or (a) fillers or extenders such as starches, lactose, sucrose, glucose, mannitol and silicic acid, (b) binders such as, for example, carboxymethylcellulose, alginates, gelatin, polyvinylpyrrolidinone, sucrose and acacia, (c) humectants such as glycerol, (d) disintegrating agents such as agar-agar, calcium carbonate, potato or tapioca starch, alginic acid, certain silicates and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay and (i) lubricants such as talc, calcium stearate, magnesium stearate, solid polyethylene glycols, sodium lauryl sulfate and mixtures thereof. In the case of capsules, tablets and pills, the dosage form may also contain buffering agents.

Solid compositions of a similar type may also be employed as fill materials in soft and hard-filled gelatin capsules using such excipients as lactose or milk sugar as well as high molecular weight polyethylene glycols and the like. The solid dosage forms of tablets, dragees, capsules, pills and granules can be prepared with coatings and shells such as enteric coatings and other coatings well known in the pharmaceutical formulating art, which can confer enteric protection or enhanced delivery through the GI tract, including the intestinal epithelia and mucosa (see Samstein, et al. Biomaterials. 0.29(6):703-8 (2008).

### IV. Methods of Use

Nanocarrier compositions (including conjugates or conjugates incorporated into nanocarriers) can be administered to a subject in need thereof. The compositions can be used for imaging and diagnostics, for therapeutic or prophylactic applications including delivery of therapeutic agents and gene therapy. In the most preferred embodiments, an active agent is also in or on the nanocarrier. However, in some embodiments, the active agent is free or soluble within the same pharmaceutical formulation as the nanocarrier, or is administered to the subject as part of a separate formulation. The active agent can be, for example, a peptide or protein, a small molecule, or a nucleic acid.

For the individual methods of treatment discussed in more detail below, the active agent is typically selected based on the disease to be treated. The methods typically include administering a subject an effective amount brain targeted nanocarriers including a BBB modulator to increase the permeability of the BBB, and an effective of amount of the active agent to prevent or alleviate one or more symptoms of the disease or condition. In some embodiments, the dosage of the active agent is lower when administered in combination with the BBB modulator-loaded nanocarrier, but can achieve the same or greater effect then when administered absent the BBB modulator-loaded nanocarrier. In some embodiments, the combination of the BBB modulator-load nanocarrier and active agent can achieve a greater effect than when free BBB modulator and active agent administered in combination at the same dosages. In the most preferred embodiments, the BBB modulator and active agent are both encapsulated or dispersed in a nanocarrier, even more preferably the same nanocarrier.

In other embodiments, the active agent is an imaging or diagnostic reagent such as a fluorophore, or a radiotracer.

A. Therapeutic Methods

1. Cancer

In some embodiments, the compositions and methods are used to treat cancer, particularly brain cancer.

In a mature animal, a balance usually is maintained between cell renewal and cell death in most organs and tissues. The various types of mature cells in the body have a given life span; as these cells die, new cells are generated by the proliferation and differentiation of various types of stem cells. Under normal circumstances, the production of new cells is so regulated that the numbers of any particular type of cell remain constant. Occasionally, though, cells arise that are no longer responsive to normal growth-control mechanisms. These cells give rise to clones of cells that can expand to a considerable size, producing a tumor or neoplasm. A tumor that is not capable of indefinite growth and does not invade the healthy surrounding tissue extensively is benign. A tumor that continues to grow and becomes progressively invasive is malignant. The term cancer refers specifically to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site.

The compositions and methods described herein are useful for treating subjects having benign or malignant tumors by delaying or inhibiting the growth of a tumor in a subject, reducing the growth or size of the tumor, inhibiting or reducing metastasis of the tumor; and/or inhibiting or reducing symptoms associated with tumor development or growth. The examples below indicate that the nanocarrier compositions and methods disclosed herein are useful for treating cancer, particular brain tumors, in vivo.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. The compositions are particularly effective in treating carcinomas. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The disclosed methods are particularly useful in treating brain tumors, including primary brain tumors, secondary brain tumors, and a combination thereof. Brain tumors include all tumors inside the cranium or in the central spinal canal. They are created by an abnormal and uncontrolled cell division, normally either in the brain itself (neurons, glial cells (astrocytes, oligodendrocytes, ependymal cells, myelin-producing Schwann cells, lymphatic tissue, blood vessels), in the cranial nerves, in the brain envelopes (meninges), skull, pituitary and pineal gland, or spread from cancers primarily located in other organs (metastatic tumors). Examples of brain tumors include, but are not limited to, oligodendroglioma, meningioma, supratentorial ependymona, pineal region tumors, medulloblastoma, cerebellar astrocytoma, infratentorial ependymona, brainstem glioma, schwannomas, pituitary tumors, craniopharyngioma, optic glioma, and astrocytoma.

“Primary” brain tumors originate in the brain and “secondary” (metastatic) brain tumors originate from cancer cells that have migrated from other parts of the body. Secondary brain tumors can also refer to those that originate from brain cells (for example, secondary GBM refers to GBM derived from benign brain tumors). Metastatic tumors refer to those originate from other parts of the body. Primary brain cancer rarely spreads beyond the central nervous system, and death results from uncontrolled tumor growth within the limited space of the skull. Metastatic brain cancer indicates advanced disease and has a poor prognosis. Primary brain tumors can be cancerous or noncancerous. Both types take up space in the brain and may cause serious symptoms (e.g., vision or hearing loss) and complications (e.g., stroke). All cancerous brain tumors are life threatening (malignant) because they have an aggressive and invasive nature. A noncancerous primary brain tumor is life threatening when it compromises vital structures (e.g., an artery). In a particular embodiment, the compositions and methods are used to treat cancer cells or tumors that have metastasized from outside the brain and migrated into the brain. The metastases can originate from vascular cancer such as multiple myeloma, adenocarcinomas or sarcomas, of bone, bladder, brain, breast, cervical, colo-rectal, esophageal, kidney, liver, lung, nasopharangeal, pancreatic, prostate, skin, stomach, and uterine. In some embodiments, the compositions are used to treat multiple cancer types concurrently. The compositions can also be used to treat metastases or tumors at multiple locations.

2. Neurological Diseases, Disorders, and Conditions

The compositions can be administered to subjects with a neurodegenerative disorder, in need of neuroprotection, or a combination thereof. Neurodegeneration refers to the progressive loss of structure or function of neurons, including death of neurons. Neurodegeneration can be caused by a genetic mutation or mutations; protein misfolding; intracellular mechanisms such as dysregulated protein degradation pathways, membrane damage, mitochondrial dysfunction, or defects in axonal transport; defects in programmed cell death mechanisms including apoptosis, autophagy, cytoplasmic cell death; and combinations thereof. More specific mechanisms common to neurodegenerative disorders include, for example, oxidative stress, mitochondrial dysfunction, excitotoxicity, inflammatory changes, iron accumulation, and/or protein aggregation. Therefore, in some embodiments, the compositions are administered to a subject in need thereof in an effective amount to reduce or prevent one or more mechanisms that cause neurodegeneration.

In particular embodiments, the compositions and methods are used for treating stroke, traumatic brain injury, or epilepsy. Stroke (also cerebrovascular accident (CVA), or cerebrovascular insult (CVI)) occurs when poor blood flow to the brain causes cell death. The two major types of stroke are ischemic due to lack of blood flow and hemorrhagic due to bleeding. Symptoms can include problems understanding or speaking, vertigo, an inability to move or feel on one side of the body, and/or vision loss. In some embodiments the compositions and methods are employed in an effective amount to treat or prevent stroke and/or ischemia by, for example, increasing delivery of an active agent to the brain that increases blood flow in the brain, reduces coagulation (e.g., with anticoagulants), induces arterial dilation, or induces or increases thrombolysis (e.g., with recombinant tissue plasminogen activator (rtPA).

Epilepsy refers to a range of brain-related disorders wherein the normal pattern of neuronal activity becomes disturbed, causing strange sensations, emotions, and behavior or sometimes convulsions, muscle spasms, and loss of consciousness (e.g., seizures). In some embodiments the compositions and methods are employed in an effective amount to treat or prevent epilepsy by, for example, increasing delivery of an active agent to the brain that prevents or reduces seizures, for example, phenytoin, carbamazepine, lamotrigine, levetiracetam, ethosuximide, valproate, phenobarbital, or other anticonvulsant.

In another particular embodiment, the compositions are used to treat a subject suffering from traumatic brain injury (TBI). Traumatic brain injury occurs when an external mechanical force, typically head trauma, causes brain dysfunction.

In some embodiments, the compositions and methods are employed in an effective amount to treat or prevent traumatic brain injury. Traumatic brain injury can have wide-ranging physical and psychological effects. Some signs or symptoms may appear immediately after the traumatic event, while others may not appear until days or weeks later. Symptoms of TBI include, but are not limited to, loss of consciousness; a state of being dazed, confused or disoriented; memory or concentration problems; headache, dizziness or loss of balance; nausea or vomiting; sensory problems such as blurred vision, ringing in the ears or a bad taste in the mouth; sensitivity to light or sound; mood changes or mood swings; feeling depressed or anxious; agitation, combativeness or other unusual behavior; slurred speech; weakness or numbness in fingers and toes; loss of coordination; convulsions or seizures, dilation of one or both pupils of the eyes; and/or clear fluids draining from the nose or ears. In children, additional symptoms include change in eating or nursing habits; persistent crying and inability to be consoled; unusual or easy irritability; change in ability to pay attention; sad or depressed mood; and/or loss of interest in favorite toys or activities.

TBI can be diagnosed using the Glasgow Coma Scale, a 15-point test that helps a doctor or other emergency medical personnel assess the initial severity of a brain injury by checking a person's ability to follow directions and move their eyes and limbs. The coherence of speech also provides important clues. Abilities are scored numerically with higher scores indicating more mild injury. Imaging such as computerized tomography (CT) and magnetic resonance imaging (MRI), as well as intracranial pressure monitoring can also be used to assist in the diagnoses by helping to identify the local(s) and extent of the trauma.

Conventional treatments for TBI include administration of agents such as diuretics, anti-seizer drugs, and coma-inducing drugs; surgery to remove clotted blood, repair skull fractures, and/or relieve pressure inside the skull.

In some embodiments, the compositions are administered in an effective amount to increase neuroprotection, neurorecovery, neurorescue or neuroregeneration in a subject in need thereof. Neuroprotection refers to the relative preservation of neuronal structure and/or function. In the case of an ongoing neurodegenerative insult the relative preservation of neuronal integrity can be measured as a reduction in the rate of neuronal loss over time, which can be expressed as a differential equation (Casson, et al., Clin. Experiment. Ophthalmol., 40 (4): 350-7 (2012)).

Neuroprotective approaches can be used to treat many central nervous system (CNS) disorders including neurodegenerative diseases, stroke, traumatic brain injury, and spinal cord injury. Neuroprotection aims to prevent or slow disease progression and secondary injuries by halting or at least slowing the loss of neurons. Despite differences in symptoms or injuries associated with CNS disorders, many of the mechanisms behind neurodegeneration (discussed above) are the same.

a. Subjects

The methods disclosed herein can be used to treat subjects with a neurological disorder, a psychiatric disorder, a mental illness, neurodegenerative disease or disorder, or a subject in need of neuroprotection. Exemplary neurodegenerative diseases include, but are not limited to, Huntington's Disease (HD), Amyotrophic Lateral Sclerosis (ALS), Parkinson's Disease (PD) and PD-related disorders, Alzheimer's Disease (AD) and other dementias, Prion Diseases such as Creutzfeldt-Jakob Disease, Corticobasal Degeneration, Frontotemporal Dementia, HIV-Related Cognitive Impairment, Mild Cognitive Impairment, Motor Neuron Diseases (MND), Spinocerebellar Ataxia (SCA), Spinal Muscular Atrophy (SMA), Friedreich's Ataxia, Lewy Body Disease, Alpers' Disease, Batten Disease, Cerebro-Oculo-Facio-Skeletal Syndrome, Corticobasal Degeneration, Gerstmann-Straussler-Scheinker Disease, Kuru, Leigh's Disease, Monomelic Amyotrophy, Multiple System Atrophy, Multiple System Atrophy With Orthostatic Hypotension (Shy-Drager Syndrome), Multiple Sclerosis (MS), Neurodegeneration with Brain Iron Accumulation, Opsoclonus Myoclonus, Posterior Cortical Atrophy, Primary Progressive Aphasia, Progressive Supranuclear Palsy, Vascular Dementia, Progressive Multifocal Leukoencephalopathy, Dementia with Lewy Bodies, Lacunar syndromes, Hydrocephalus, Wernicke-Korsakoff's syndrome, post-encephalitic dementia, cancer and chemotherapy-associated cognitive impairment and dementia, and depression-induced dementia and pseudodementia.

Exemplary conditions or subjects that may benefit from or be in need of neuroprotection include, but are not limited to, subjects having had, subjects with, or subjects likely to develop or suffer from a neurodegenerative disease, a stroke, a traumatic brain injury, a spinal cord injury, Post-Traumatic Stress syndrome, or a combination thereof.

b. Symptoms

In some embodiments, the compositions are administered in an effective amount to reduce, alleviate, or prevent one or more other clinical symptoms associated with a neurological disorder, a psychiatric disorder, a mental illness, a neurodegenerative disease, or a central nervous system disorder. Symptoms for these conditions are known in the art and vary from disorder to disorder. For example, common symptoms of neurological disorders include paralysis, muscle weakness, poor coordination, loss of sensation, seizures, confusion, pain and altered levels of consciousness. Similarly, neurodegenerative diseases typically affect one or more body activities including balance, movement, talking, breathing, and heart function.

In some embodiments, the subject has been medically diagnosed as having a neurodegenerative disease or a condition in need of neuroprotection by exhibiting clinical (e.g., physical) symptoms of the disease. In some embodiments, the compositions are administered prior to a clinical diagnosis of a disease or condition. In some embodiments, a genetic test indicates that the subject has one or more genetic mutations associated with a neurodegenerative disease or central nervous system disorder.

Neurodegenerative diseases are typically more common in aged individuals. Therefore in some embodiments, the subject is greater the 40, 45, 50, 55, 60, 65, 70, 75, 80, 85, 90, 95, 100 years in age.

B. Imaging

The Examples below show that the disclosed nanocarrier compositions are particularly effective for brain imaging and diagnostics.

Imaging agents allow for the detection, imaging, monitoring of the presence, progression of a condition, pathological disorder or disease, or any combination thereof. Typically, an imaging agent is administered to a subject in order to provide information relating to at least a portion of the subject (e.g., human). In some cases, an imaging agent may be used to highlight a specific area of a subject, rendering organs, blood vessels, tissues, and/or other portions more detectable and more clearly imaged. By increasing the detectability and/or image quality of the object being studied, the presence and extent of disease and/or injury can be determined.

In some embodiments, the nanocarriers are utilized in methods of imaging. Imaging agents be incorporated in, or attached to, the polymers described herein in the manner discussed below or otherwise known in the art. The methods can includes administering nanocarriers including an imaging agent to a subject, and imaging a region of the subject that is of interest. Although the region of interest for imaging using the disclosed nanocarriers is most typically the brain, other regions of interest may include, but are not limited to, the heart, cardiovascular system, cardiac vessels, blood vessels (e.g., arteries, veins) brain, and other organs. A parameter of interest, such as blood flow, cardiac wall motion, etc., can be imaged and detected using methods and/or systems none in the art. In some embodiments, a method of imaging includes (a) administering to a subject a nanocarrier that includes an imaging agent, and (b) acquiring at least one image of at least a portion of the subject. Suitable systems for imaging include, but are not limited to, magnetic resonance imaging (MRI), positron emission tomography (PET), single-photon emission computed tomography (SPECT) and optical imaging (OI).

In some embodiments, positron emission tomography (PET) is utilized for visualizing the distribution of the imaging agent within at least a portion of the subject. As will be understood by those of ordinary skill in the art, imaging may include full body imaging of a subject, or imaging of a specific body region or tissue of the subject that is of interest (e.g., the brain). In some embodiments, a method may include diagnosing or assisting in diagnosing a disease or condition, assessing efficacy of treatment of a disease or condition, or imaging in a subject with a known or suspected disease or condition. A disease can be, for example, any disease or condition of the brain.

Non-limiting examples of imaging moieties that can be used in imaging agents include ¹¹C, ¹³N, ¹⁸F, ⁷⁶Br, ¹²³I, ¹²⁴I, ¹²⁵I, ¹³¹I, ⁹⁵mTc, ⁹⁵Tc, ¹¹¹In, ⁶²Cu, ⁶⁴Cu, ⁶⁷Ga, and ⁶⁸Ga. In some embodiments, the imaging moiety is selected from the group consisting of ¹⁸F, ⁷⁶Br, ¹²⁴I, ¹³¹I, ⁶⁴Cu, ⁸⁹Zr, ⁹⁹mTc, and ¹¹¹In. The imaging moiety can directly associated (i.e., through a covalent bond) with the nanocarrier, or can be part of another molecule that is incorporate onto or into the nanocarrier. Therefore, in some embodiment, the imaging agent covalently attached to the nanocarrier, and in some embodiments it is not. In some embodiments, a composition including imaging agents or a plurality of imaging agents is referred to as being enriched with an isotope such as a radioisotope. In such a case, the composition or the plurality may be referred to as being “isotopically enriched.” As an example, an “isotopically enriched” composition refers to a composition including a percentage of one or more isotopes of an element that is more than the naturally occurring percentage of that isotope. For example, a composition that is isotopically enriched with a fluoride species may be “isotopically enriched” with fluorine-18 (¹⁸F). Thus, with regard to a plurality of compounds, when a particular atomic position is designated as ¹⁸F, it is to be understood that the abundance (or frequency) of ¹⁸F at that position (in the plurality) is greater than the natural abundance (or frequency) of ¹⁸F, which is essentially zero.

Other specific examples include, but are not limited to, Gadolinium (contrast agent that may be given during MRI scans; highlights areas of tumor or inflammation); PET and Nuclear Medicine Imaging Agents, such as 64Cu-ATSM (64Cu diacetyl-bis(N4-methylthiosemicarbazone), FDG (18F-fluorodeoxyglucose, radioactive sugar molecule, that, when used with PET imaging, produces images that show the metabolic activity of tissues); 18F-fluoride (imaging agent for PET imaging of new bone formation); FLT (3′-deoxy-3′-[18F]fluorothymidine, radiolabeled imaging agent that is being investigated in PET imaging for its ability to detect growth in a primary tumor); FMISO (18F-fluoromisonidazole, imaging agent used with PET imaging that can identify hypoxia (low oxygen) in tissues); Gallium (attaches to areas of inflammation, such as infection and also attaches to areas of rapid cell division, such as cancer cells); Technetium-99m (radiolabel many different common radiopharmaceuticals; used most often in bone and heart scans); Thallium (radioactive tracer typically used to examine heart blood flow); and combinations thereof.

### EXAMPLES

### Example 1: Synthesis of Solid Poly(Amine-Co-Ester) Terpolymers and Terpolymeric NPs

Materials and Methods

Materials

12-dodecanolide (DDL, 98%), 15-pentadecalactone (PDL, 98%), 16-hexadecanolide (HDL, 97%), diethyl sebacate (DES, 98%), N-methyldiethanolamine (MDEA, 99+%), diphenyl ether (99%), Candida Antarctica lipase B (CALB), poly(vinyl alcohol) (PVA, 87-90% hydrolyzed, average molecular weight 30,000-70,000), and branched polyethylenimine (PEI) (25 kDa) were purchased from Aldrich Chemical Co. p-Maleimidophenyl isocyanate (PMPI) was obtained from Pierce Chemical Co., Rockford, Ill. The lipase catalyst was dried at 50° C. under 2.0 mmHg for 20 h prior to use. Other reagents, if not specified, were purchased from Sigma-Aldrich. Luciferase expression plasmid, pGL4.13, was purchased from Promega. RFP expression plasmid, pPRIME-CMV-dsRed, was a gift from Stephen Elledge (Addgene plasmid #11658) (Stegmeier, F., et al., Proc Natl Acad Sci USA, 102: 13212-13217, doi:10.1073/pnas.0506306102 (2005)). TRAIL expression plasmid, pEGFP-TRAIL, was a gift from Bingliang Fang (Addgene plasmid #10953) (Kagawa, S. et al., Cancer Research, 61:3330-3338 (2001)). Mouse B7-1 expression plasmid, pcDNA3-mB7-1 was a gift from Lieping Chen at Yale. mHph2 (YARVRRRGPRRHHHHHHHHHHC (SEQ ID NO:1)) was synthesized by Anaspec.

Statistical Analysis

Differences in different groups were compared using the unpaired t-test or the Mann-Whitney rank-sum test using Prism (version 6.0). Kaplan-Meier analysis was performed to determine survival benefit. p<0.05 was considered a significant difference.

Synthesis of Functionalized Terpolymers

Solid terpolymers (268.6 mg) were dried under vacuum and dissolved under nitrogen in 6.2 mL anhydrous DCM at 45° C., after which 20 μL catalyst dibutyltin dilaurate was added at a concentration of 6.75 mM. PMPI (12 mg) in 1.2 mL DMSO was added with a molar ratio of 0.2 of terpolymers to PMPI. The reaction was conducted in the dark for 12 h. The product was precipitated using 70 mL cold ethanol, washed several times with ethanol to remove any residual traces of DMSO and catalyst, dried at room temperature under nitrogen, and stored as a yellow powder at −20° C.

Synthesis and Purification of Solid Lactone-DES-MDEA Terpolymers

The copolymerization of lactone with DES and MDEA was performed in diphenyl ether using a parallel synthesizer connected to a vacuum line with the vacuum (±0.2 mmHg) controlled by a digital vacuum regulator. In a typical experiment, reaction mixtures were prepared; which contained three monomers (lactone, DES, and MDEA), Novozym 435 catalyst (10 wt % vs. total monomer), and diphenyl ether solvent (200 wt % vs. total monomer). The copolymerization reactions were carried out at a constant temperature in two stages: first stage oligomerization, followed by second stage polymerization. The reaction temperature was set at 90° C. for the copolymerization of all lactones (DDL, PDL, and HDL) with DES and MDEA. During the first stage reaction, the reaction mixtures were stirred under 1 atm of nitrogen gas, after which the reaction pressure was reduced to 1.6 mmHg and the reactions were continued for an additional 72 h. The terpolymers were isolated and purified by first dissolving the crude product mixtures in chloroform. The resultant polymer solutions were then filtered to remove the enzyme catalyst. After being concentrated under vacuum, the filtrates were added dropwise to stirring methanol to cause precipitation of the terpolymers. The obtained white solid polymers were subsequently washed with methanol three times and dried at 40° C. under high vacuum (1.0 mmHg) for 16 h. The isolated yields are reported in Supplementary Table 1.

Structural Characterization of Solid Lactone-DES-MDEA Terpolymers

The composition, molecular weight (M,), polydispersity (M_{w}/Mₙ), and nitrogen content of all solid terpolymers are reported in Table 1. The structure and composition were determined by ¹H NMR spectra, which were recorded on an Agilent DD2 400 MHz NMR Spectrometer (Autosampler). The ¹H NMR spectra showed that the copolymers contained three different types of repeating units: lactone, MDEA, and DES. The molar ratios of lactone to MDEA to DES units were calculated from proton resonance absorptions: number of lactone units from methylene absorption at 4.05 (±0.01) ppm, number of MDEA units from absorption at 4.15 (±0.01) or 2.68 (±0.01) ppm, and number of DES units from absorption at 1.60 (±0.01) ppm after subtracting contribution from lactone units. The M_{w} and Mₙ of polymers were measured by gel permeation chromatography (GPC) using a Waters HPLC system equipped with a model 1515 isocratic pump, a 717 plus autosampler, and a 2414 refractive index detector with Waters Styragel columns HT6E and HT2 in series. Empower II GPC software was used to run the GPC instrument and for calculations. Both the Styragel columns and the RI detector were heated and maintained at 40° C. during sample analysis. Chloroform was used as the eluent at a flow rate of 1 mL/min. Sample concentrations of 2 mg/mL and injection volumes of 100 μL were used. Polymer molecular weights were determined based on a conventional calibration curve generated by narrow polydispersity polystyrene standards from Aldrich Chemical Co. DDL-DES-MDEA (I): ¹H NMR (CDCl₃; ppm) 1.27-1.29 (br.), 1.61 (m, br.). 2.26-2.31 (m), 2.34 (s), 2.69 (t), 4.05 (t), 4.16 (t). PDL-DES-MDEA (II): ¹HNMR (CDCl₃; ppm) 1.26-1.29 (br), 1.61 (m, br), 2.26-2.32 (m), 2.34 (s), 2.69 (t), 4.05 (t), 4.16 (t), plus a small absorption (triplet) at 3.57 ppm due to —CH₂CH₂OH end groups. HDL-DES-MDEA (IV): ¹H NMR (CDCl₃; ppm) 1.26-1.29 (br.), 1.60 (m, br.), 2.25-2.31 (m), 2.32 (s), 2.68 (t), 4.05 (t), 4.15 (t).

Scanning Electron Microscopy (SEM)

The morphology and size were characterized using SEM and ImageJ, respectively. Briefly, samples were mounted on carbon tape and sputter-coated with gold, under vacuum, in an argon atmosphere, using a sputter current of 40 mA (Dynavac Mini Coater, Dynavac, USA). SEM imaging was carried out with a Philips XL30 SEM using a LaB electron gun with an accelerating voltage of 3 kV. The mean particle diameter and size distribution of the NPs were determined by image analysis of particles using image analysis software (ImageJ, National Institute of Health). These micrographs were also used to assess particle morphology.

In Vitro Cytotoxicity Evaluation

HEK293 cells in a 96-well plate were treated with blank NPs to evaluate cytotoxicity of the terpolymers. Cells treated with PEI in the same concentration to that of terpolymers were setup as a control. The cells were incubated with terpolymers or PEI for 72 h. Cell proliferation was then quantified using the standard dimethyl thiazolyl diphenyl tetrazolium salt (MTT) assay. Briefly, 10 mg/mL MTT in PBS was added to the cells making the final MTT concentration at 1 mg/mL, which were incubated for an additional 4 h at 37° C. Afterward, the media was removed and 150 μL DMSO was added to each well to dissolve the formazan crystals. The absorption was measured at 570 nm using a BioTek Instrument ELx800 microplate reader. Each sample was prepared in triplicate and the data was reported as mean±SD. The percentage cell viability of each sample was determined relative to the control (untreated) cells. The effect of mHph2 modification of III-62% NPs on HEK293 cell proliferation was also compared with PEI/pGL4.13 polyplexes and quantified by the MTT assay. For cytotoxicity of pB7-1-loaded ABTT NPs on GL261 cells, GL261 cells were seeded at a density of 5×10³ cells/well in 96-well plates 24 h before transfection. Then pB7-1-loaded ABTT NPs were added to cells and incubated with cells for 72 h. The effect on cell proliferation was quantified using MTT assay and compared with PEI/pB7-1 polyplexes.

Results

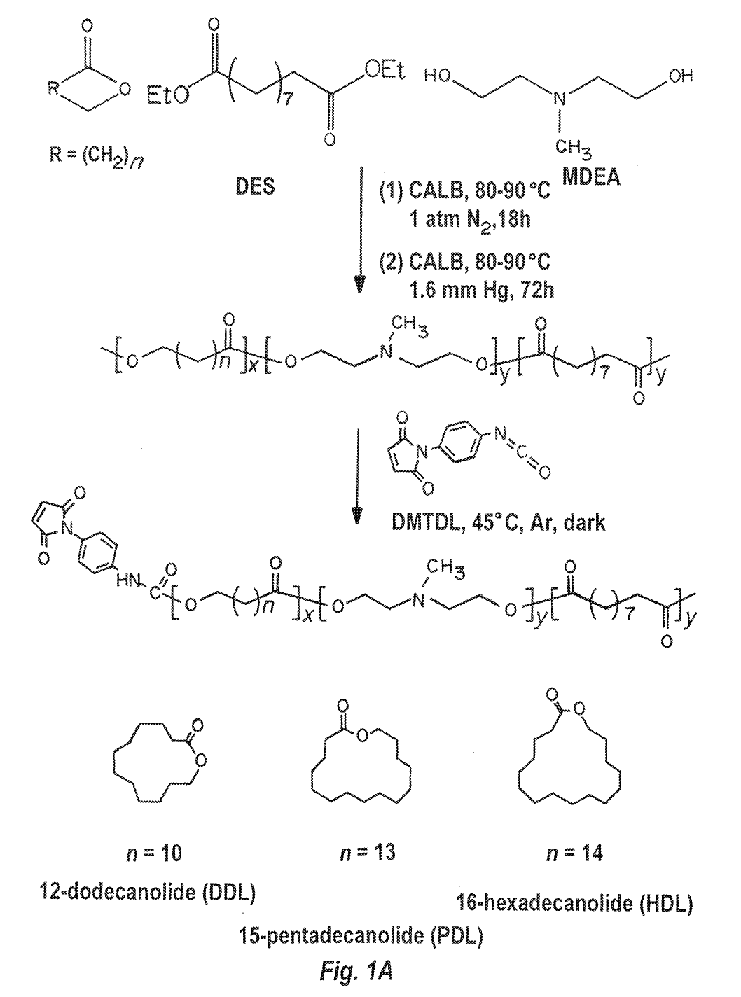

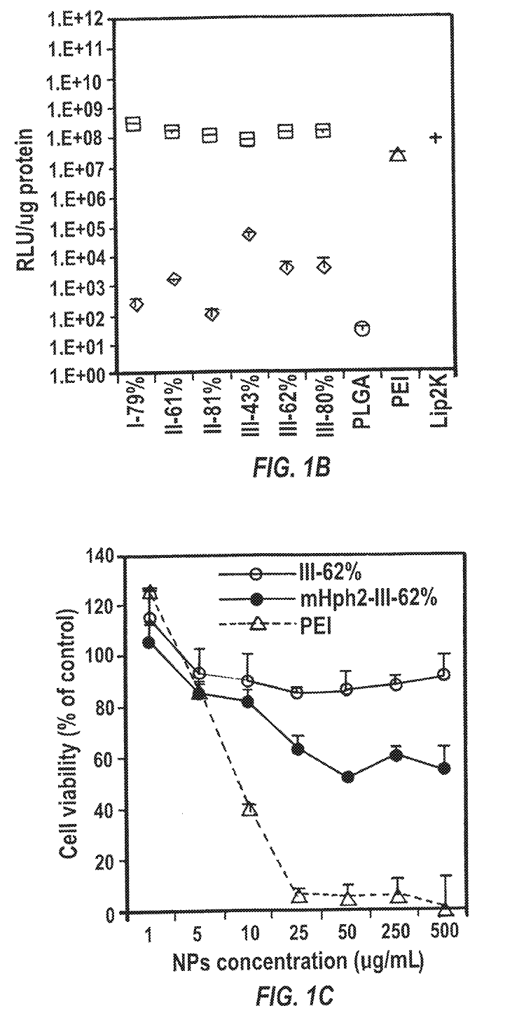

Enzyme-catalyzed chemistry for polymerization of diethyl sebacate (DES) and N-methyldiethanolamine (MDEA) with lactones was developed and it was demonstrated that the resulting liquid poly(amine-co-ester) terpolymers were able to condense genetic material to form polyplexes for efficient gene delivery (Zhou J, et al., Nat Mater, 11(1):82-90 (2012)). Unfortunately, these liquid polyplexes were not sufficiently stable in circulation in vivo for gene delivery to brain tumors. To overcome this problem, the chemistry tuned and solid terpolymers were synthesized by incorporating a high content (40-80%) of lactones (FIG. 1A). Consistent with previous reports (Zhou, Nat Mater, 11(1): 82-90 (2012), Voevodina, et al., Rsc Adv, 4(18): 8953-8961 (2014), the resulting terpolymers with 61-79% dodecanolide (DDL), 45-81% pentadecalactone (PDL), and 43-80% hexadecanolide (HDL), were solid at room temperature.

Table 1 summarizes the synthesis and characteristics of the resulting solid terpolymers, including yield, composition, molecular weight, polydispersity, and nitrogen content. To simplify nomenclature, DDL, PDL, and HDL terpolymers were designated as I, II, and III, respectively. The composition of each individual terpolymer was further denoted as x % lactone indicating the lactone unit content [mol % vs. (lactone+sebacate) units] in the polymer. For example, 11-61% and 111-80% represent terpolymers with 61% PDL and 80% HDL, respectively.

Solid terpolymers were evaluated for synthesis of NPs using the standard emulsion solvent evaporation technique. All terpolymers except I-61% and 11-45% formed spherical NPs. The morphology and size distribution of NPs depended on the ring size and content of lactones: larger ring size or higher lactone content yielded more spherical morphology. For example, while I-61% did not form NPs, I-79%, II-61% and III-62% formed spherical NPs with sizes of 186 nm, 174 nm, and 160 nm, respectively.

TABLE 1
Characterization of solid lactone-DES-MDEA terpolymers
Table S1 Characterization of purified solid lactons-DES-MDEA terpolymers.

| | | Lactone/DES/MDEA | | | | Nitrogen |
|---|---|---|---|---|---|---|
| | Lactone/DES/MDEA | (unit molar | Isolated | | | content |
| Name^{a} | (feed molar ratio) | ratio)^{b} | yield (%) | Mw^{c} | Mw/Mn^{c} | (wt %) |
| I-61% | 60:40:40 | 61:39:39 | 84 | 47400 | 2.1 | 2.36 |
| I-79% | 80:20:20 | 79:21:21 | 87 | 51100 | 3.3 | 1.36 |
| II-45% | 40:60:60 | 45:55:55 | 85 | 17100 | 2.1 | 2.90 |
| II-61% | 60:40:40 | 61:39:39 | 83 | 29500 | 2.5 | 2.11 |
| II-91% | 80:20:20 | 81:19:19 | 88 | 51500 | 3.8 | 1.07 |
| III-43% | 40:60:60 | 43:57:57 | 83 | 30200 | 2.4 | 2.93 |
| III-82% | 60:40:40 | 62:38:38 | 88 | 39600 | 2.8 | 2.00 |
| III-80% | 80:20:20 | 80:20:20 | 89 | 54700 | 3.7 | 1.08 |
| PLGA | / | / | / | 3000~6000 | / | 0 |
| PEI | / | / | / | 25000 | 1.5 | 32.56 |
| PAMAM G5 | / | / | / | 28825 | / | 46.67 |

^{a}The polymer names are abbreviated and simplified. Polymers I, II, and III represent DDL-DES-MDEA, PDL-DES-MDEA, and HDL-DES-MDEA terpolymers, respectively. Each polymer is denoted with x % lactone indicating the lactone unit content [mol % vs. (lactone + sebacate) units] in the polymer.
^{b}Measured by ¹H NMR spectroscopy.
^{c}Measured by GPC using narrow polydispersity polystyrene standards.

Terpolymers were synthesized through two-stage chemistry: oligomerization under 1 atmospheric pressure of nitrogen during which the monomers were converted to non-volatile oligomers, followed by polymerization at 1.6 mmHg during which the by-product ethanol was eliminated and polymer chain growth was initiated and accelerated. All terpolymers were obtained in high yield (83-89%). The composition of the terpolymers, which was determined by ¹H NMR, matched the corresponding monomer feed ratio. Molecular weights (M_{w}) of resulting terpolymers ranged from 17100 to 54700 with polydispersity (M_{w}/Mₙ) between 2.1 and 3.8. These solid terpolymers showed lower nitrogen content ranging from 1.1-2.9 wt % than PEI (32.6 wt %) and polyamidoamine G5 dendrimer (46.7 wt %).

### Example 2: Terpolymeric NPs can Transfect Cells

Materials and Methods

Cell Culture

HEK293 cells, GL261 cells and U87-MG cells were obtained from American Type Culture Collection (ATCC, Rockville, Md., USA). Cells were grown in DMEM medium (Invitrogen) supplemented with 10% fetal bovine serum (FBS, Invitrogen), 100 units/mL penicillin, and 100 μg/mL streptomycin (Invitrogen) in a 37° C. incubator containing 5% CO₂.

In Vitro Gene Transfection

HEK293 cells in 0.25 mL medium in the absence of antibiotics were plated in 48 well plates at a density of 3×10⁴ cells/mL. The plasmid encoding luciferase pGL4.13 (Promega) was used to synthesize solid NPs for evaluating in vitro gene transfection. Transfection using Lipofectamine 2000 (Invitrogen) and PEI followed the standard protocols described in the manufacturer's manual. Briefly, Lipofectamine 2000 was mixed with DNA with the v/m ratio at 2.5 and then incubated at room temperature for 20 min before cell treatment. PEI (1 mg/mL in H₂O) was mixed with DNA with the weight ratio at 3 in serum-free DMEM and vortexed immediately for 10 s. The PEI/DNA polyplexes were then incubated at room temperature for 15 min before cell treatment. For solid NPs, pGL4.13-loaded NPs were suspended in cell culture medium at a concentration of 2 mg/mL. After brief sonication, 0.25 mL of the suspension was added to the cells. Twenty-four hours later, medium with NPs was replaced with fresh cell culture medium. The cells were processed with 125-μL-reporter lysis buffer (Promega). After a freeze-thaw cycle, the cell lysate was collected. After centrifugation at 15,000 rpm for 5 min, 20 μL the supernatant was subjected to luciferase assay using the Luciferase Assay Reagent (Promega) according to the standard protocol described in manufacturer's manual. An additional 20 μL was used to quantify protein content using Pierce BCA protein assay kit (Pierce, Thermo Scientific). The luciferase signal was divided by the amount of total protein for normalization.

Results

HEK293 cells were treated with luciferase gene-loaded NPs and the expression of luciferase was determined two days after treatment. Results shown in FIG. 1B indicated that all tested terpolymeric NPs transfected HEK293 cells. Although the transfection efficiency of unmodified terpolymeric NPs was significantly greater than that of unmodified PLGA NPs, it was lower than leading commercial agents including Lipofectimine 2000 and polyethylenimine (PEI).

To enhance their gene delivery efficiency, the terminal hydroxyl group of the terpolymers using p-maleimidophenyl isocyanate (PMPI) was activated, which converts the hydroxyl group to a maleimide functionality (FIG. 1A). By using III-62% as an example, it was found that with this chemistry, 95% of polymer molecules were functionalized with a maleimide group. When used for particle synthesis, the resulting NPs allowed conjugation of 744 thiolated ligands on their surface. Through a series of optimization studies, it was determined that surface conjugation of peptide mHph2 enhanced the delivery of luciferase gene to HEK293 cells by over 1,000-fold (FIG. 1B). mHph2-modified 111-62% NPs were able to deliver luciferase with efficiency 14.8 and 4.3 fold greater than PEI and Lipofectamine 2000, respectively. Compared to PEI/DNA polyplexes, mHph2-III-62% NPs had significantly lower toxicity (FIG. 1C). Because of their favorable morphology, high efficiency, and low toxicity, mHph2-III-62% NPs were selected for further studies.

### Example 3: NPs can be Targeted to Brain Tumors

### Material and Methods

Preparation of CTX-mHph2-III-62% NPs

One hundred mg mIII-62% in 2 mL DCM was mixed with IR780 iodide (1 mg in 100 μL DMF, infrared fluorescence dye for imaging in vivo distribution). The organic solution was then added drop wise to 4 mL 2.5% PVA under vortex and solicited to form an oil/water emulsion. The emulsion was poured into a beaker containing 0.3% PVA and stirred for 3 h to allow DCM to evaporate and NPs to harden. NPs were collected by centrifugation at 20000 rpm for 30 min. The precipitate was suspended in PBS and reacted first with thiolated CTX (32 μg) for 1 h and then with excess cysteine-terminated peptide mHph2 (4 mg, 0.8 moll) for 1 h at room temperature for conjugation. The unreacted CTX and mHph2 were removed by centrifugation at 20,000 rpm for 30 min and the precipitate was suspended in H₂O and lyophilized for storage and characterization.

In Vivo Distribution of Engineered Terpolymeric NPs

For the evaluation of traditional targeted delivery approach, mice bearing intracranial GL261 tumors were intravenously treated with IR780-loaded mHph2-III-62% NPs or CTX-mHph2-III-62% NPs at a dose of 2 mg NPs/mouse on day 19. Then organs were excised and imaged using an IVIS fluorescence imaging system on day 20.

### Results

mHph2-III-62% NPs were investigated for targeted delivery to brain tumors in vivo. IR780, a near-infrared fluorescent dye that allows for non-invasive detection using an IVIS imaging system, was encapsulated into mHph2-III-62% NPs. Encapsulation of IR780 did not change NP morphology or size. The distribution of IR780-loaded mHph2-III-62% NPs after intravenous administration was evaluated in mice bearing intracranial GL261 gliomas. Low accumulation of NPs was detected in the brain by the IR780 signal. The accumulation of mHph2-III-62% NPs in tumors may have been due to the enhanced permeability and retention (EPR) effect of solid tumors (Smith B R, et al., Nature Nanotechnology, (2014)). However, the signal of NPs in the brain tumor was significantly lower than that in the liver, indicating that mHph2-III-62% NPs had limited efficiency for targeted delivery to brain tumors.

To improve brain tumor-targeting efficiency, mHph2-III-62% NPs, were modified using traditional engineering approaches. By screening a range of ligands, it was found that surface conjugation of chlorotoxin (CTX) enhanced the accumulation of mHph2-III-62% NPs in brain tumors with the greatest efficiency (1.96 fold). CTX is a 36-amino acid peptide with high affinity for matrix metalloproteinase-2 (MMP2), which is preferentially up-regulated in brain tumors but not in the normal brain (Deshane J, et al., J Biol Chem, 278(6):4135-4144 (2003)). It was previously reported that conjugation of CTX enhances drug delivery to intracranial tumors (Veiseh O, et al., Cancer Res, 69(15):6200-6207 (2009)). Nonetheless, despite this improvement, CTX conjugation alone was insufficient, as the signal of NPs in the brain tumor was still significantly lower than that in the liver.

### Example 4: Autocatalytic Brain Tumor-Targeting Delivery as a Novel and Efficient Approach

### Material and Methods

Tumor Model

For the intracranial GL261 tumor implantation, 5-6 week old female C57BL6 mice were anesthetized via intraperitoneal injection of ketamine hydrochloride (75 mg/kg, Abbot Laboratories) and xylazine (7.5 mg/kg, Phoenix Pharmaceutical) in sterile saline. Twenty thousand GL261 cells in 2 μL of PBS were injected into the right striatum (1.8 mm lateral to the bregma and 3 mm of depth) using a stereotactic fixation device with mouse adaptor. For the subcutaneous GL261 xenograft model, 1×10⁶ GL261 cells in 100 μL of PBS were inoculated subcutaneously into the right flank region of female C57BL6 mice. For the intracranial U87-MG glioma model, 1×10⁶ U87-MG cells in 2 μL of PBS was injected into the right striatum of BALB/c nude mice using the same procedures.

Preparation of BBB Modulator-Loaded

CTX-mHph2-III-62% NPs

One hundred mg mIII-62% in 2 mL DCM was mixed with BBB modulatory molecules (2.5 mg Lexiscan, NECA or minoxidil) and IR780 iodide (1 mg in 100 μL DMF) to synthesize the BBB modulator-loaded CTX-mHph2-III-62% NPs.

For the evaluation of BBB-modulator-mediated autocatalytic delivery, mice bearing intracranial GL261 tumors were treated with IR780-labeled CTX-mHph2-III-62% NPs encapsulating Lexiscan, NECA or minoxidil, at a dose of 2 mg NPs/mouse on days 17, 18 and 19. Then the hair on the head was shaved and mice were anesthetized and imaged using the IVIS fluorescence imaging system on day 20. Finally, mice were sacrificed and organs resected for further imaging. CTX-mHph2-III-62% NPs loaded with LEXISCAN® were termed ABTT NPs.

To demonstrate the autocatalytic effect of ABTT NPs, mice bearing intracranial GL261 tumors were intravenously treated with unlabeled CTX-mHph2-III-62% NPs or ABTT NPs on days 17 and 18 at a dose of 2 mg NPs/mouse. Then on day 19, IR780-loaded ABTT NPs were administered to mice at a dose of 2 mg NPs/mouse. At 24 h after the last injection, the hair on the head was shaved and mice were anesthetized and imaged using an IVIS imaging system (Xenogen). Finally, mice were sacrificed and organs resected for further imaging. The fluorescence signal intensity of excised organs was quantified using Living Image 3.0. Mice treated with mHph2-III-62% NPs and IR780-labeled mHph2-III-62% NPs at a dose of 2 mg NPs/mouse were used as controls.

To show that the autocatalytic cumulative effect of ABTT NPs was due to the combination of CTX conjugation and LEXISCAN® encapsulation, mice bearing intracranial GL261 were treated with IR780-loaded mHph2-III-62% NPs or mHph2-III-62%/LEXISCAN® NPs at a dose of 2 mg NPs/mouse on days 17, 18 and 19. Then the hair on the head was shaved and mice were anesthetized and imaged using the IVIS fluorescence imaging system on day 20. Finally, mice were sacrificed and organs resected for further imaging.

To evaluate the kinetics of brain accumulation of ABTT NPs, mice bearing intracranial GL261 tumors were intravenously treated with IR780-loaded ABTT NPs at a dose of 2 mg NPs/mouse on day 19. The hair on mice head was shaved and mice were anesthetized and imaged using an IVIS imaging system at 2 h, 4 h, 6 h, 10 h, and 24 h after treatment. The fluorescence signal intensity in the brain was quantified using Living Image 3.0.

To demonstrate the brain tumor specificity of ABTT NPs, healthy mice were treated with IR780-labeled ABTT NPs at a dose of 2 mg NPs/mouse on days 17, 18 and 19. Then mice were sacrificed and organs resected and imaged on day 20.

To exclude the possibility that ABTT NPs can only be used for the GL261 model, nude mice bearing intracranial luc2-U87-MG glioma were treated with unlabeled CTX-mHph2-III-62% NPs or ABTT NPs on days 17 and 18 at a dose of 2 mg NPs/mouse. On day 19, IR780-loaded ABTT NPs were administered to mice at a dose of 2 mg NPs/mouse. Then mice were anesthetized and imaged using an IVIS imaging system at 4 h, 8 h, and 24 h after treatment. Finally, mice were sacrificed and organs resected for further imaging. Mice treated with CTX-mHph2-III-62% NPs and IR780-labeled CTX-mHph2-III-62% NPs at a dose of 2 mg NPs/mouse were used as controls.

In Vitro LEXISCAN® Release

FITC-labeled LEXISCAN® was used for NP preparation. NPs (3 mg) were suspended in 1 mL PBS 7.4 and incubated with gentle shaking. At each sampling time, NPs were centrifuged for 10 min at 12,000 rpm. The supernatant was collected for quantification of FITC-LEXISCAN® and 1 mL PBS 7.4 was added for continued monitoring of release. Detection of FITC-LEXISCAN® was conducted using the fluorescence reading methods at 495/519 nm.

### Results

The limited enhancement effect of brain tumor targeting by CTX indicated that traditional engineering approaches may be inadequate to overcome the BBB. This finding is consistent with results in the current literature (Gao X, et al., ACS Nano, 8(4):3678-3689 (2014); Huang R, et al., Biomaterials, 32(9):2399-2406 (2011); Huang R, et al., Biomaterials, 32(22):5177-5186 (2011)). To overcome this limitation, autocatalytic mechanism was developed for systemic drug delivery to brain tumors by encapsulating BBB modulators within NPs (FIG. 2A). In this mechanism, a fraction of NPs enter the brain tumor microenvironment through traditional mechanisms. The BBB modulators are then released from the NPs and transiently enhance BBB permeability to more NPs. Through this autocatalytic mechanism, the delivery process creates a positive feedback loop. Consequently, the accumulation efficiency of NPs in the tumor increases with time and subsequent administrations.

To test this mechanism, three well-characterized BBB modulators, LEXISCAN® (Carman A J, et al., The Journal of Neuroscience: The Official Journal of the Society for Neuroscience, 31(37):13272-13280 (2011)), NECA [1-(6-amino-9H-purin-9-yl)-1-deoxy-N-ethyl-β-D-ribofuranuronamide] (Carman A J, et al., The Journal of Neuroscience: The Official Journal of the Society for Neuroscience, 31(37): 13272-13280 (2011)), and minoxidil (Ningaraj N S, et al., Cancer Research, 63(24):8899-8911 (2003)) were selected and evaluated. NECA and LEXISCAN® are adenosine receptor agonists which enhance BBB permeability by decreasing transendothelial electrical resistance, increasing actinomyosin stress fibre formation, and altering tight junction molecules (Carman A J, et al., The Journal of Neuroscience: The Official Journal of the Society for Neuroscience, 31(37):13272-13280 (2011)). Minoxidil is a selective KATP channel agonist that increases the permeability of the BBB in tumors by down-regulating tight junction protein expression (Gu Y-t, et al., Neuropharmacology, 75:407-415 (2013)).

To enable autocatalytic delivery, mice bearing GL261 tumors received three injections daily of NPs co-loaded with IR780 and BBB modulator for three consecutive days. Twenty-four hours after the last injection, both live mice and excised organs were imaged using an IVIS imaging system. LEXISCAN®, NECA, and minoxidil significantly enhanced delivery of CTX-mHph2-III-62% NPs to the brain with comparable efficiency. The signal intensity of NPs in the tumor-bearing right brain surpassed that of all other organs including the liver, kidney, spleen, heart, and lung.

Of the three BBB modulators, LEXISCAN® is currently used in clinic in an intravenous formulation for myocardial perfusion imaging and has a favorable safety profile. Therefore, LEXISCAN® was selected for further studies. Encapsulation of LEXISCAN® did not change the morphology of CTX-mHph2-III-62% NPs, or their ability to transfect cells (FIG. 2B). LEXISCAN® was released from the NPs in a controlled manner (FIG. 2C). In accordance with the proposed mechanism, the tumor accumulation efficiency autocatalytically increased with subsequent administrations: the efficiency was enhanced by 2.26-fold simply by priming mice with two treatments of the same NPs without IR780 (FIG. 2D). With this delivery strategy, the signal intensity of NPs in the brain tumor was 4.3, 4.2, 5.6, 31.7, and 12.7 times greater than that in the liver, spleen, kidney, heart and lung, compared to 0.3, 0.7, 0.9, 9.6, and 1.8 times for mHph2-III-62% NPs (FIG. 2D). These results were likely due to a more than additive effect of tumor targeting by CTX and autocatalysis by Lexiscan, as either modification alone did not enhance NP targeting to this degree. To further simplify the nomenclature, CTX-mHph2-III-62% NPs loaded with LEXISCAN® were referred to as ABTT NPs.

### Example 5: ABTT NPs can be Used for (PET/CT) and High-Resolution Confocal Microscopy Imaging

### Material and Methods

Preparation of F-18 Labeled ABTT and mHph2-III-62% NPs

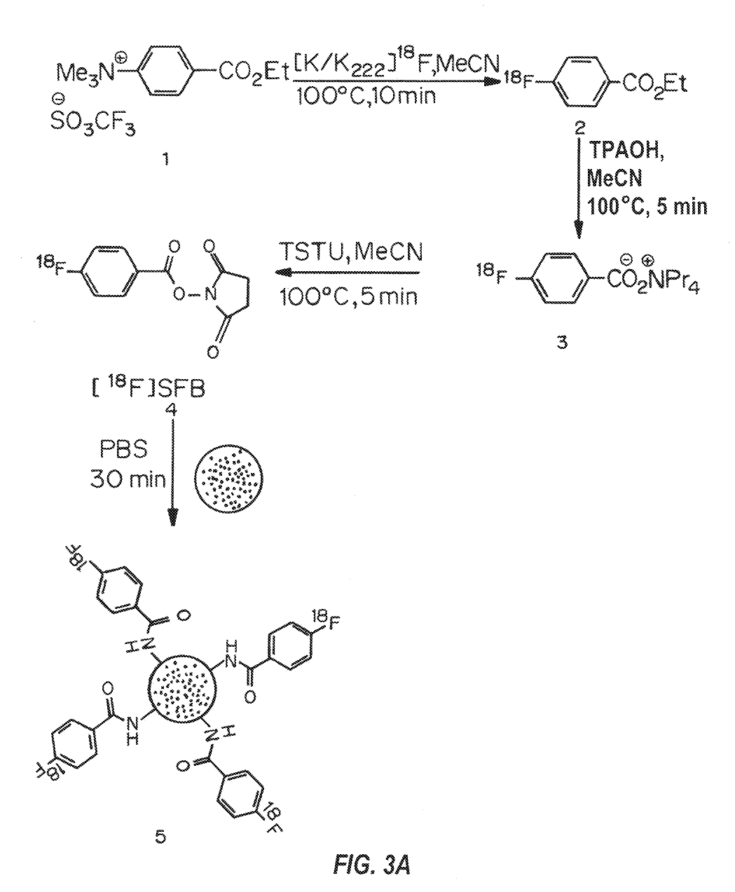

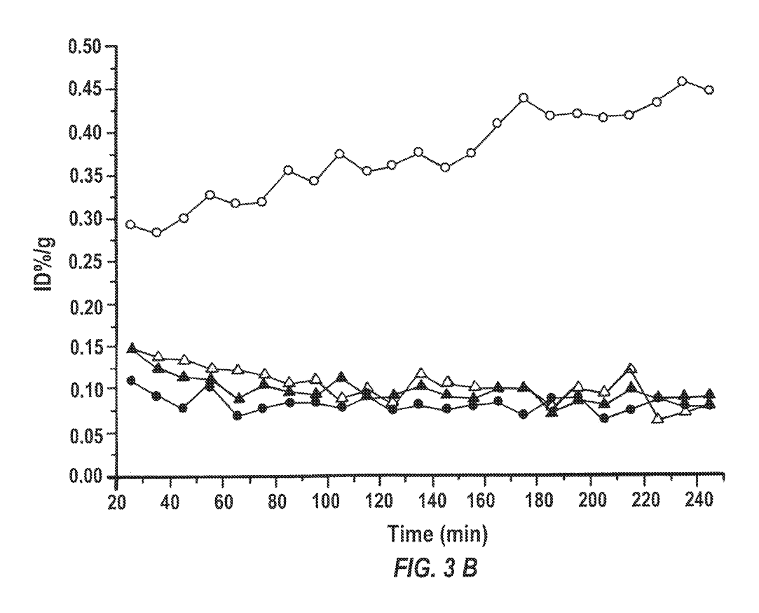

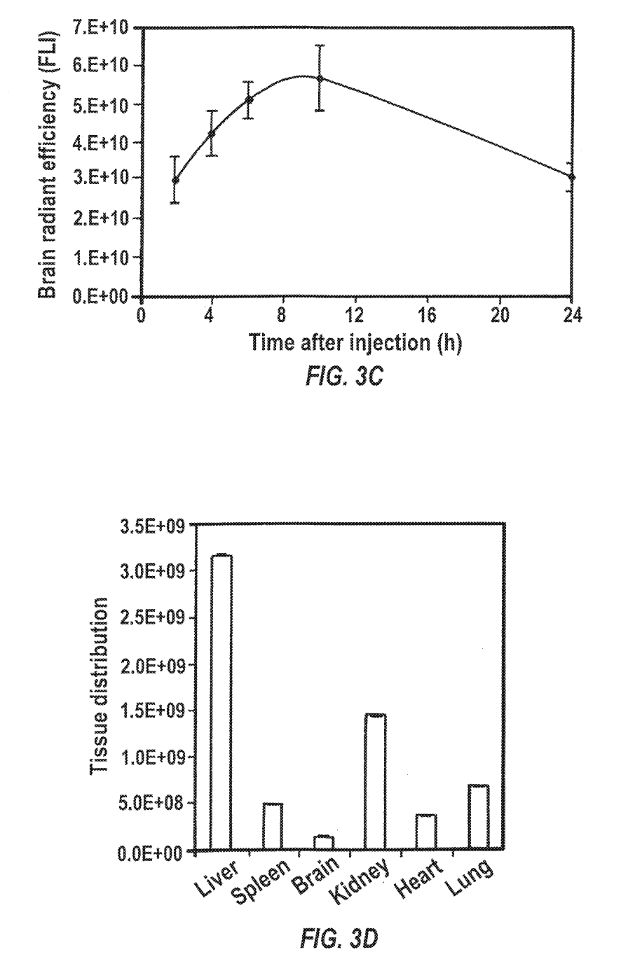

The F-18 radiolabeling of the ABTT and mHph2-III-62% NPs used a common prosthetic group, N-succinimidyl 4-[¹⁸F]fluorobenzoate ([¹⁸F]SFB), reacting with a free amine group on the surface of the NPs. [¹⁸F]SFB was synthesized according to a simplified three-step, one-pot procedure (Tang, G., et al., Journal of Labeled Compounds and Radiopharmaceuticals, 53:543-547 (2010)) with modifications (FIG. 3A). In brief, ethyl 4-(trimethylammonium triflate)benzoate (1±0.3 mg) in anhydrous acetonitrile (0.2 mL) was added to the dried [18F]fluoride/Kryptofix-2.2.2 complex and heated at 100° C. for 10 min. Tetrapropylammonium hydroxide (20 μL, 1 M in water) in acetonitrile (0.2 mL) was then added and heated at 100° C. for 5 min. The resulting solution was first dried with a gentle nitrogen stream at 100° C. and then azeotropically dried with two more portions of anhydrous acetonitrile (0.2 mL). A solution of N,N,N′,N′-tetramethyl-O-(N-succinimidyl)uranium tetrafluoroborate (TSTU; 1±0.3 mg) in anhydrous acetonitrile (0.2 mL) was added to the dried mixture and heated at 100° C. for 5 min. The crude product was diluted with a mixture of 35/65 acetonitrile/0.1% trifluoroacetic acid (1.5 mL) and loaded to a semi-preparative HPLC system for purification (HPLC column: Phenomenex Luna C18, 250×10 mm; mobile phase: 35/65 acetonitrile/0.1% trifluoroacetic acid; flow rate: 5 mL/min.). A radioactive fraction at ˜16 min was collected and diluted with water. The solution was passed through a Waters C18 SepPak. The SepPak was rinsed with water (10 mL), and then eluted with acetonitrile (1 mL) to recover the trapped [¹⁸F]SFB. Analytical HPLC showed that radiochemical purity of [¹⁸F]SFB was great than 99%. The total synthesis time was 70±5 min with decay-uncorrected radiochemical yield of 38±10%? (n=4).

Solution of [¹⁸F]SFB in acetonitrile was dried at 60° C. with a gentle nitrogen stream and the residue re-dissolved with an appropriate amount of 1×PBS solution to result in a strength of 10 mCi/0.5 mL. A portion (0.5 mL) of the solution was added, separately, to ABTT NPs (2 mg) or mHph2-III-62% NPs (2 mg) in a 1.5 mL Eppendorf vial. The mixture was sonicated for 20 min, and then centrifuged at 17,000 rpm for 10 min. The supernatant was removed. The radiolabeled NPs were rinsed with PBS (0.5 mL, twice), re-dissolved in appropriate amount of PBS, vortex and then sonicated for 20 min to form a solution ready for injection (˜0.5 mCi/0.2 mL). The total conjugation and purification time was 60±5 min with a conjugation yield of 51±3% for ABTT NPs (n=3) and 48±5% for mHph2-III-62% NPs (n=3).

PET Imaging Procedures and Imaging Analysis

PET scan and image analysis were carried out using a microPET scanner (Inveon, Siemens Medical Solutions). All pre-primed brain tumor model mice (4 aimed and 4 controls) were injected intravenously with ˜0.5 mCi (0.2 mL) of [¹⁸]-labeled ABTT NPs or mHph2-III-62% NPs while awake. A pair of the mice was then lightly anesthetized and placed on the microPET scanner to first receive a short CT scan. Dynamic PET scans were then acquired for 4 h. PET images were reconstructed using a two-dimensional ordered-subset expectation maximum (OSEM) algorithm with no correction for attenuation or scatter. The left and right brain regions of interest on the PET images were manually drawn based on the merged PET/CT image. Radioactivity within the tumor and the corresponding left hemisphere were obtained from mean pixel values within the multiple ROI volume and then converted to MBq/mL, and standardized to percent injected dose per gram (% ID/g).

High-Resolution Confocal Microscopy Study

Mice containing glioma received four ABTT NP injections. Two days after NP injections, 100 μL of PE Rat Anti-Mouse CD31 antibody (BD Pharmingen #553373) was injected intravenously to label the tumor vasculature. One hour after injecting the antibody, mice were perfused with 1×PBS followed by 4% paraformaldehyde (PFA). Brains were incubated overnight in 4% PFA and 60 μm thick sections were obtained using a vibratome (Leica). Tumor containing brain sections were mounted and used for high-resolution confocal imaging. Leica SP5 confocal microscope with 10× air, 40× and 63× objectives with APO oil immersion were used to obtain Z-stacks at 0.5 μm step sizes and zooms from 1 to 5. Images were processed using NIH ImageJ.

### Results

With the unprecedented efficiency in crossing the BBB to target tumors, ABTT NPs may have the potential to be used for imaging of brain tumors. To demonstrate this feasibility, ABTT NPs and control mHph2-III-62% NPs were labeled with a radioactive tag by reacting the free amine group on the NPs with N-succinimidyl 4-[¹⁸F]-fluorobenzoate (SFB) to form an amide bond (4-[¹⁸F]-fluorobenzamide-NPs) (FIG. 3A). The labeled NPs were administered to GL261 tumor-bearing mice through tail vein injection after first priming with three injections of unlabeled NPs. Immediately after treatment, mice were subjected to PET/CT scanning. The accumulation of NPs in the brain was continuously monitored over four hours. Resulting images were reconstructed using a two-dimensional ordered-subset expectation maximum algorithm without attenuation or scatter correction. The left and right brain regions of interest in the PET images were manually drawn based on merged PET/CT images. The summed PET image (210-240 min), which showed the dynamic growth of the PET signal with time, indicated that ABTT NPs efficiently penetrated the BBB and accumulated in tumors. In contrast, control NPs did not have comparable efficiency.

The radioactivity within the tumor and the corresponding area of the left hemisphere was quantified based on mean pixel values, which was further converted to MBq/mL and standardized to percent of injected dose per gram (% ID/g). The radioactivity within the tumor continuously grew over the entire four hour period. In contrast, the radioactivity within the corresponding left hemisphere remained low over this time window (FIG. 3B). Due to technical reasons, the PET scan was not continued beyond the 4-hour time point. However, according to a separate study in which the kinetics of NP accumulation in brain tumors was measured based on IR780 signal (FIG. 3C), it is believed that the PET signal in the brain tumor would gradually increase and peak between 8 and 12 hours post-treatment.

The specificity and sensitivity of ABTT NPs for brain tumors was investigated. For this purpose, IR780-loaded ABTT NPs was administered to normal mice without tumors. The results in FIG. 3D indicate that ABTT NPs had a limited ability to penetrate the normal BBB, as IR780 signal in the normal brain was undetectable. To further characterize the penetrability of ABTT NPs at the cellular level, the location of ABTT NPs in the brain was examined using a high-resolution confocal microscopy. In this study, mice bearing green fluorescent protein (GFP)-expressing tumor were treated with ABTT NPs encapsulated with DiD, a red fluorescence dye, after which, mice were euthanized, extensively perfused. The brains were sectioned and subjected to microscopic analysis. Consistent with previous findings, ABTT NPs preferentially accumulated in intracranial tumors, but not in the surrounding normal brain tissue. ABTT NPs were homogenously distributed over the entire brain tumor region. A fraction of ABTT NPs were located perivascularly around tumor blood vessels (blue), indicating that they crossed the BBB in the tumors. With further magnification, a cluster of NPs located within a single cell were detected, indicating that ABTT NPs were capable of penetrating cell membrane and entering cellular compartments with high efficiency. Notably, in addition to tremendous specificity, ABTT NPs also demonstrated high sensitivity for tumor cells, as they were able to efficiently accumulate in small distant tumor islands that contained only 10-20 tumor cells.

### Example 6: ABTT NPs are Effective for Systemic Delivery of Brain Cancer Gene Therapy

### Materials and Methods

Synthesis of ABTT NPs

ABTT NPs were synthesized according to standard emulsion procedure (Strohbehn G, et al., Journal of Neuro-oncology, 121(3):441-449 (2015); Zhou J, et al., Proc Natl Acad Sci USA, 110(29):11751-11756 (2013); Zhou J, et al., Biomaterials, 33(2):583-591 (2012)). Briefly, for synthesis of DNA-loaded NPs, 500 μg DNA in 100 μL water was added dropwise to 100 mg mIII-62% in 2 mL DCM containing 2.5 mg LEXISCAN® under vortex. This mixture was sonicated to form a water/oil emulsion (1st emulation). The water/oil emulsion was then added dropwise to 4 mL 2.5% PVA under vortex and sonicated to form a water/oil/water emulsion (2nd emulation). The double emulsion was poured into a beaker containing 0.3% PVA and stirred for 3 h to evaporate DCM. NPs were collected by centrifugation at 20000 rpm for 30 min. The precipitate was suspended in PBS and reacted first with thiolated CTX (32 μg) for 1 h and then with excess cysteine-terminated peptide mHph2 (4 mg, 0.8 μmol) for 1 h at room temperature. The unreacted CTX and mHph2 were removed by centrifugation at 20000 rpm for 30 min and the precipitate was suspended in H₂O and lyophilized for storage and characterization. For synthesis of IR780 or DiD-loaded NPs, the same procedures without the 1st emulation step were used.

In Vitro Gene Transfection

For gene transfection on GL261 cells, GL261 cells were plated in 48-well plates at a density of 5×10⁴ cells/well 24 h before transfection. Then pGL4.13-loaded ABTT NPs were added to cells and incubated for 12 h. At 6 h. 12 h, 24 h, 48 h, and 72 h after transfection, cells were collected and luciferase expression was measured. GL261 cells were plated in 48-well plates at a density of 5×10⁴ cells/mL 24 h before transfection. mHph2-modified NPs and ABTT NPs were given to cells. The luciferase expression at 48 h after treatment was used to evaluate the influence of CTX conjugation and LEXISCAN® encapsulation on gene transfection.

In Vitro Cytotoxicity Evaluation

The general parameters for in vitro cytotoxicity evaluation are discussed above in Example. For cytotoxicity of pB7-1-loaded ABTT NPs on GL261 cells, GL261 cells were seeded at a density of 5×10³ cells/well in 96-well plates 24 h before transfection. Then pB7-1-loaded ABTT NPs were added to cells and incubated with cells for 72 h. The effect on cell proliferation was quantified using MTT assay and compared with PEI/pB7-1 polyplexes.

In Vivo Gene Transfection

The pRFP-loaded ABTT NPs were injected into the tail vein of mice at a dose of 2 mg NPs/mouse. Transfection was conducted for three consecutive days. Two days after the last transfection, animals were euthanized and perfused. The brains were removed, fixed in 4% paraformaldehyde for 48 h. Brains were then placed in 15% sucrose solution until subsidence (6 h), then in 30% sucrose until subsidence (24 h) and finally frozen in OCT embedding medium (Sakura, Torrance, Calif., USA) at −80° C. Frozen sections of 20-μm thickness were prepared with a cryotome Cryostat (Leica, C M 1900, Wetzlar, Germany), stained with 300 nM DAPI for 10 min at room temperature and examined under the fluorescence microscope. For GFP-expressing U87-MG tumors, brains were first imaged using a Maestro™ in-vivo fluorescence imaging system (Cambridge Research & Instrumentation, Inc.) and then embedded with paraffin. Paraffin sections of 5-μm thickness were stained with DAPI and analyzed by fluorescence microscopy.

Therapeutic Evaluation of pB7-1-Loaded ABTT NPs

For evaluation in subcutaneous GL261 tumors, treatments were started when tumor volumes reached ˜50 mm³. Tumor size was measured two times a week using traceable digital venire callipers (Fisher). The tumor volumes were determined by measuring the length (l) and the width (w) and calculating the volume (V=1/2×lw²). For intracranial tumors, treatments were started five days after the tumor cell injection. Injections were performed through the tail vein three days a week for 3 weeks. The animals' weight, grooming, and general health were monitored on a daily basis. Mice were euthanized after either a 15% loss in body weight or when it was humanely necessary due to clinical symptoms.

In Vivo B7-1 Expression and Histological Assessment

To detect B7-1 expression within the tumors, brains were harvested, embedded with paraffin, and sectioned for antibody staining. Histological assessment of intracranial gliomas was carried out by haematoxylin and eosin staining of 5 μm sections, analyzed by microscopy. B7-1 expression was detected with anti-CD80 antibody labeled with Alexi Fluor 647.

Therapeutic Effect of pTRAIL-Loaded ABTT NPs on Intracranial luc2-U87-MG Tumors

Mice with comparable luciferase expression intensity in brain were randomly divided into treatment groups with eight mice per group. Injections were performed through the tail vein three days a week (Monday, Wednesday and Friday) for 3 weeks. The animals' weight, grooming, and general health were monitored on a daily basis. Animals were killed after either a 15% loss in body weight or when it was humanely necessary due to clinical symptoms. The Kaplan-Meier survival curves were plotted. For the two surviving mice, bioluminescence imaging was performed on day 60 to confirm the absence of luc2-U87-MG tumor.

### Results

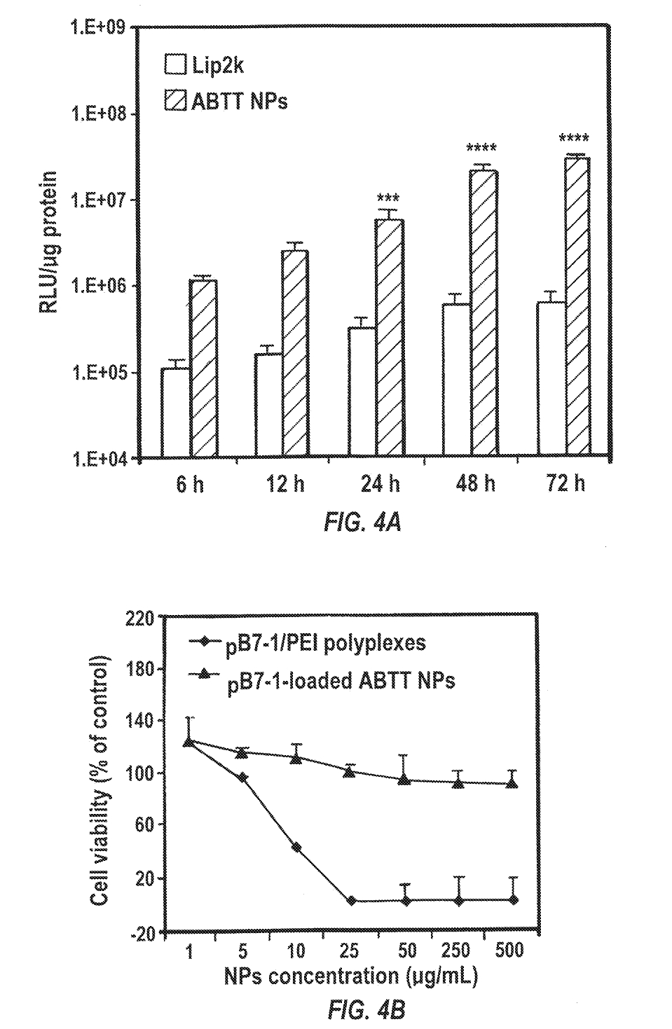

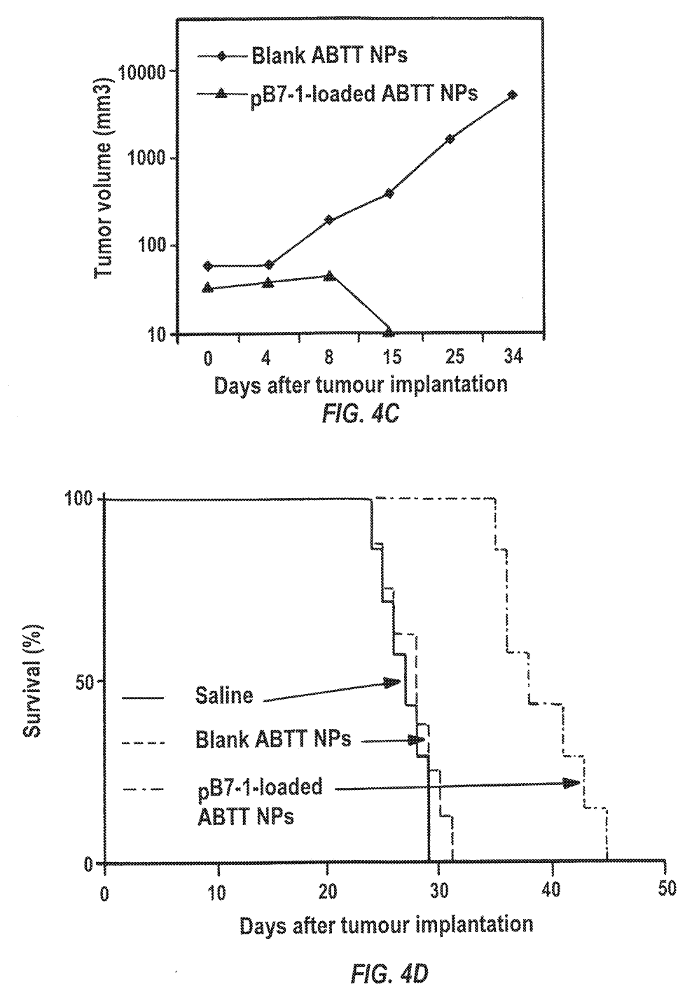

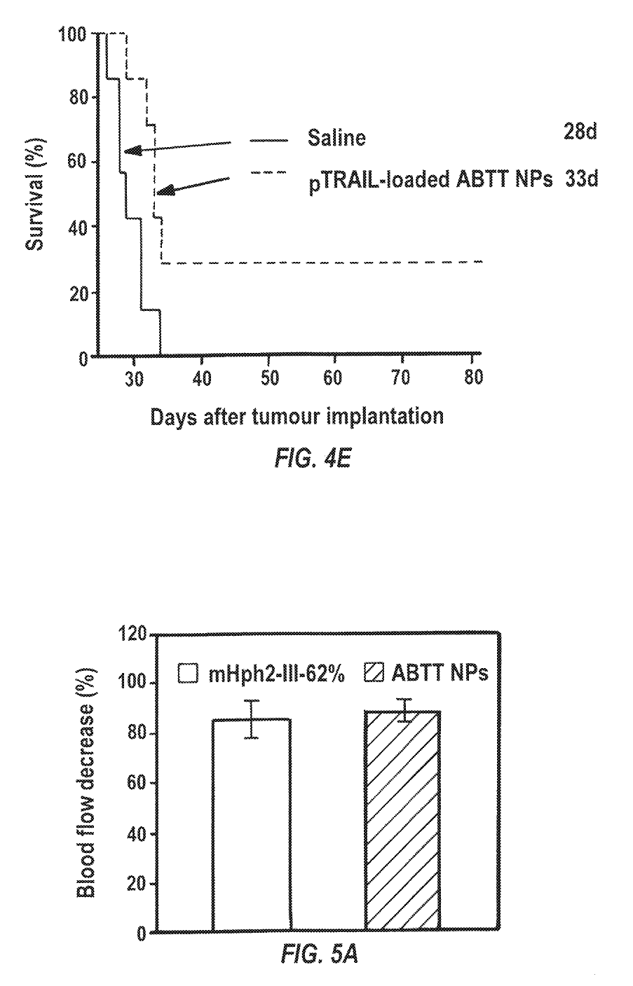

To assess the ability of ABTT NPs to transfect GL261 cells, cells were treated with luciferase plasmid-encapsulated ABTT NPs, which retained their spherical morphology at 161 nm. Luciferase expression was measured at 6, 12, 24, 48, and 72 hours post treatment. The gene transfection efficiency of ABTT NPs was significantly higher than that of Lipofectamine 2000 at all-time points (FIG. 4A). At 72 hours, the luciferase signal in ABTT NP-treated cells was 48.1 times greater than that in Lipofectamine 2000-treated cells (FIG. 4A).

Next the ability of ABTT NPs to transfect intracranial GL261 gliomas in vivo by treatment with NPs encapsulating plasmid DNA for expression of red fluorescence protein (pRFP) was investigated. The results indicated that intravenous administration of pRFP-loaded ABTT NPs efficiently transfected GL261 tumors in the brain, as evident by the strong red fluorescent signal in tumors and its absence in normal brain tissue.

ABTT NPs were also evaluated for systemic delivery of gene therapy to GL261 gliomas. Malignant gliomas often evolve a variety of mechanisms to reduce the expression of B7-1, a costimulatory molecule necessary for T-lymphocyte activation (Chen L, et al., Nature Reviews Immunology, 13(4):227-242 (2013)). Correspondingly, cytotoxic T-lymphocytes fail to recognize and eradicate the tumors (Han S J, et al., Neurosurgery Clinics of North America, 23(3):357-370 (2012); Capece D, et al., Journal of Biomedicine & Biotechnology, 2012:926321 (2012)). Therefore, one potential approach to treat malignant gliomas is to restore the normal function of B7-1 by delivering B7-1 gene directly to tumors. To test this approach and evaluate the use of ABTT NPs for systemic delivery of gene therapy, B7-1 plasmid DNA (pB7-1)-loaded ABTT NPs were administered to intracranial GL261 tumor-bearing mice through tail vein injection and monitored their survival over time. B7-1 gene-loaded ABTT NPs were spherical in morphology with an average diameter of 157 nm. B7-1 gene-loaded ABTT NPs showed minimal cytotoxicity to GL261 cells (FIG. 4B).

Kaplan-Meier analysis revealed that mice treated with B7-1 gene-loaded ABTT NPs had significant improvement in median survival, which was 38 days, compared to 28 and 29 days for mice receiving saline and blank ABTT NPs, respectively (FIG. 4D, p<0.0001 for both comparisons). Successful delivery of B7-1 was confirmed by B7-1 immunostaining. In contrast to blank ABTT-NP-treated tumors, the B7-1-loaded ABTT NP-treated tumors showed significant up-regulation of B7-1. The efficacy of B7-1 gene-loaded ABTT NPs was limited to 10-day survival enhancement, presumably due to an intrinsic limitation of the GL261 intracranial tumor model. T-cells cannot enter the brain unless they are activated. Apparently, intracranial inoculation of GL261 cells does not allow for penetration of an adequate number of T-lymphocytes, as a single intratumoral administration of pB7-1-loaded ABTT NPs eliminated tumors implanted in the flank (FIG. 4C).

ABTT NPs were further investigated for systemic gene therapy in U87-MG-derived human glioma. Consistent with the findings in the GL261 model, intravenous administration of ABTT NPs resulted in preferential accumulation of NPs in tumors with high efficiency. When pRFP was encapsulated, ABTT NPs selectively transfected intracranial tumors. Intravenous administration of tumor necrosis factor-related apoptosis-inducing ligand (TRAIL)-loaded ABTT NPs significantly enhanced tumor-bearing mouse survival (FIG. 4E). In particular, 2 of 6 mice in the treatment group survived over 90 days, during which tumors in the brain were undetectable. Of note, in both therapeutic studies, mice received 9 treatments of NPs at a dose of 2 mg/injection. The maximum tolerable dose for ABTT NPs is greater than 10 mg per treatment. Therefore, it is believed that further enhanced therapeutic benefit can be achieved with more aggressive treatment regimens.

### Example 7: ABTT NPs is a General Platform for Systemic Drug Delivery to Neurological Disorders

### Materials and Methods

ABTT NPs for Systemic Delivery to Cerebral Ischemia

The animals were anesthetized with 5% isoflurane (Aerrane, Baxter, Deerfield, Ill.) in 30% O₂/70% N₂O using the CDS 9000 Tabletop Anaesthesia system (Smiths Medical ASD, Inc., USA) and then the isoflurane was maintained at 1.5%. Body temperature of the mice was maintained during surgery with a heating pad. Mice were placed in the supine position. Fur on the neck region was shaved. The surgical site was disinfected with a Povidone-iodine solution. A 1 cm long midline neck incision was made under a dissecting microscope (Leica A60). Blunt dissection was performed to expose the right common carotid artery (CCA), external carotid artery (ECA), and internal carotid artery (ICA), while preserving the vagus nerve. The CCA was temporarily occluded by a 6-0 silk suture and then the bifurcation CCA was separated. The ECA further dissected distally, coagulated the ECA and its superior thyroid artery (STA) and occipital artery (OA) branches using a coagulator, cut the ECA, OA, and STA at the coagulated segment. Two sutures were placed around the ECA stump, one was permanent knot close to the coagulated segment and the other one was temporary knot that was close to the bifurcation. The ICA was further dissected and then clipped with a microvascular clip. Then a small hole in the ECA between permanent and temporary sutures was made with Vanes-style spring scissors. A 6-0 silicon-coated monofilament suture (Ducal Corporation) was introduced into the ECA and the temporal knot was tightened to prevent bleeding and the microvascular clip on ICA was removed. The monofilament was gently advanced from the lumen of the ECA into the ICA a distance of 8-10 mm beyond the bifurcation to occlude the origin of MCA until the monofilament could not be further advanced. The occlusion lasted 60 min and then the monofilament was withdrawn to allow blood reperfusion. The suture on the ECA was permanently tied off and the temporary suture on the CCA was removed to allow blood recirculation. Blood vessel occlusion was further confirmed by measuring cerebral blood flow using a Doppler blood flowmeter (AD Instruments Inc., Colorado Springs, Colo., USA) for the duration of the surgery.

To determine cerebral blood flow, animals were placed in the supine position, with the head firmly immobilized in a stereotactic frame (Model 900 Small Animal Stereotactic; David Kopf Instruments, Tujunga, CA, USA). A burr hole (1.5 mm diameter) was drilled into the skull using a surgical bone drill system (Microtorque II; Harvard Apparatus, Holliston, Mass., USA) at 5-6 mm lateral and 1-2 mm posterior to the bregma, without injury to the dura mater. The laser Doppler flow probe (Standard Pencil Probe, MNP 100XP; AD Instruments, Inc.) was carefully positioned at the craniotomy site sing a three-way micromanipulator (Narishige International, Inc., East Meadow, NY, USA). The above procedure was carried out prior to blood vessel occlusion to induce ischemia. Cerebral blood flow was continuously monitored (2-Hz sampling rate) from before the onset of ischemia until 5 min after reperfusion. Middle cerebral artery occlusion was confirmed by reduction in the local cerebral blood flow from the baseline value. One hour after ischemia, the intraluminal filament was withdrawn to allow for reperfusion, which was confirmed by restoration of the local cerebral blood flow to baseline. The incision was sutured, and animals were allowed to recover, during which time their bodies were kept warm with a heating lamp. Blood pressure was monitored before, during, and after the release of occlusion (AD Instruments, Inc.). Arterial blood samples were collected from the femoral arterial catheter before, during, and immediately after the release of occlusion to determine levels of blood gases (arterial PO2, arterial PCO2 and pH) (ABL-500; Radiometer, Copenhagen, Denmark) and glucose (Accu-Chek, Roche Diagnostics, Indianapolis, Ind., USA).

Solutions of the IR780-loaded mHph2-III-62% NPs or IR780-loaded ABTT NPs were injected into the tail vein of mice at a dose of 2 mg NPs/mouse/injection at 0 h, and 24 h and 48 h after reperfusion. At 24 h after the last injection, mice were sacrificed and organs resected for imaging using the IVIS fluorescence imaging system. The brains were sliced into 2 mm sections coronally. Each section was stained by immersion in 1% TTC solution and incubated for 20 min. Stained sections were recorded with a camera. In addition, fluorescence signal intensity of excised organs was quantified using Living Image 3.0

ABTT NPs for Systemic Delivery to Traumatic Brain Injury

To produce trauma to the temporal and frontal cortices reproducibly, a pneumatic piston was precisely driven by using miniature precision valves (Clippard, Cincinnati, Ohio) powered by nitrogen. Displacement and velocity of the piston was determined by a digital motion detector (EPD Technologies, Elmsford, NY). Female BALB/c mice were anesthetized with pentobarbital, and their heads were placed securely in a stereotaxic frame. A scalp incision was made to locate the bregma. A burr hole was drilled into the skull using a surgical bone drill system (Microtorque II; Harvard Apparatus, Holliston, Mass., USA) and the bone was removed without trauma to the underlying dura and brain parenchyma. A 3-mm diameter stainless steel piston then was positioned to deliver and impact 2 mm right and 2 mm dorsal to the bregma. Once the piston was activated, the velocity and time of impact was noted, as well as the amount of damage to the skull. The scalp incision was closed by using 6-0 suture.

Solutions of the IR780-loaded mHph2-III-62% NPs or IR780-loaded ABTT NPs were injected into the tail vein of mice at a dose of 2 mg NPs/mouse/injection at 0 h, and 24 h and 48 h after traumatic brain injury. At 24 h after the last injection, mice were sacrificed and organs were resected for imaging using IVIS fluorescence imaging system. In addition, fluorescence signal intensity of excised organs was quantified using Living Image 3.0.

### Results

The ABTT NPs developed in the study above were designed to target MMP2 in the glioma microenvironment via CTX (Deshane J, et al., J Biol Chem, 278(6):4135-4144 (2003)), and were evaluated in mice with intracranial tumors. The same NPs can be adapted for drug delivery for treatment of a wide variety of CNS diseases because MMP2 is also highly expressed in the microenvironment of many other common neurological disorders, such as ischemic stroke (Lakhan S E, et al., Frontiers in Neurology, 4:32 (2013)) and TBI36.

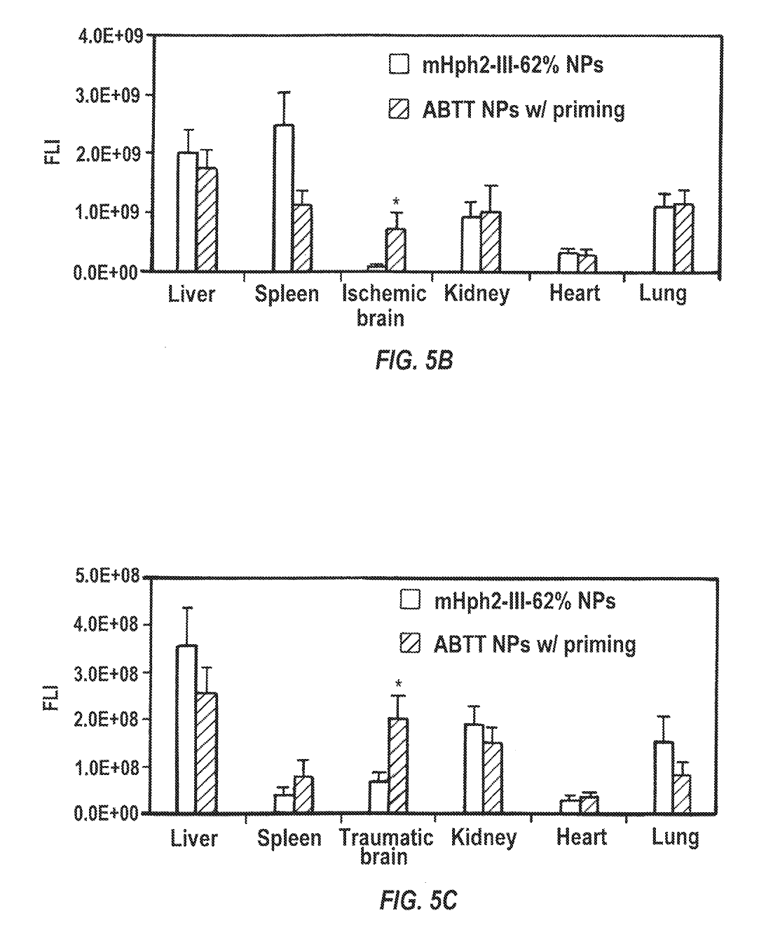

To demonstrate this versatility, the IR780-loaded ABTT NPs were evaluated in ischemic mice that underwent successful middle cerebral artery occlusion (MCAO) surgery, which was confirmed by cerebral blood flow and infarct measurements (FIG. 5A). IR780-loaded mHph2-III-62% NPs or IR780-loaded ABTT NPs were injected through the tail vein at a dose of 2 mg NPs/mouse/injection at 0, 24 and 48 hours after ischemia (1 hour) and reperfusion. At 72 hours, the organs were excised, and imaged using an IVIS imaging system. The results indicate that intravenous administration of ABTT NPs resulted in preferential accumulation of NPs in the ischemic region of the brain. The accumulation of ABTT NPs in the ischemic region was ˜8.9-fold higher than that of control mHph2-III-62% NPs (FIG. 5B). The signal of ABTT NPs in the brain was lower than that in the liver, likely due to the decreased blood flow in the ischemic region, which limited the circulation of NPs in the ischemic brain.

ABTT NPs were also evaluated in a controlled cortical impact-induced TBI mouse model, which, in contrast to the stroke model, has increased cerebral blood flow in the injury zone. Similar to the findings in the brain cancer and stroke models, the accumulation of NPs was significantly enhanced. The signal at the TBI region, which was comparable to that in liver, was 3.0-fold greater than that of control mHph2-III-62% NPs (FIG. 5C).

Systemic gene therapy for brain cancer is a major challenge, as successful delivery of a therapeutic dose requires penetration of both the BBB and cellular barriers with adequate efficiency. The above working Examples exemplify an innovative brain tumor-targeting drug delivery mechanism and tested it using a poly(amine-co-ester) terpolymer. The results show that NPs engineered through this mechanism efficiently overcame both barriers, resulting in an efficient approach for systemic delivery of gene therapy to brain cancer. In addition to their use for gene therapy, ABTT NPs may have broad applications for brain cancer management. For example, ABTT NPs can be engineered for brain cancer diagnosis, as our PET imaging and high-resolution confocal microscopy studies demonstrated that ABTT NPs were able to identify brain tumors including small satellite tumor islands containing a limited number of tumor cells. Such great sensitivity may be clinically useful in the diagnosis and treatment of small satellite tumor islands, which are not amenable to surgical resection and are often responsible for tumor relapse and death. ABTT NPs may also be adapted for brain cancer chemotherapy, as they demonstrated high capacity for loading hydrophobic agents, such as IR780 and LEXISCAN® used in this study. Furthermore, ABTT NPs may be repurposed for drug delivery for treatment of other CNS pathologies, such as stroke and TBI that were tested in this study. In summary, due to their unprecedented efficiency in crossing the BBB, their great capacity to accommodate and deliver cargo agents, and their construction from biodegradable materials with minimal toxicity, it is believed that that ABTT NPs can serve as a ground-breaking approach for the clinical management of a variety of neurological disorders.

### Example 8: ABTT NPs as a Vehicle for Systemic Treatment of Brain Cancer

### Materials and Methods

Nanoparticles were prepared similarly to those described above for Examples 1 and 6, but including the CTX targeting moiety as described in Example 6. Briefly, terpolymeric ABTT NPs were synthesized according to standard emulsion procedure (Strohbehn G, et al., Journal of Neuro- oncology, 121(3):441-449 (2015); Zhou J, et al., Proc Natl Acad Sci USA, 110(29):11751-11756 (2013); Zhou J, et al., Biomaterials, 33(2):583-591 (2012)). Briefly, for synthesis of paclitaxel-loaded NPs, 100 mg mIII-62% in 2 mL DCM containing 2.5 mg LEXISCAN® and 20 mg paclitaxel was added dropwise to 4 mL 2.5% PVA under vortex and sonicated to form a oil/water emulsion. The emulsion was poured into a beaker containing 0.3% PVA and stirred for 3 h to evaporate DCM. NPs were collected by centrifugation at 20000 rpm for 30 min. The precipitate was suspended in PBS and reacted with thiolated CTX (32ₖg) for 1 h for 1 h at room temperature. Nanoparticles were collected, suspended in H₂O and lyophilized for storage and characterization.

### Results

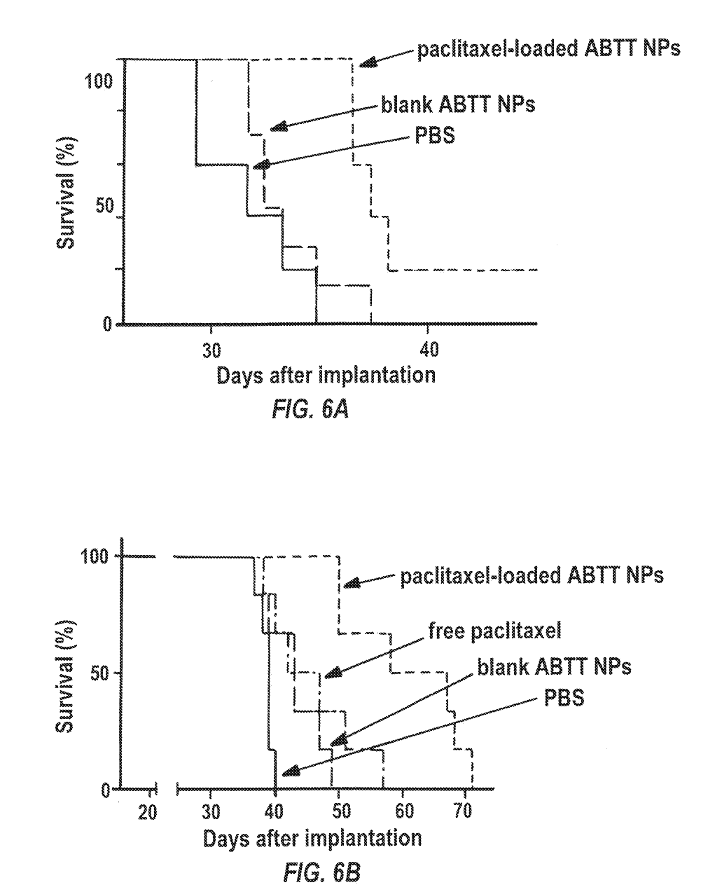

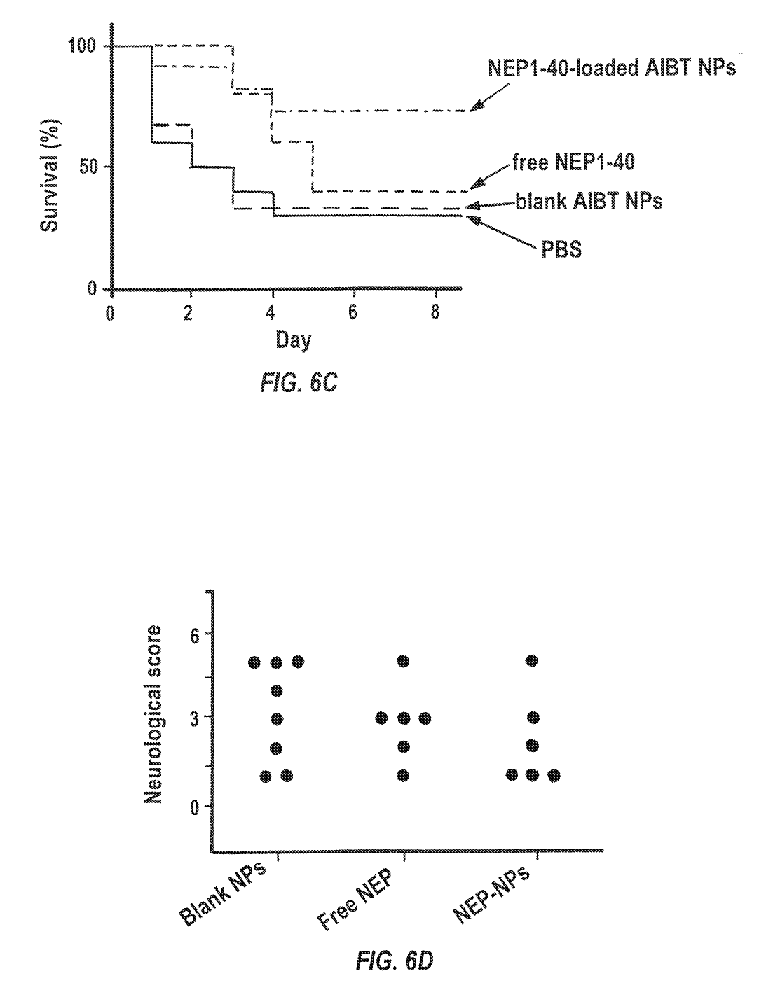

ABTT NPs were also evaluated for systemic delivery of chemotherapy to intracranial tumors. Paclitaxel, which was encapsulated into ABTT NPs in efficiency of 71%, was selected as a model drug. The paclitaxel-loaded ABTT NPs had spherical morphology and a diameter of ˜150 nm. First the paclitaxel-loaded ABTT NPs were evaluated in mice bearing GL261 gliomas. The intravenous treatment with the paclitaxel-loaded ABTT NPs significantly enhanced the median survival of tumor-bearing mice, which was 39 days, compared to 32 and 33 days for mice receiving saline and blank ABTT NPs, respectively (p<0.05) (FIG. 6A). The paclitaxel-loaded ABTT NPs were tested in a MDA-MB-231Br derived brain metastasis model, which forms multiple tumor lesions in the brain (Palmieri, et al., Cancer Research, 67(9): 4190-4198 (2007), Yoneda, et al., Journal Of Bone And Mineral Research: The Official Journal Of The American Society For Bone And Mineral Research, 16(8): 1486-1495 (2001)). As shown in FIG. 1B, the median survival time for mice receiving paclitaxel-loaded ABTT NPs of 63 days was significantly longer than for mice receiving PBS or free paclitaxel, which were 39 and 45, respectively (p<0.05).

The impact of treatments on volume and quantity tumor lesions in the brains of mice that were euthanized at day 35 following treatment with either blank or paclitaxel-loaded ABTT NPs was also examined. Consistent with the survival data, mice that received treatment with blank ABTT NPs had many large lesions in the brain, whereas the mice that received treatment of paclitaxel-loaded ABTT NPs had only one single small lesion. In both studies, mice were treated with nanoparticles for three times a week for three weeks at a dose of 2 mg/mouse. It is believed that enhanced efficacy can be achieved with a higher dose treatment (maximum tolerated dose >15 mg per treatment) or with extended treatment time. Taken together, this study indicated that systemic treatment of brain cancer is feasible and ABTT NPs represent a promising approach for this purpose.

### Example 9: PLGA Based ABTT NPs have Similar Brain-Tumor Targeting Effect

### Materials and Methods

PLGA based ABTT NPs were synthesized according to standard emulsion procedure (Strohbehn G, et al., Journal of Neuro-oncology, 121(3):441-449 (2015); Zhou J, et al., Proc Natl Acad Sci USA, 110(29):11751-11756 (2013); Zhou J, et al., Biomaterials, 33(2):583-591 (2012)). Briefly, for synthesis of IR780-loaded NPs, 100 mg PLGA in 2 mL DCM containing 2.5 mg LEXISCAN® and 1 mg IR780 was added dropwise to 4 mL 2.5% PVA under vortex and sonicated to form a oil/water emulsion. The emulsion was poured into a beaker containing 0.3% PVA and stirred for 3 h to evaporate DCM. NPs were collected by centrifugation at 20000 rpm for 30 min. The precipitate was suspended in PBS and reacted with thiolated CTX (32 μg) for 1 h at room temperature. Nanoparticles were collected, suspended in H₂O and lyophilized for storage and characterization.

### Results

ABTT NPs were synthesized using PLGA and found to have similar brain-tumor targeting effect in mice with GL261 gliomas as the counterpart CTX-mHph2-III-62% NPs loaded with LEXISCAN® discussed above.

### Example 10: Nanoparticles can Also be Used to Deliver Peptides to the Brain and are Also Effective for Use in Stroke Therapy

To address the challenge of systemic delivery of nanoparticles to the ischemic brain, a new strategy was developed and referred to as autocatalytic delivery of ischemic brain-targeted nanoparticles to the brain (FIG. 2A). Specifically, strategy includes synthesizing (autocatalytic ischemic brain targeted nanoparticles (AIBT NPs), a small fraction of which can enter the ischemic microenvironment through traditional mechanisms as described above. Immediately after reaching the ischemic region, nanoparticles will release BBB modulators, which in turn transiently enhance BBB permeability to allow additional nanoparticles to enter the same region. Through this mechanism, the delivery procedure creates a positive feedback loop. As a result, the efficiency of nanoparticle accumulation in the ischemic brain autocatalytically increases with time.

To test this mechanism, PLGA AIBT NPs were synthesized according to a recently published procedures (Zhou, et al., Biomaterials, 33(2): 583-591 (2012)). Briefly, ALGA ended with a carboxyl group was first activated and conjugated with poly(ϵ-carbobenzoxyl-L-lysine) (PLL). The resulting PLGA-PLL was subjected to standard emulsion procedures, after which resulting nanoparticles were collected and surface conjugated with polyethylene glycol (PEG) using a heterobifunctional PEG linker (NHS-PEG-Mal). In consistent with (Zhou, et al., Biomaterials, 33(2): 583-591 (2012)), the density of PEG on nanoparticle surface was as high as 9,600. The Mal end of PEG allows for versatile conjugation of thiolated ligands. To reach autocatalytic, ischemic brain-targeted delivery, chlorotoxin (CTX) was conjugated to the surface of nanoparticles and encapsulated LEXISCAN® internally. CTX is a 36-amino acid peptide with high specificity and affinity with matrix metalloproteinase 2 (MMP-2) (Deshane et al., J Biol Chem 2003, 278(6): 4135-4144 (2003)), which is up-regulated in the ischemic brain (Chang, et al., Journal Of Cerebral Blood Flow And Metabolism: Official Journal Of The International Society Of Cerebral Blood Flow And Metabolism, 23(12): 1408-1419 (2003) Heo, et al., Journal Of Cerebral Blood Flow And Metabolism: Official Journal Of The International Society Of Cerebral Blood Flow And Metabolism, (6): 624-633 (1999)). Lexiscan, a small molecule approved by the FDA for myocardial perfusion imaging, was recent shown to transiently enhance BBB permeability Carman et al., The Journal of neuroscience: the official journal of the Society for Neuroscience, 31(37): 13272-13280 (2011)). Resulting nanoparticles, called PLGA AIBT NPs, were of spherical morphology and in diameter of 121 nm. AIBT NP's major components and their functions are shown in Table 2.

TABLE 2
AIBT NP's major components and their functions

| Component | Function |
|---|---|
| PLGA | Provide protection of cargo |
| | agents and controlled drug |
| | release |
| PEG | 9,600 per nanoparticle, enhance |
| | circulation time |
| CTX | 774 per nanoparticle: provide |
| | targeted delivery through binding |
| | to MMP2 |
| Lexiscan | 1.0% (wt): provide autocatalysis |
| Cargo agent | Drug to be delivered |

PLGA AIBT NPs were evaluated for drug delivery to the ischemic mice, which were established through MCAO surgery with 1-hour occlusion. The success of surgery was validated by cerebral blood flow measurement. Only these showed reduction in cerebral blood flow by over 75% from baseline were included for this studies. Immediately after surgery, PLGA AIBT NPs loaded with IR780, a near-infrared fluorescence dye, were administered through tail vein injection. As controls, three separate groups of mice received treatment of the same amount of nanoparticles without modification or with modification of either CTX or LEXISCAN® alone (normalized based on IR780 signal). Three days later, mice were imaged using IVIS (Xenogen). AIBT NPs accumulated preferentially in the ischemic region in the brain in efficiency significantly greater than control nanoparticles, as demonstrated in both live animals and isolated brains. The IR780 signal was quantified in the brains and it was found that the concentration of AIBT NPs in the ischemic region was 8.2 fold greater than that of nanoparticles without modifications.

Next the use of AIBT NPs was evaluated as a nanocarrier for stroke treatment by using NEP1-40, a 40-aa peptide, as a model agent. Treatment with NEP1-40 peptide was recently reported to effectively reduce axonal injury and improve ischemia-induced neurologic outcomes in ischemic rats (Wang, et al., Neuroscience Letters, 417(3):6 (2007), Wang et al., Anesthesiology, 108(6):1071-1080 (2008), although the detailed molecular mechanism is yet to be investigated. First, procedures were developed to encapsulate NEP1-40 into ABIT NPs with efficiency of 33.8%. The loading of NEP1-40 in resulting nanoparticles was 1.1% by weight.

Next, the efficacy of intravenous administration of NEP1-40-loaded AIBT NPs was evaluated in ischemic mice. Mice received successful MCAO surgeries were randomly grouped and received administration of NEP1-40-loaded AIBT NPs, blank nanoparticles, free NEP1-40 or PBS, through tail vein injection immediately, 24 hours and 48 hours after removing the occluder. Mouse survival and behavior were monitored over 9 days. Two separate groups of mice were evaluated for survival and one group of mice was evaluated for behavior. Mouse behavior was assessed based on a neurological scoring system with minor modifications, with 1 representing no symptoms and 5 representing death (Wang, et al., Anesthesiology, 108(6):1071-1080 (2008)). Results shown in FIG. 6C-6D indicated that systemic treatment of NEP1-40-loaded AIBT NPs significantly improved ischemic mouse survival and behavior. In contrast, treatment of the same amount of free NEP1-40 showed limited therapeutic benefit. The impact of treatments on infarct volumes was also examined. Mice received the same surgical procedures and treatments as described above. Five days after MCAO, mice were euthanized and the brains were harvested, sliced and staining with TTC (2,3,5-triphenyltetrazolium chloride). It was found that intravenous administration of NEP1-40-loaded AIBT NPs reduced infarct volumes. These results, taken together, indicate AIBT NPs is a promising vehicle for delivery of therapeutics for stroke treatment.

1. A nanocarrier or conjugate of a therapeutic, prophylactic or diagnostic active agent comprising a targeting moiety and blood-brain barrier (BBB) modulator encapsulated, dispersed therein or conjugated thereto.

2. The nanocarrier of claim 1 comprising a therapeutic, prophylactic or diagnostic agent conjugated, encapsulated or dispersed therein.

3. The nanocarrier of claim 1, wherein the nanocarrier is selected from the group consisting of nanolipogels, polymeric particles, solid lipid particles, inorganic particles, liposomes, and multilamellar vesicles.

4. The nanocarrier of claim 3, wherein the nanocarriers have an average diameter of less than 1000 nm.

5. The nanocarrier of claim 3, wherein the polymeric particles comprise a polymer of Formula I.

6. The conjugate of claim 1 having targeting moiety and BBB modulator conjugated thereof by one or more linkers, wherein the one or more linkers may be cleavable.

7. The conjugate of claim 1 encapsulated, dispersed in or conjugated to a nanocarrier selected from the group consisting of nanolipogels, polymeric particles, solid lipid particles, inorganic particles, liposomes, and multilamellar vesicles.

8. The nanocarrier or conjugate of claim 1, wherein the targeting moiety preferentially or selectively targets the BBB, brain cells, or other brain tissue.

9. The nanocarrier or conjugate of claim 8, wherein the targeting moiety is selected from the group consisting of mHph2, the peptide chlorotoxin (CTX), transferrin, transferrin receptor binding antibody, lactoferrin, melanotransferrin, folic acid, and α-mannose.

10. The nanocarrier or conjugate of claim 1, wherein the targeting moiety targets cancer cells, a tumor microenvironment, or a combination thereof.

11. The nanocarrier or conjugate of claim 10, wherein the targeting moiety selectively or preferentially binds to a cancer antigen or a tumor antigen.

12. The nanocarrier or conjugate of claim 11, wherein the antigen is a brain cancer antigen or an antigen of supporting cells.

13. The nanocarrier or conjugate of claim 1, wherein the BBB modulator is an adenosine receptor (AR) signaling agonist.

14. The nanocarrier or conjugate of claim 1, wherein the BBB modulator is selected from the group consisting of NECA, regadenoson, minoxidil sulfate, borneol, and ST013006.

15. The nanocarrier or conjugate of claim 1, wherein the active agent is selected from the group consisting of a nucleic acid, peptide, lipid, glycolipid, glycoprotein, and small molecules.

16. The nanocarrier or conjugate of claim 15, wherein the nucleic acid encodes a protein.

17. The nanocarrier or conjugate of claim 15, wherein the active agent is selected from the group consisting of antisense molecules, aptamers, ribozymes, triplex forming oligonucleotides, external guide sequences, RNAi, CRISPR/Cas, zinc finger nucleases, and transcription activator-like effector nucleases (TALEN).

18. A pharmaceutical composition comprising the nanocarrier or conjugate of claim 1.

19. A method of treating a subject with brain cancer comprising administering to the subject the nanocarrier or conjugate of claim 1 in an effective amount to increase the permeability of the blood brain barrier.

20. The method of claim 19, wherein the active agent is a chemotherapeutic agent and the nanocarrier or conjugate is administered to the subject in an effective amount to prevent or alleviate one or more symptoms of the cancer.

21. The method of claim 20, wherein the symptom is tumor burden.

22. The method of claim 19, wherein the cancer is selected from the group consisting of oligodendroglioma, meningioma, supratentorial ependymona, pineal region tumors, medulloblastoma, cerebellar astrocytoma, infratentorial ependymona, brainstem glioma, schwannomas, pituitary tumors, craniopharyngioma, optic glioma, and astrocytoma.

23. A method of treating a subject with neurological or neurodegenerative disease or disorder comprising administering the subject the nanocarrier or conjugate of claim 1 in an effective amount to increase the permeability of the blood brain barrier.

24. The method of claim 23, wherein the active agent is a neurological agent and the nanocarrier or conjugate is administered to the subject in an effective amount to prevent or alleviate one or more symptoms of the disease or disorder.

25. The method of claim 23, wherein the subject has, or is likely to develop, a condition selected from the group consisting of stroke, a traumatic brain injury, epilepsy, Huntington's Disease (HD), Amyotrophic Lateral Sclerosis (ALS), Parkinson's Disease (PD) and PD-related disorders, Alzheimer's Disease (AD) and other dementias, Prion Diseases such as Creutzfeldt-Jakob Disease, Corticobasal Degeneration, Frontotemporal Dementia, HIV-Related Cognitive Impairment, Mild Cognitive Impairment, Motor Neuron Diseases (MND), Spinocerebellar Ataxia (SCA), Spinal Muscular Atrophy (SMA), Friedreich's Ataxia, Lewy Body Disease, Alpers' Disease, Batten Disease, Cerebro-Oculo-Facio-Skeletal Syndrome, Corticobasal Degeneration, Gerstmann-Straussler-Scheinker Disease, Kuru, Leigh's Disease, Monomelic Amyotrophy, Multiple System Atrophy, Multiple System Atrophy With Orthostatic Hypotension (Shy-Drager Syndrome), Multiple Sclerosis (MS), Neurodegeneration with Brain Iron Accumulation, Opsoclonus Myoclonus, Posterior Cortical Atrophy, Primary Progressive Aphasia, Progressive Supranuclear Palsy, Vascular Dementia, Progressive Multifocal Leukoencephalopathy, Dementia with Lewy Bodies, Lacunar syndromes, Hydrocephalus, Wernicke-Korsakoff's syndrome, post-encephalitic dementia, cancer and chemotherapy-associated cognitive impairment and dementia, depression-induced dementia, pseudodementia, a spinal cord injury, post-traumatic stress syndrome, or a combination thereof.

26. A method of imaging a subject comprising administering the subject the nanocarrier or conjugate of claim 1, wherein the active agent is a diagnostic agent, and the blood-brain modulator is in an effective amount to increase the permeability of the blood brain barrier and acquiring at least one image of at least a portion of the subject.

27. The method of claim 26, wherein the imaging is carried out by magnetic resonance imaging (MRI), positron emission tomography (PET), single-photon emission computed tomography (SPECT) or optical imaging (OI).

28. The method of claim 27, wherein the diagnostic agent comprises a fluorophore or radioisotope.

29. The method of claim 28, wherein the radioisotope is selected from the group consisting of ¹¹C, ¹³N, ¹⁸F, ⁷⁶Br, ¹²³I, ¹²⁴I, ¹²⁵I, ¹³¹I, ⁹⁹mTC, ⁹⁵Tc, ¹¹¹In, ⁶²Cu, ⁶⁴Cu, ⁶⁷Ga, and ⁶⁸Ga.

30. A method of increasing the permeability of the blood-brain barrier comprising administering to a subject in need thereof a pharmaceutical composition comprising the nanocarriers or conjugates of claim 1.

31. The method of claim 30, wherein the active agent is in a separate nanocarrier, or is administered to the subject in free or soluble form in conjunction with the nanocarriers, or as the conjugate.

32. The method of claim 31, wherein the active agent is administered to the subject at a different time than the nanocarrier.

33. The method of claim 31, wherein the active agent is administered after the nanocarrier.

34. A method of treating stroke comprising administering to a subject in need thereof the nanocarrier or conjugate of claim 1 in an effective amount to increase the permeability of the blood brain barrier.

35. A method of treating epilepsy comprising administering to a subject in need thereof the nanocarrier or conjugate of claim 1 in an effective amount to increase the permeability of the blood brain barrier.

36. A method of treating traumatic brain injury comprising administering to a subject in need thereof the nanocarrier or conjugate of claim 1 in an effective amount to increase the permeability of the blood brain barrier.


SEQUENCE LISTING (1 sequence)

SEQ ID NO: 1 - protein, 22 aa, Artificial Sequence
  Synthetic Peptide
YARVRRRGPRRHHHHHHHHHHC